(19) United States
(12) Patent Application Publication
(10) Pub. No.: US 2026/0248922 A1
(43) Pub. Date: Aug. 27, 2026

(54) TREATMENT OF SIGNS, SYMPTOMS AND/OR COMPLICATIONS OF VIRAL, BACTERIAL, PROTOZOAL, AND/OR FUNGAL INFECTIONS BY HIGH PENETRATION PRODRUGS

(71) Applicant: Techfields Inc., Princeton, NJ (US)

(72) Inventors: Chongxi YU, Princeton, NJ (US); Lina XU, Suzhou, Jiangsu (CN)

(73) Assignee: Techfields Inc., Princeton, NJ (US)

(21) Appl. No.: 18/729,501

(22) PCT Filed: Jan. 12, 2023
(86) PCT No.: PCT/CN2023/071974

§ 371 Date: Jul. 16, 2024

(30) Foreign Application Priority Data
Jan. 17, 2022 (WO) PCT/CN2022/072269

(51) Int. Cl. A61K 47/54; A61P 11/00; A61P 29/00

(52) U.S. Cl. CPC A61K 47/545; A61K 47/542; A61P 11/00; A61P 29/00

(57) ABSTRACT

High penetration prodrugs (HPPs), pharmaceutical compositions and methods thereof, for treatment of signs, symptoms and/or complications of viral, bacterial, protozoal, and/or fungal infections are disclosed. The HPPs are capable of penetrating one or more biological barriers to reach areas that their corresponding parent drugs may not be able to reach with sufficient quantities. While the HPPs can be administered to a subject through various administration routes, e.g., local delivery or systemic administration, transdermal or topical administration of them provides various additional advantages over use of the parent drugs.

### FIELD OF THE DISCLOSURE

This application relates to the field of pharmaceutical compositions and methods for treating signs, symptoms and/or complications of viral, bacterial, protozoal, and/or fungal infections, in particular, using high penetration prodrugs for the treatment while preventing the immune system from overreactions and avoiding or minimizing other side effects.

### BACKGROUND OF THE DISCLOSURE

Bacteria can cause many types of infections in human body and other living beings, such as urinary tract infections, gastrointestinal system infections, ear infections, strep throat, and the like. Many bacteria may cause deadly diseases, such as tuberculosis, streptococcus, syphilis, staphylococcus, aspergillus, tetanus, Vibrio cholera, salmonella, Clostridium botulinum, E. Coli, etc. Antibiotics are used to treat bacterial infections; however, most bacteria reproduce by dividing every few hours, allowing them to evolve rapidly and adapt quickly to new environmental conditions. During replication, mutations arise and some of these mutations may help an individual bacterium survive exposure to an antibiotic. Infections caused by antibiotic-resistant germs are difficult, and sometimes impossible, to treat. Each year in the U.S., at least 2.8 million people are infected with antibiotic-resistant bacteria or fungi, and more than 35,000 people die as a result (CDC's Antibiotic Resistance Threats in the United States, 2019 (2019 AR Threats Report)).

Protozoa is an informal term for a group of single-celled eukaryotes, either free-living or parasitic, that feed on organic matter such as other microorganisms or organic tissues and debris. Protozoan infections are responsible for diseases that affect many types of organisms, including plants, animals, and some marine life. Many of the most prevalent and deadly human diseases are caused by a protozoan infection, including African sleeping sickness, amoebic dysentery, and malaria. The World Health Organization (WHO) estimates that in 2019 there were 229 million new cases of malaria resulting in 409,000 deaths (WHO, World Malaria Report 2020). Malaria is a mosquito-borne infectious disease that affects humans and other animals and causes symptoms that typically include fever, tiredness, vomiting, and headaches. In severe cases, it can cause yellow skin, seizures, coma, or death. Malaria has several severe complications, among these is the development of respiratory distress. Like bacteria, protozoa evolve rapidly and adapt quickly to new environmental conditions, thus becoming drug resistant. Drug resistance poses a growing problem in 21st-century malaria treatment (Sinha, S., et al., Parasite., 2014, 21:61). Resistance is now common against all classes of antimalarial drugs.

Fungi are multicelled, plant-like organisms. Many fungal infections, such as athlete's foot and yeast infections, are not dangerous in a healthy person. People with weak immune system (from diseases like HIV or cancer or serious virus infections), however, may get more serious fungal infections. Most fungi reproduce in very high speed, allowing them to evolve rapidly and adapt quickly to new environmental conditions, becoming drug resistant.

A virus is a submicroscopic infectious agent that replicates only inside the living cells of an organism. They need to use another cell's structures to reproduce. When they enter human body, viruses spread rapidly and can make a person sick. Examples of common human diseases caused by viruses include the common cold, influenza, chickenpox, and cold sores. Many serious diseases such as rabies, Ebola virus disease, AIDS (HIV), avian influenza, H1N1, SARS, and COVID-19 are caused by viruses. Viral infections in animals provoke an immune response that usually eliminates the infecting virus, however, human or animals' immune system may overreact and kill human or animals by their own immune system.

Viral, bacterial, protozoal, and fungal infections can be aggressive life-threatening infections. Common symptoms of most bacterial, protozoal, fungal, and viral infections include fatigue, loss of appetite, weight loss, fever, night sweats, chills, aches, inflammation, cough, shortness of breath, and pains. The classic symptoms of a bacterial infection are local redness, fever, swelling, and pain at the site of infection. Acute inflammations may cause pain, tissue redness (blood vessels and the capillaries leaking), tissue swelling, and damage the tissues, then help bacterial, protozoal, and/or fungal infection, especially, to infect cavities, urinary tract, gastrointestinal system, ear, eyes, and nose. In general, viral infections are systemic, involving many different parts of the body at the same time, causing, e.g., runny nose, cough, and body aches. In all cases, bacteria, fungi, and viruses infect one cell or one tissue and then start to spread around the body infecting multiple other cells or tissues, in some cases killing humans. All infections of viruses, becteria, protozoa, and/or fungi involve immune system reaction, causing inflammation and fever. Overactive or un-controllable immune system may cause un-controllabel high fever and severe inflammatory conditions, such as severe inflammations of lung, kidney, liver, brain and other tissues, and acute respiratory distress syndrome (ARDS). In particular, ARDS is a type of respiratory failure characterized by rapid onset of widespread inflammation in the lungs that causes low blood oxygen and can be life threatening. Symptoms include shortness of breath, rapid breathing, and bluish skin coloration. The inflammation may cause tissue fibroids and scarring and block immune cells enter the infection to kill viruses and cause lifelong or chronic infections, where the viruses continue to replicate in the body despite the host's defense mechanisms.

Current non-steroidal anti-inflammatory drugs (NSAIDs) do not significantly reduce total symptoms or duration of respiratory infections. Moreover, the use of NSAIDs is accompanied by an increased risk of significant gastrointestinal (GI) toxicity, including gastroduodenal bleeding, gastric ulcerations, gastritis, and GI perforation (see, e.g., Cohn S M, et al., J. Clin. Invest. 1997; 99 (6): 1367-1379; Tarnawski A S and Ahluwalia A., Curr. Med. Chem. 2012; 19 (1): 16-27; Fries J F, J. Rheumatol Suppl. 1991; 28:6-10; Garcia Rodriguez L A, et al., Arch. Intern Med. 1998; 158 (1): 33-9; and Richardson C, Emery P., Drug Saf. 1996; 15 (4): 249-60). NSAIDs have been associated with a clinically relevant 50-100% increase in the risk of myocardial infarction or cardiovascular death compared with placebo from a meta-analyses (see, Coxib and traditional NSAID Trialists' (CNT) Collaboration. The Lancet, 2013; 382:769-79).

In addition, there is also an urgent need to develop effective therapies for the outbreak of new diseases. For example, the novel coronavirus 2019 (COVID-19, or SARS-COV-2) has affected more than 275 million people and killed more than five million worldwide. Infection with COVID-19 in humans is associated with a broad spectrum of clinical respiratory syndromes, ranging from mild upper airway symptoms, such as, fever, headache, loss of taste or smell, sore throat, nausea, vomiting, diarrhea, cough, fatigue, anorexia, myalgias, and diarrhea, to progressive life-threatening viral pneumonia (Zhu, N., et al., N. Engl. J. Med., 2020, 382:727-33 and Chen, N., et al., Lancet, 2020, 395:507-13). With the rapid mutations of COVID and emergence of new variants, the human battle against the pandemic remains fierce perhaps in a distant future.

SARS-COV-2, the aetiological agent of COVID-19, causes the current pandemic. It produces protean manifestations ranging from head to toe, wreaking seemingly indiscriminate havoc on multiple organ systems, in particular the lungs, heart, brain, kidney, and vasculature. The concept of COVID-19 as an endothelial disease provides a unifying pathophysiological picture of this raging infection, and also provides a framework for a rational treatment strategy at a time when we possess an indeed modest evidence base to guide our therapeutic attempts to confront this novel pandemic (Peter Libby and Thomas Lüscher, European Heart Journal (2020) 41, 3038-3044).

### SUMMARY OF THE DISCLOSURE

The present disclosure provides high penetration prodrugs (HPPs) that can be used to treat signs, symptoms and/or complications of viruses, bacteria, protozoa, and/or fungi infections, while avoiding or minimizing the various aforementioned drawbacks of the existing therapies, such as NSAIDs.

In one aspect, the present disclosure provides a high penetration prodrug (HPP) of Structure L-1:

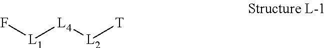

or a stereoisomer or pharmaceutically acceptable salt thereof, capable of penetrating one or more biological barriers for use in the treatment of a sign, symptom, and/or complication of a viral, bacterial, protozoal, and/or fungal infection, or a related condition thereof, wherein:
F, as a functional unit, is a molecular moiety of an active pharmaceutical ingredient (i.e., parent drug molecule);
T, as a transportational unit, is a basic group comprising a protonatable nitrogen;
L₁, L₂, and L₄ together form a linker such that the compound of Structure L-1 can be hydrolyzed or metabolized under physiological conditions to release the functional unit F to form the active pharmaceutical ingredient (i.e., the parent drug molecule), or a biologically active metabolite thereof.

In some embodiments, the functional unit (F) of the HPP is a moiety of a parent drug selected from 5-lipoxygenase-activating protein (FLAP) inhibitors, 5-lipoxygenase inhibitors, leukotriene receptor antagonists, aspirin and other nonsteroidal anti-inflammatory drugs (NSAIDs), other anti-inflammatory drugs, and any combinations thereof.

In some embodiments, the transportational unit (T) of an HPP of Structure L-1 comprises a protonatable amine group that is capable of facilitating the transportation or crossing of the HPP through one or more biological barriers; and the HPP is capable of being cleaved to release the active parent drug moiety, typically through hydrolysis under physiological conditions after being administered to a subject. In certain embodiments, the protonatable amine group is substantially protonated at a physiological pH. In certain embodiments, the amine group can be reversibly protonated. In certain embodiments, a transportational unit may be cleaved from the functional unit after the penetration of HPP through one or more biological barriers to reach a target site in a subject.

In some embodiments, the transportational unit of the HPP is a protonatable amine selected from a substituted primary amine, an unsubstituted primary amine, a substituted secondary amine, an unsubstituted secondary amine, a substituted tertiary amine, an unsubstituted tertiary amine, and a heterocyclyl group containing a protonatobale nitrogen in the ring.

In some embodiments, the linker (L₁, L₂, and La together) covalently links the functional unit and the transportational unit of the HPP, through a covalent bond that can be cleaved after the HPP penetrates across one or more biological barriers. The cleavable covalent bond can be a bond in a functional group selected from, for example, ester, amide, thioester, carbonate, carbamate, ether, thioether, phosphate, and oxime; in some preferred emobodiments, the functional group is an ester or amide.

In some embodiments, the linker (L₁, L₂, and L₄ together) is selected from C(═O)—O, C(═O)—S, C(═O)—NH, C(═O)—O—CH(L₃)-O, C(═O)—O—CH(L₃)-S, C(═O)—O—CH(L₃)-NH, C(═O)—S—CH(L₃)-O, C(═O)—S—CH(L₃)-S, C(═O)—S—CH(L₃)-NH, P(═O) (—O-L₃)—O, O—P(═O) (—O-L₃), C(═S)—O, C(═NH)—O, C(═NH)—S, C(═N-OL₃)-NH, C(═NH-OL₃)-O, C(═NH-OL₃)-S, O—C(═O), S—C(═O), NH—C(═O), O—CH(L₃)—O—C(═O), S—CH(L₃)-O—C(═O), O—CH(L₃)-S—C(═O), S—CH(L₃)-S—C(═O), O—CH(L₃)-NH—C(═O), O—C(═S), O—C(=N-OL₃), S—C(—N-OL₃), and NH—C(═N-OL₃), wherein L₃ at each occurrence is independently selected from H, substituted and unsubstituted alkyl, substituted and unsubstituted cycloalkyl, substituted and unsubstituted heterocyclyl, substituted and unsubstituted aryl, substituted and unsubstituted heteroaryl, substituted and unsubstituted alkoxy, substituted and unsubstituted alkylthio, and substituted and unsubstituted alkylamino.

Another aspect of the disclosure provides a pharmaceutical composition for treatment of signs, symptoms and/or complications of viral, bacterial, protozoal, and/or fungal infections and related conditions, comprising a high penetration prodrug (HPP) of the structure disclosed herein, and a pharmaceutically acceptable carrier.

The pharmaceutically acceptable carrier can be selected from alcohol, acetone, ester, cellulose, mannitol, croscarmellose sodium, vegetable oil, hydroxypropyl methylcellulose, water, and aqueous solution, and the like.

Another aspect of the disclosure provides a method of penetrating one or more biological barriers in a biological subject. The method comprises a step of administering to a biological subject an HPP or a pharmaceutical composition thereof. In certain embodiments, an HPP exhibits a penetration rate through one or more biological barriers about 20 times or higher, 50 times or higher, about 100 times or higher, about 200 time higher, about 300 times or higher, about 500 times or higher, or about 1,000 times or higher than its parent drug.

Another aspect of the disclosure provides a method of treating signs, symptoms and/or complications of a viral, bacterial, protozoal, and/or fungal infection, or a related condition, in a biological subject in need thereof by administrating to the subject a therapeutically effective amount of an HPP or a pharmaceutical composition thereof.

Another aspect of the disclosure provides use of an HPP of the disclosure in the manufacture of a medicament for treatment of signs, symptoms and complications of viral, bacterial, protozoal, and/or fungal infections, or related conditions, in a biological subject.

Another aspect of the disclosure provides a transdermal pharmaceutical composition comprising an HPP disclosed herein, which is adapted to be administered transdermally for treatment of signs, symptoms and/or complications of viral, bacterial, protozoal, and/or fungal infections and related conditions, the composition comprising a high penetration pro-drug of aspirin, another nonsteroidal anti-inflammatory drug (NSAID), another anti-inflammatory drug, or any combinations thereof.

Another aspect of the disclosure provides a method for treatment of signs, symptoms and/or complications of viral, bacterial, protozoal, and/or fungal infections and related conditions in a biological subject, comprising administrating to the biological subject a pharmaceutical composition comprising a high penetration prodrug as described above.

Other aspects and advantages of the disclosure will be better appreciated in view of the drawing, detailed description, examples, and claims below.

### BRIEF DESCRIPTION OF THE DRAWING

FIG. 1 illustrates the Kaplan-Meier Survival Curves of an efficacy study of the survival of Balb/c mice challenged with a lethal dose of mouse-adapted Influenza A PR/8/34 (H1N1) followed by treatment with HPPs.

### DETAILED DESCRIPTION OF THE DISCLOSURE

I. Treatment of Signs, Symptoms and/or Complications of Viral, Bacterial, Protozoal, and Fungal Infections

In one aspect, the disclosure provides HPPs for treatment of signs, symptoms and/or complications of viral, bacterial, protozoal, and/or fungal infections, such as fever, acute respiratory distress syndrome (ARDS), pneumonia, inflammations of brain, kidney, liver, blood vessels, and other tissues, and blood clotting (coagulation), or the like, caused by viruses, bacteria, protozoa, and/or fungi in humans and animals.

One aspect of the disclosure is directed to a strategy for the treatment of infections of viruses, bacteria, protozoa, and fungi, to relieve fever and inflammation and prevent the immune system overreaction to cause complications, but then let the immune system clean up viruses, bacteria, protozoa, and fungi in a well-controlled manner, so that all infections of viruses, bacteria, protozoa, and fungi will be very mild and well tolerated. The immune system can recognize and kill non-drug-resistant and drug-resistant viruses, bacteria, protozoa, and fungi equally so the problem of drug-resistance will be not a problem at all.

Prodrugs of the disclosure are designed to prevent the immune system from over-reaction to the viral, bacterial, protozoal, and fungal infections, however, pro-drugs of aspirin, diclofenac, and other anti-inflammatory drugs can kill viruses, bacteria, protozoa, and/or fungi.

In certain embodiments, the complication is pneumonia.

In certain embodiments, the complication is acute respiratory distress syndrome (ARDS).

In certain embodiments, the complication is chronic obstructive pulmonary disease (COPD).

In certain embodiments, the complication is blood clotting (coagulation).

In certain embodiments, the complication is heart attacks.

In certain embodiments, the complication is sepsis.

In certain embodiments, complications include acute respiratory distress syndrome, pneumonia, and inflammations of the brain, lung, kidney, liver, pancreas, GI system, blood vessels, and other tissues.

### II. Structures of High Penetration Prodrugs

One aspect of the disclosure provides a high penetration prodrug (HPP) that can be used to treat signs, symptoms and/or complications of viral, bacterial, protozoal, and/or fungal infection. The term “high penetration prodrug” or “HPP” as used herein refers to a compound comprising a functional unit covalently linked to a transportational unit through a linker, characterized by Structure L-1

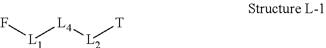

or a stereoisomer or pharmaceutically acceptable salt thereof, capable of penetrating one or more biological barriers for use in the treatment of a sign, symptom, and/or complication of a viral, bacterial, protozoal, and/or fungal infection, or a related condition thereof.

### Functional Unit (F)

The functional unit of an HPP comprises a moiety of a parent drug such that the HPP is capable of penetrating or crossing one or more biological barriers, and the HPP is capable of being cleaved so as to release the parent drug itself or a metabolite of the parent drug.

In certain embodiments, a functional unit may be hydrophilic, lipophilic, or amphiphilic (both hydrophilic and lipophilic). The lipophilic moiety of the functional unit may be inherent or achieved by converting one or more hydrophilic moieties of the functional unit to lipophilic moieties. For example, a lipophilic moiety of a functional unit is produced by converting one or more hydrophilic groups of the functional unit to lipophilic groups via organic synthesis. Examples of hydrophilic groups include, without limitation, carboxylic, hydroxyl, thiol, amine, phosphate/phosphonate, guanidine and carbonyl groups. Lipophilic moieties produced via the modification of these hydrophilic groups include, without limitation, ethers, thioethers, esters, thioesters, carbonates, carbamates, amides, phosphates and oximes. In certain embodiments, a functional unit is converted to a more lipophilic moiety through acetylation or acylation (alkanoylation). In certain embodiments, a functional unit is converted to a more lipophilic moiety via esterification.

In certain embodiments, a parent drug of an HPP is a drug that can be used by itself or in combination with other drug(s) to treat acute respiratory distress syndrome, pneumonia and other tissue inflammation caused by viruses and/or bacteria and/or fungi in humans and animals. Also, a related compound of a parent drug can be used, which is a compound comprising the structure of the parent drug, a metabolite of the parent drug, or an agent that can be metabolized into the parent drug or a metabolite of the parent drug after an HPP penetrates one or more biological barriers. A related compound of a parent drug further includes a compound that is an analog or mimic of the parent drug or a metabolite of the parent drug, or an agent that can be metabolized into an analog or mimic of the parent drug or a metabolite of the parent drug, after an HPP penetrates one or more biological barriers.

In certain embodiments, a functional unit is a molecular moiety of an active pharmaceutical ingredient (i.e., parent drug molecule) selected from 5-lipoxygenase-activating protein (FLAP) inhibitors, 5-lipoxygenase inhibitors, leukotriene receptor antagonists, and anti-inflammatory agents.

The moiety of a parent drug or the related compound thereof can be further converted to a lipophilic moiety as described supra. The main classes of drugs that can be used to treat acute respiratory distress syndrome, pneumonia, other tissue inflammation and other complications caused by viruses, bacteria, protozoa, and/or fungi in humans and animals include non-steroidal anti-inflammatory drugs (NSAIDs), leukotriene receptor antagonists, 5-lipoxygenase inhibitors, and 5-lipoxygenase-activating protein (FLAP) inhibitors.

### Transportational Unit (T)

The transportational unit of an HPP comprises a protonatable amine group that is capable of facilitating the transportation or crossing of the HPP through one or more biological barriers (e.g., >about 20 times, >about 50 times, >about 100 times, >about 300 times, >about 500 times, >about 1,000 times faster than the parent drug). In certain embodiments, the protonatable amine group is substantially protonated at a physiological pH. In certain embodiments, the amine group can be reversibly protonated. In certain embodiments, a transportational unit may be cleaved from the functional unit after the penetration of the HPP through one or more biological barriers.

In certain embodiments, the transportational unit is selected from Structure W-1, Structure W-2, Structure W-3, Structure W-4, Structure W-5, and Structure W-6:

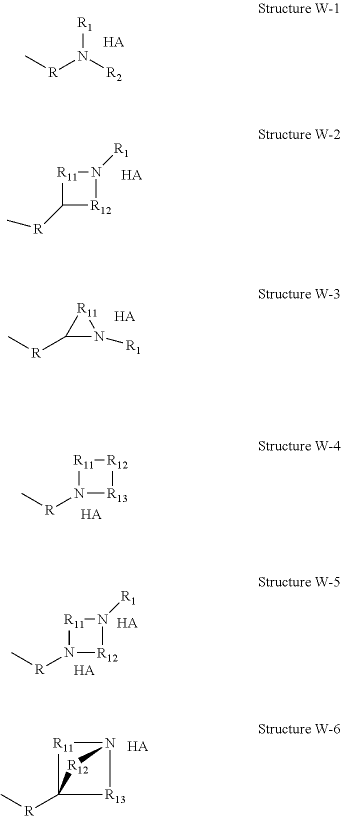

R at each occurrence is independently selected from a bond, substituted and unsubstituted alkylene, substituted and unsubstituted cycloalkylene, substituted and unsubstituted heterocyclylene, substituted and unsubstituted alkenylene, substituted and unsubstituted alkynylene, substituted and unsubstituted arylene, and substituted and unsubstituted heteroarylene, wherein any CH2 in R may be optionally further replaced with O, S, or NR₃, wherein R₃ is hydrogen, C₁-C₆ alkyl, C₃-C₆ cycloalkyl, or C₆-C₁₀ aryl;

R₁ and R₂ are independently selected from H, substituted and unsubstituted alkyl, substituted and unsubstituted cycloalkyl, substituted and unsubstituted heterocyclyl, substituted and unsubstituted alkyloxyl, substituted and unsubstituted alkenyl, substituted and unsubstituted alkynyl, substituted and unsubstituted aryl, and substituted and unsubstituted heteroaryl; or alternatively R₁ and R₂ together with the nitrogen atom to which they are attached form an optionally substituted heterocyclyl, which optionally further comprises one or two additional heteroatom(s) independently selected from O, S, and N;

R₁₁, R₁₂, and R₁₃ are each independently a bond, an optionally substituted C₁-C₄ alkylene, or an optionally substituted C₂-C₄ alkyenylene, wherein the alkylene or alkenylene optionally has one CH2 group replaced by O, S, or NR₃, wherein R₃ is hydrogen, C₁-C₆ alkyl, C₃-C₆ cycloalkyl, or C₆-C₁₀ aryl;
wherein any of the Ri in Structure W-2, Structure W-3 or Structure W-5 and the adjacent R₁₁ together with the nitrogen atom to which they are attached may form an optionally substituted heterocyclic ring, which may optionally further comprise one or two additional heteroatom(s) independently selected from O, S, and N; and
wherein the R₁₁ and R₁₂ or R₁₁ and R₁₃ in Structure W-2, Structure W-4, Structure W-5, or Structure W-6 may optionally be connected by an alkylene bridge, which is optionally substituted; and
wherein HA is selected from nothing and pharmaceutically acceptable acids, e.g., hydrochloride, hydrobromide, hydroiodide, nitric acid, sulfic acid, bisulfic acid, phosphoric acid, phosphorous acid, phosphonic acid, isonicotinic acid, acetic acid, lactic acid, salicylic acid, citric acid, tartaric acid, pantothenic acid, bitartaric acid, ascorbic acid, succinic acid, maleic acid, gentisinic acid, fumaric acid, gluconic acid, glucaronic acid, saccharic acid, formic acid, benzoic acid, glutamic acid, methanesulfonic acid, ethanesulfonic acid, benzensulfonic acid, p-toluenesulfonic acid and pamoic acid;
provided, however, that the structure forms a stable compound with no covalent bonding principles being violated.

In some embodiments, HA is nothing, and the HPP is a free base.

In some embodiments, R is a bond or C₁-C₆ alkylene.

In some embodiments, the transportational unit is Structure W-1, wherein R₁ and R₂ are each hydrogen or C₁-C₆ alkyl.

In some embodiments, the transportational unit is Structure W-2, Structure W-3, Structure W-4, Structure W-5, or Structure W-6, wherein R is a bond or C₁-C₄ alkylene; R₁ is hydrogen or C₁-C₆ alkyl; R₁₁ is a C₁-C₄ alkylene; R₁₂ and R₁₃ are independently a bond, CH₂, or CH₂CH₂.

In some embodiments, the transportational unit is a heterocyclyl selected from pyrrolidinyl, piperidinyl, piperazinyl, and morpholinyl, each of which is protonated with hydrochloride, hydrobromide, or acetic acid.

In some embodiments, R₁₁, R₁₂, and R₁₃ are each independently selected from CH₂, CH₂CH₂, CH—CH, CH₂CH₂CH₂, CH—CHCH2, CH₂CH₂CH₂CH₂, CH₂CH═CH—CH₂, CH₂CH₂CH₂CH₂CH₂, CH₂CH₂CH₂CH₂CH₂CH₂, each of which is optionally substituted.

The present disclosure encompasses any plausible combinations of the embodiments disclosed herein with regards to the structure of HPP.

### Linker (L)

The linker covalently linking a functional unit and a transportational unit of an HPP comprises a bond that is capable of being cleaved after the HPP penetrates across one or more biological barriers. The cleavable bond comprises, for example, a covalent bond, such as an ether, thioether, amide, ester, thioester, carbonate, carbamate, phosphate or oxime bond.

In some embodiments, L₁ is selected from a bond, O, S, —N(L₃)—, —N(L₃)-CH₂—O, —N(L₃)-CH₂—N(L₃)—, —O—CH₂—O—, —O—CH(L₃)-O, —S—CH(L₃)-O—; L₂ is selected from a bond, O, S, —N(L₃)—, —N(L₃)-CH₂—O, —N(L₃)-CH₂—N(L₃)—, —O—CH₂—O—, —O—CH(L₃)-O, —S—CH(L₃)—O—, —O-L₅-, —S-L₅-, and —N(L₃)-L₅-; and L₄ is selected from from a bond, C═O, C═S,

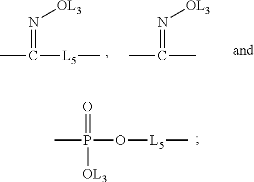

wherein L₃ at each occurrence is independently selected from H, substituted and unsubstituted alkyl, substituted and unsubstituted cycloalkyl, substituted and unsubstituted heterocyclyl, substituted and unsubstituted aryl, substituted and unsubstituted heteroaryl, substituted and unsubstituted alkoxy, substituted and unsubstituted alkylthio, and substituted and unsubstituted alkylamino; in certain embodiments, L₃ is independently H or C₁-C₆ alkyl;
L₅ at each occurrence is independently selected from a bond, substituted or unsubstituted alkylene, substituted or unsubstituted cycloalkylene, substituted and unsubstituted heterocycloalkylene, substituted and unsubstituted arylene, and substituted and unsubstituted heteroarylene; in certain embodiments, L₅ is independently selected from a bond, CH₂COOL₆, substituted or unsubstituted C₁-C₆ alkylene, substituted or unsubstituted C₃-C₆ cycloalkylene, substituted and unsubstituted 5- to 10-membered heterocycloalkylene, substituted and unsubstituted C₆-C₁₀ arylene, and substituted and unsubstituted 5- to 10-membered heteroarylene, wherein L₆ is a bond or substituted or unsubstituted C₁-C₆ alkylene.

In some embodiments, the linker (L₁, L₂, and L₄ together) is selected from C(═O)—O, C(═O)—S, C(═O)—NH, C(═O)—O—CH(L₃)-O, C(═O)—O—CH(L₃)-S, C(═O)—O—CH(L₃)-NH, C(═O)—S—CH(L₃)-O, C(═O)—S—CH(L₃)-S, C(═O)—S—CH(L₃)-NH, P(═O) (—O-L₃)-O, O—P(═O) (—O-L₃), C(═S)—O, C(═NH)—O, C(═NH)—S, C(═N-OL₃)-NH, C(═NH-OL₃)-O, C(═NH-OL₃)-S, O—C(═O), S—C(═O), NH—C(═O), O—CH(L₃)—O—C(═O), S—CH(L₃)-O—C(═O), O—CH(L₃)-S—C(═O), S—CH(L₃)-S—C(═O), O—CH(L₃)-NH—C(═O), O—C(═S), O—C(=N-OL₃), S—C(═N-OL₃), and NH—C(═N-OL₃), wherein L₃ at each occurrence is independently selected from H, substituted and unsubstituted alkyl, substituted and unsubstituted cycloalkyl, substituted and unsubstituted heterocyclyl, substituted and unsubstituted aryl, substituted and unsubstituted heteroaryl, substituted and unsubstituted alkoxy, substituted and unsubstituted alkylthio, and substituted and unsubstituted alkylamino; in certain embodiments, L₃ is independently H or C₁-C₆ alkyl.

In some embodiments, the HPPs are selected from Structure LRA-1, Structure LRA-2, Structure LRA-3, Structure LRA-4, Structure LRA-5, Structure LRA-6, Structure ARA-1, Structure ARA-2, Structure ARA-3, Structure ARA-4, Structure ARA-5, Structure ARA-6, Structure ARA-7, Structure ARA-8, Structure ARA-9, Structure ARA-11, Structure ARA-11, Structure ARA-12, Structure ARA-13, Structure ARA-14, Structure 5-LI-1, Structure 5-LI-2, Structure 5-LI-3, Structure 5-LI-4, Structure 5-LI-5, Structure 5-LI-6, Structure 5-LI-7, Structure 5-LI-8, Structure FLAP-1, Structure FLAP-2, Structure FLAP-3, Structure FLAP-4, Structure FLAP-5, Structure FLAP-6, Structure NSAID-1, Structure NSAID-2, Structure NSAID-3, Structure NSAID-4, Structure NSAID-5, Structure NSAID-6, Structure NSAID-7, Structure NSAID-8, Structure NSAID-9, Structure NSAID-10, Structure NSAID-11, Structure NSAID-12, and Structure NSAID-13:

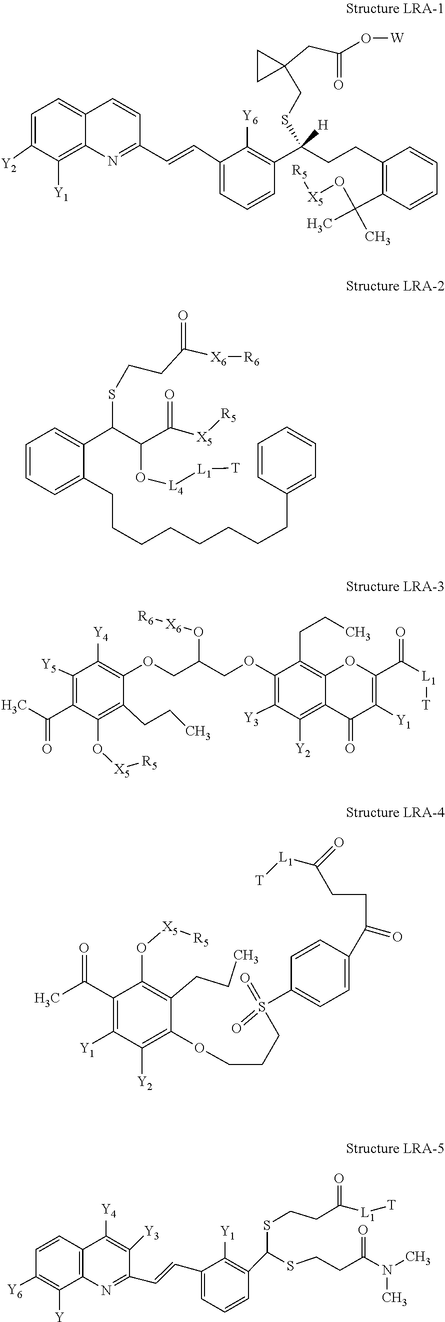

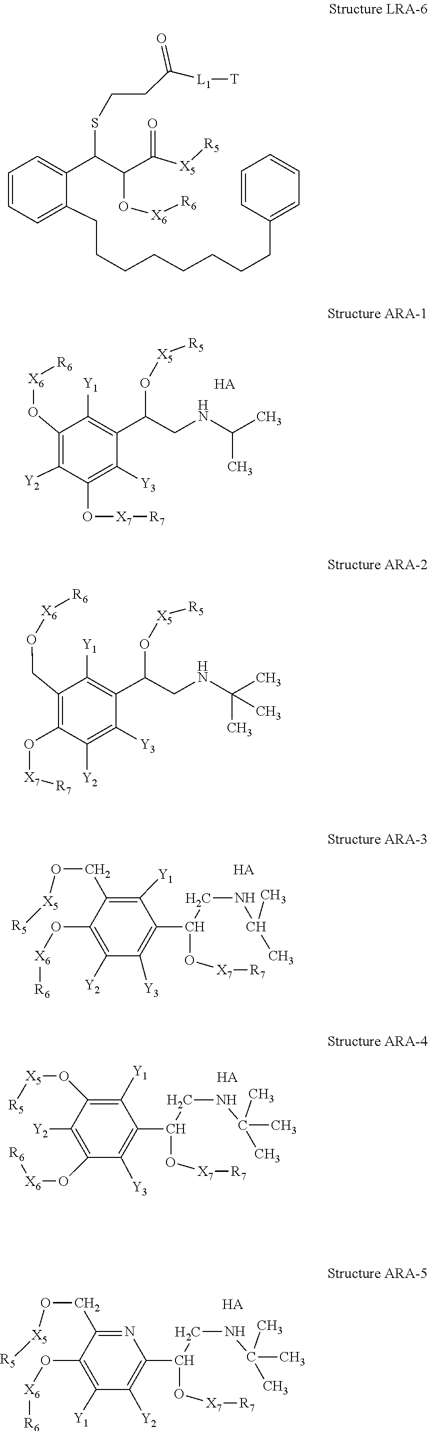

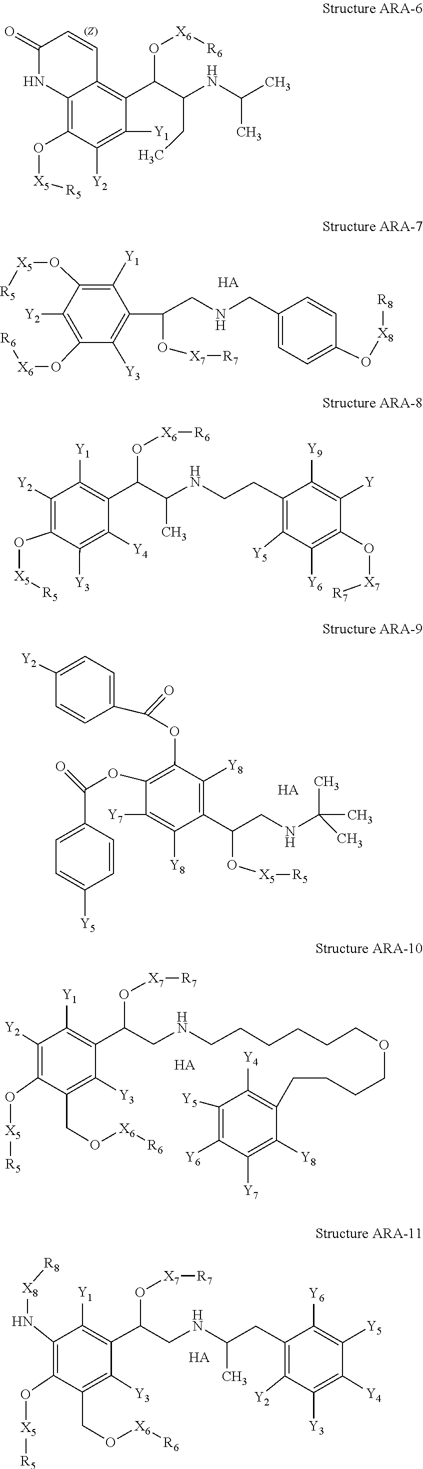

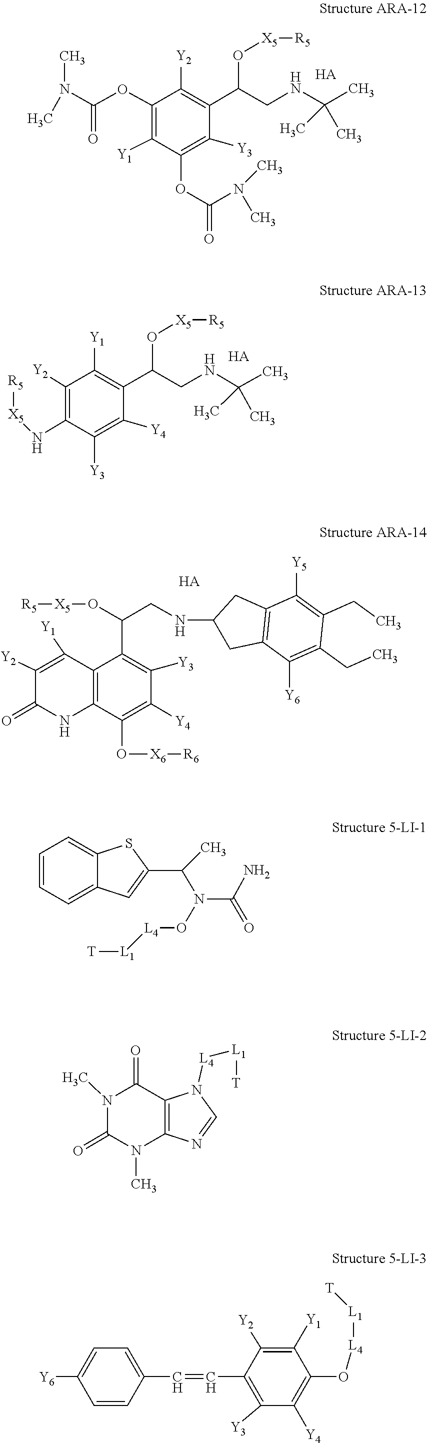

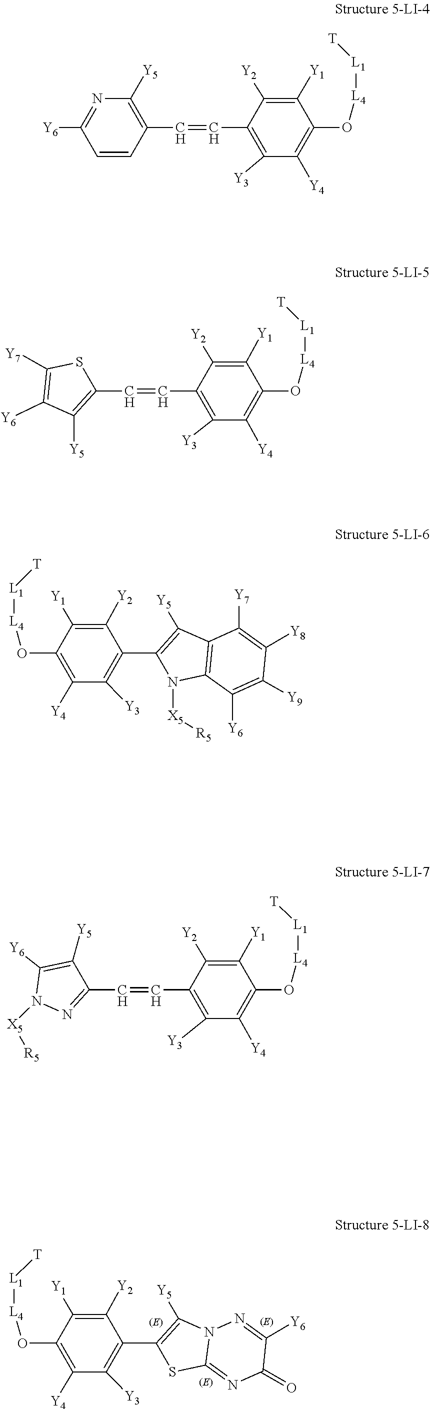

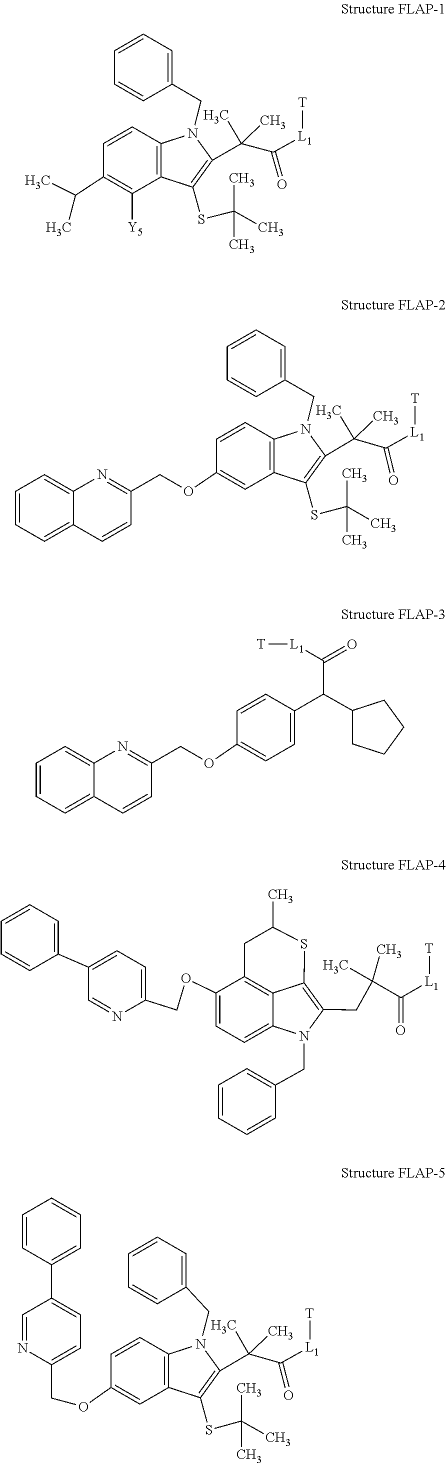

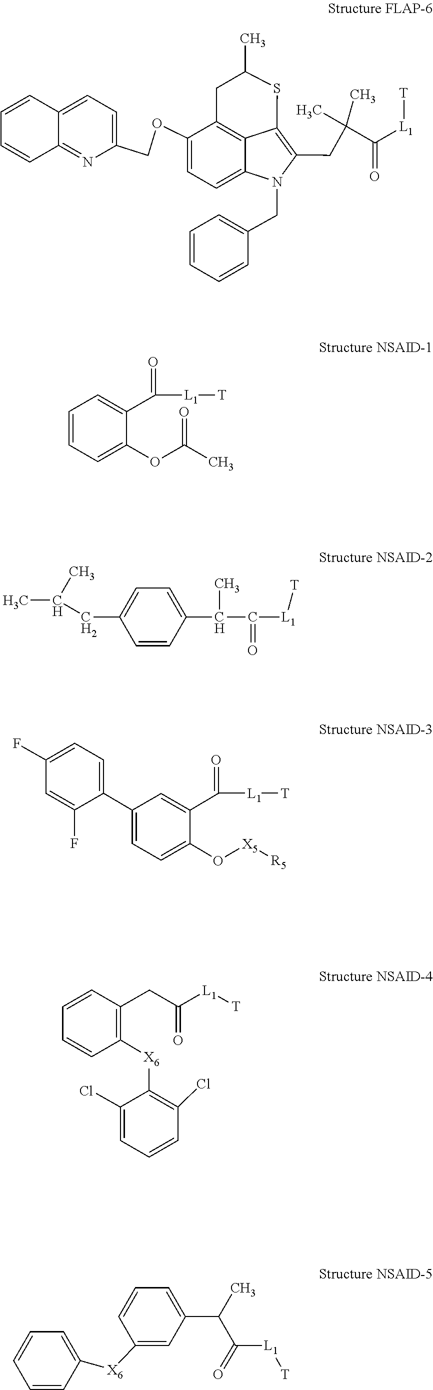

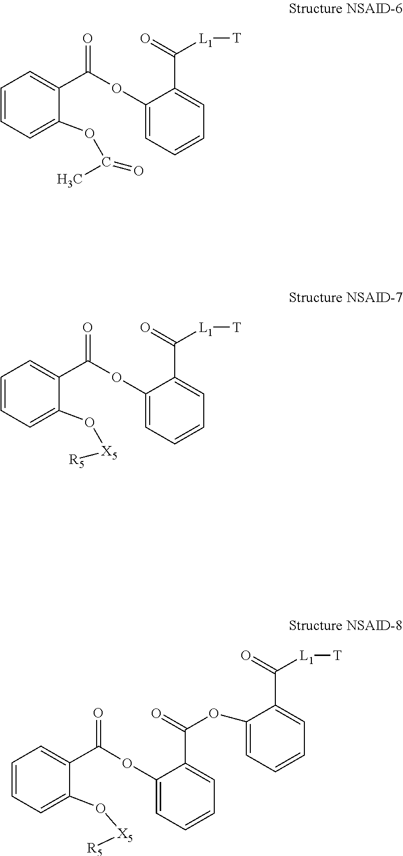

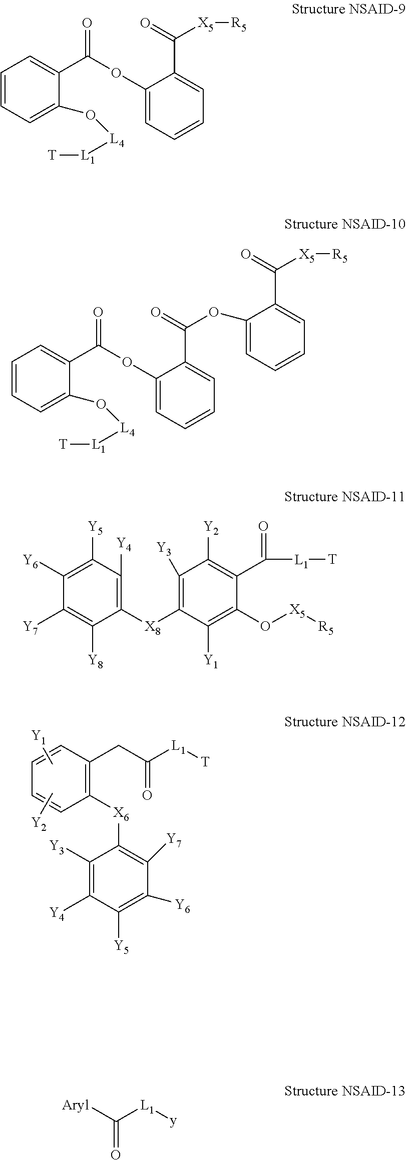

including stereoisomers and pharmaceutically acceptable salts thereof;
wherein Aryl- is a functional unit of an HPP of an anti-inflammatory drug or an anti-inflammatory drug-related compound, examples of Aryl-include, without limitation, Aryl-1, Aryl-2, Aryl-3, Aryl-4, Aryl-5, Aryl-6, Aryl-7, Aryl-8, Aryl-9, Aryl-10, Aryl-11, Aryl-12, Aryl-13, Aryl-14, Aryl-15, Aryl-16, Aryl-17, Aryl-18, Aryl-19, Aryl-20, Aryl-21, Aryl-22, Aryl-23, Aryl-24, Aryl-25, Aryl-26, Aryl-27, Aryl-28, Aryl-29, Aryl-30, Aryl-31, Aryl-32, Aryl-33, Aryl-34, Aryl-35, Aryl-36, Aryl-37, Aryl-38, Aryl-39, Aryl-40, Aryl-41, Aryl-42, Aryl-43, Aryl-44, Aryl-45, Aryl-46, Aryl-47, Aryl-48, Aryl-49, Aryl-50, Aryl-51, Aryl-52, Aryl-53, Aryl-54, Aryl-55, Aryl-56, Aryl-57, Aryl-58, Aryl-59, Aryl-60, Aryl-61, Aryl-62, Aryl-63, Aryl-64, Aryl-65, Aryl-66, Aryl-67, Aryl-68, Aryl-69, Aryl-70, and Aryl-71:

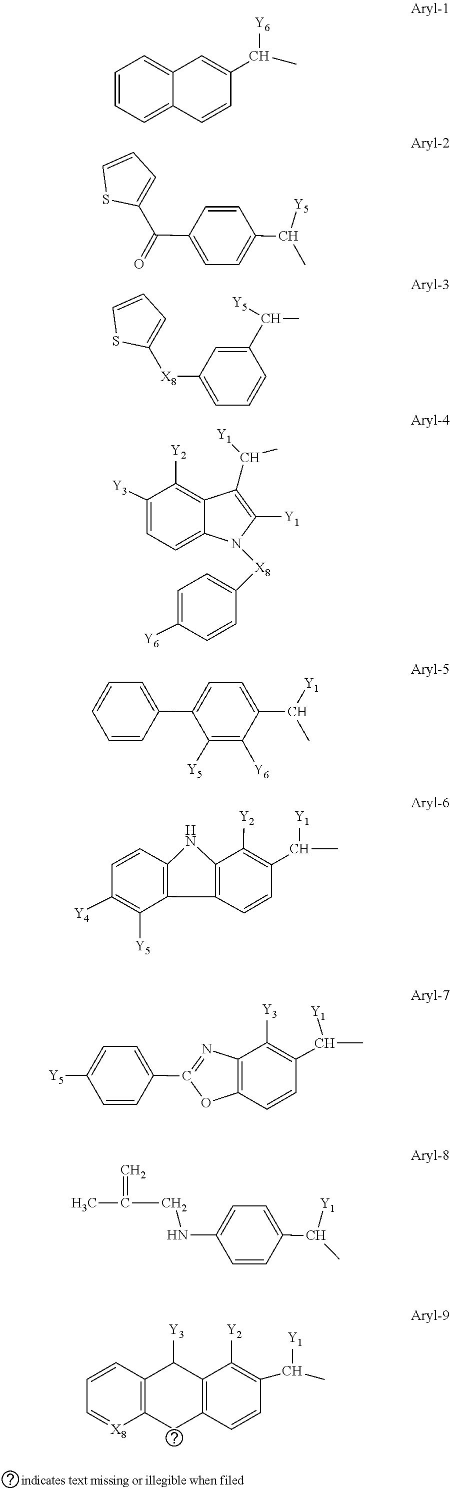

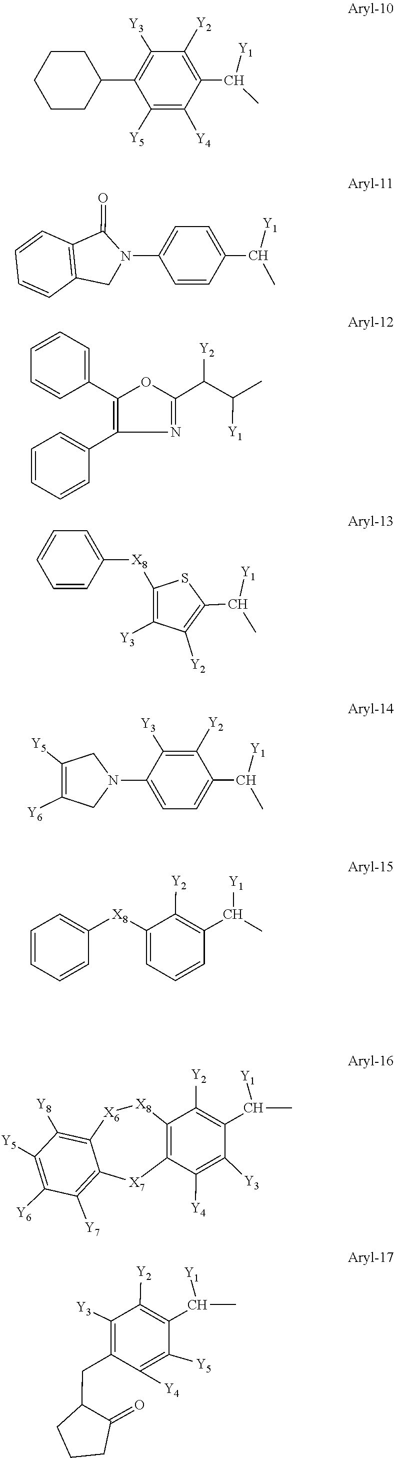

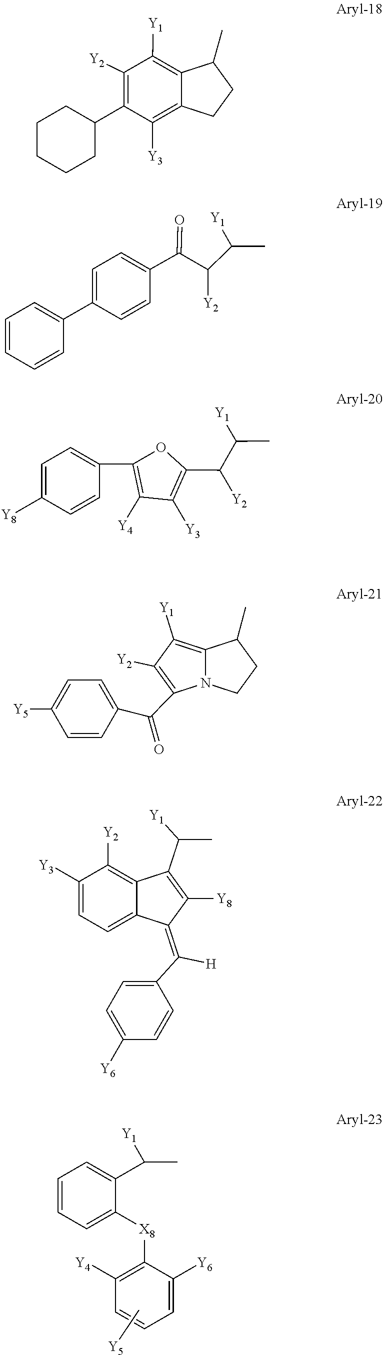

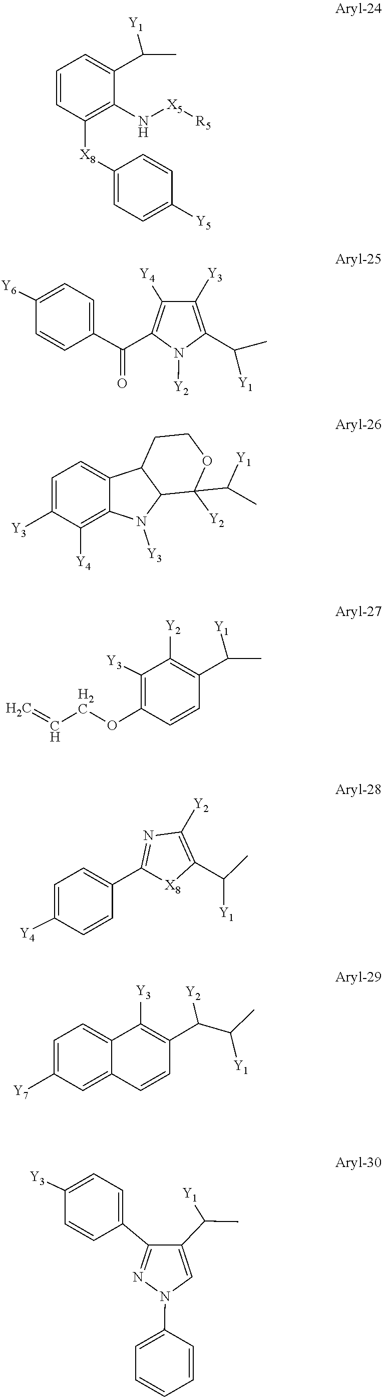

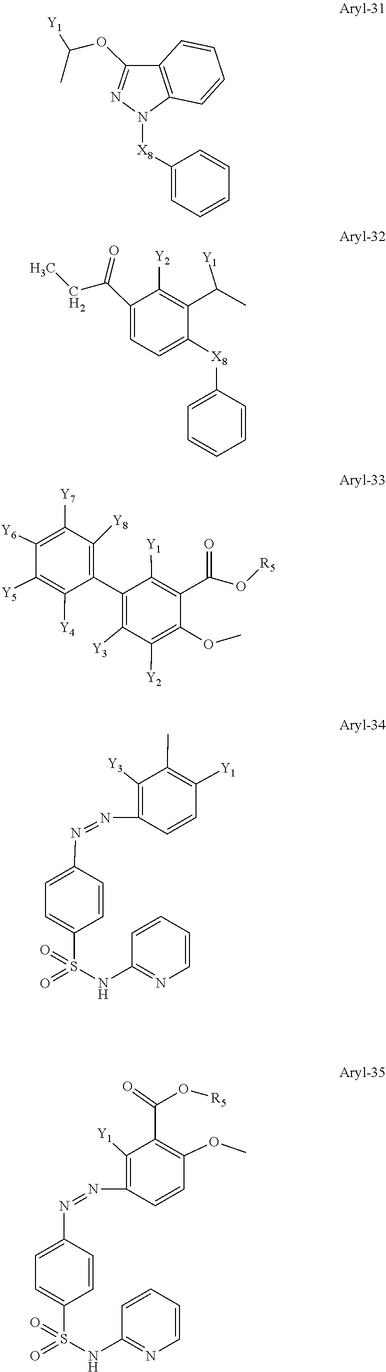

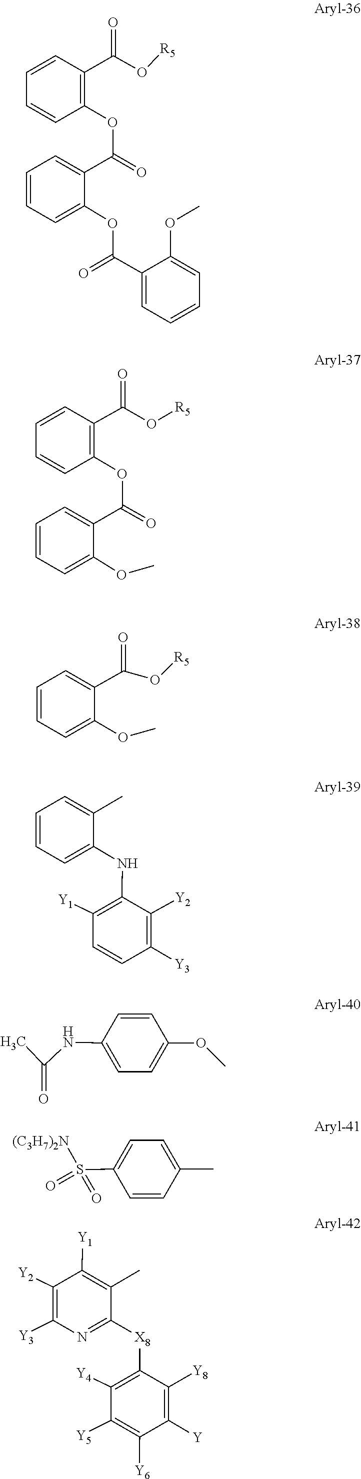

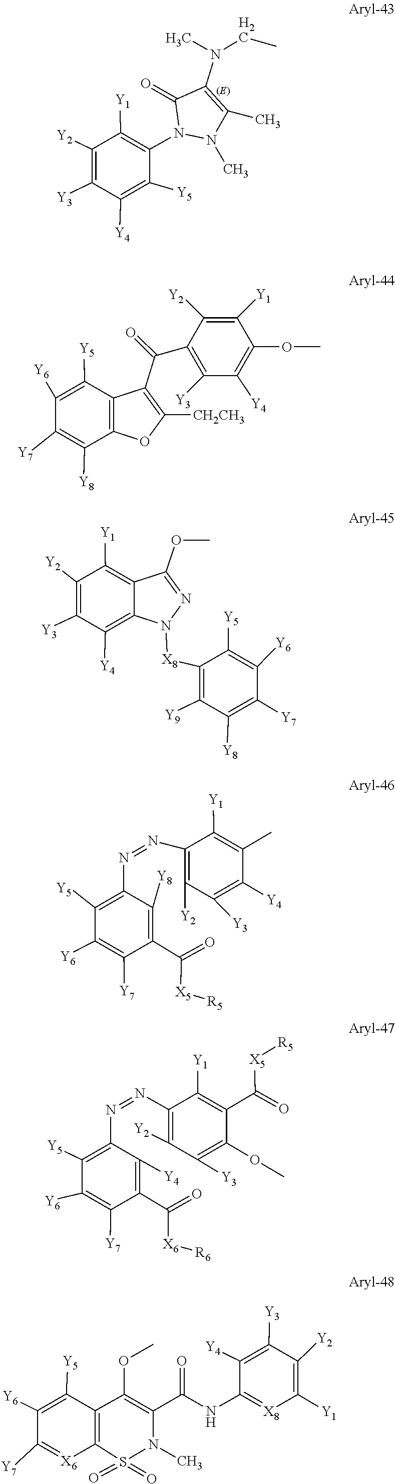

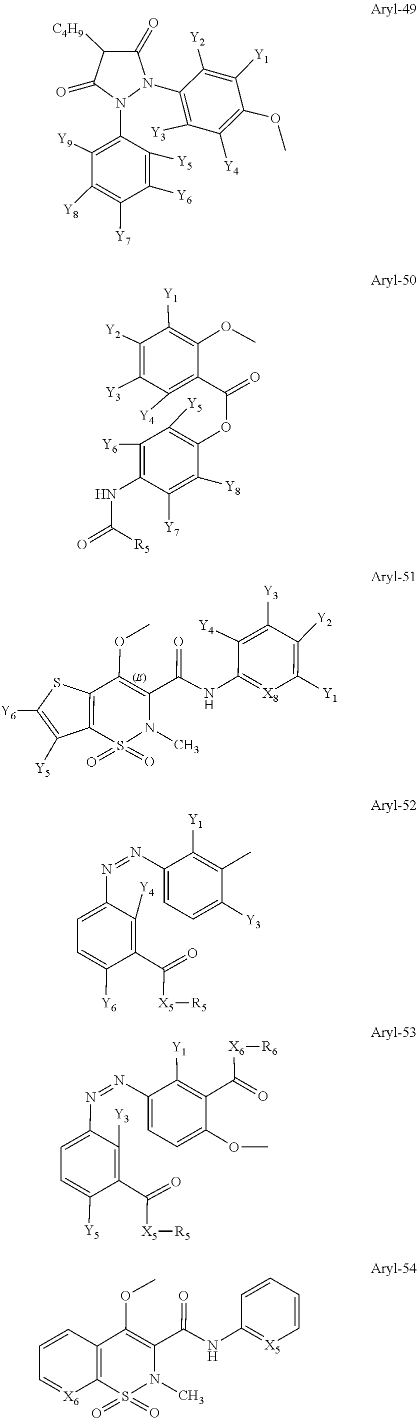

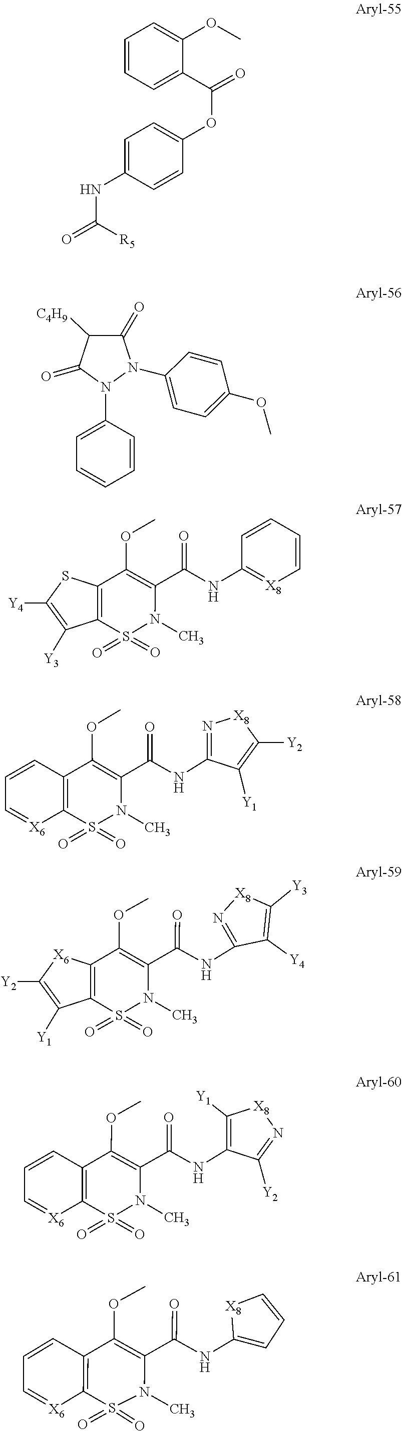

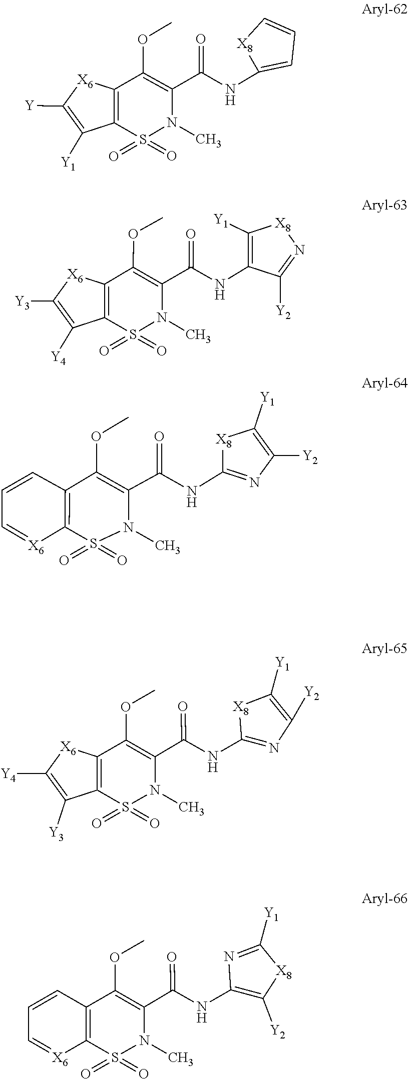

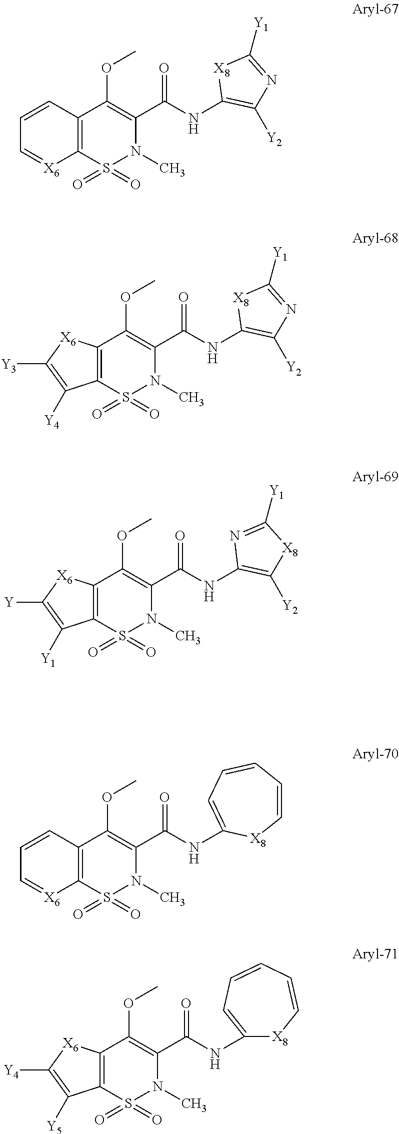

wherein:
   T is a transportational unit as defined above;
   L₁ is selected from nothing, O, S, —N(L₃)—, —N(L₃)-CH₂—O, —N(L₃)-CH₂—N(L₃)—, —O—CH₂—O—, —O—CH(L₃)-O, and —S—CH(L₃)-O—; and
   L₄ is selected from C—O, C═S,

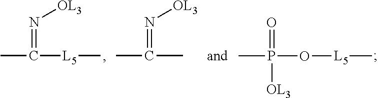

or
   L₁ and L₄ together is selected from C(═O)—O, C(═O)—S, C(═O)—NH, C(═O)—O—CH(L₃)-O, C(═O)—O—CH(L₃)-S, C(═O)—O—CH(L₃)-NH, C(═O)—S—CH(L₃)-O, C(═O)—S—CH(L₃)-S, C(═O)—S—CH(L₃)-NH, P(═O) (—O-L₃)-O, O—P(═O) (—O-L₃), C(═S)—O, C(═NH)—O, C(═NH)—S, C(═N-OL₃)-NH, C(═NH-OL₃)-O, C(═NH-OL₃)-S, O—C(═O), S—C(═O), NH—C(═O), O—CH(L₃)—O—C(═O), S—CH(L₃)—O—C(═O), O—CH(L₃)-S—C(═O), S—CH(L₃)-S—C(═O), O—CH(L₃)-NH—C(═O), O—C(═S), O—C(═N-OL₃), S—C(═N-OL₃), and NH—C(═N-OL₃);
   wherein L₃ at each occurrence is independently selected from H, substituted and unsubstituted alkyl, substituted and unsubstituted cycloalkyl, substituted and unsubstituted heterocyclyl, substituted and unsubstituted aryl, substituted and unsubstituted heteroaryl, substituted and unsubstituted alkoxy, substituted and unsubstituted alkylthio, and substituted and unsubstituted alkylamino; in certain embodiments, L₃ is independently H or C₁-C₆ alkyl;
   L₅ at each occurrence is independently selected from a bond, substituted or unsubstituted alkylene, substituted or unsubstituted cycloalkylene, substituted and unsubstituted heterocycloalkylene, substituted and unsubstituted arylene, and substituted and unsubstituted heteroarylene; in certain embodiments, L₅ is independently selected from a bond, CH₂COOL₆, substituted or unsubstituted C₁-C₆ alkylene, substituted or unsubstituted C₃-C₆ cycloalkylene, substituted and unsubstituted 5- to 10-membered heterocycloalkylene, substituted and unsubstituted C₆-C₁₀ arylene, and substituted and unsubstituted 5- to 10-membered heteroarylene, wherein L₆ is a bond or substituted or unsubstituted C₁-C₆ alkylene;
   X₅, X₆, X₇ and X₈ are independently selected from nothing, C(—O), C(═S), OC(═O), OC(═S), CH₂, CH, S, O and NR₅;
   Y, Y₁, Y₂, Y₃, Y₄, Y₅, Y₆, Y₇, and Y₈ are independently selected from H, OH, OW, OC(═O) W, L₁-L₄-L₂-W, OC(═O) CH₃, CH₃, C₂H5, C₃H₇, C₄H₉, R₆, SO₃R₆, CH₂OR₆, CH₂OC(═O) R₆, CH₂C(═O) OR₈, OCH₃, OC₂H₅, OR₆, CH₃SO₂, R₆SO₂, CH₃SO₃, R₆SO₃, NO₂, CN, CF₃, OCF₃, CH₂ (CH₂)ₙNR₅R₆, CH₂ (CH₂)ₙOR₆, CH(C(═O) NH₂) NHR₆, CH₂C(═O) NH₂, F, Br, I, C₁, CH═CHC(═O) NHCH₂C(═O) OW, CH—CHC(═O) NHCH₂. L₁-L₄-L₂-W, NR₈C(═O) R₅, SO₂NR₅R₈, C(═O) R₅, SR₅, R₆OOCCH (NHR₇) (CH₂)ₙC(═O) NH—, R₆OOCCH (NHR₇) (CH₂)ₙSC(═O) NH—, CF₃SCH₂C(═O) NH—, CF₃CH₂C(═O) NH—, CHF₂SCH₂C(═O) NH—, CH₂FSCH₂C(═O) NH—, NH₂C(═O) CHFS—CH₂C(═O) NH—, R₇NHCH (C(═O) OW) CH₂SCH₂C(═O) NH—, R₇NHCH (L₁-L₄-L₂-W) CH₂SCH₂C(═O) NH—, CNCH₂SCH₂C(═O) NH—, CH₃ (CH₂)ₙC(═O) NH—, R₇N═CHNR₇CH₂CH₂S—, R₇N=C(NHR₇) NHC(═O)—, R₇N=C(NHR₇) NHC(═O) CH₂, CH₃C (Cl)=CHCH₂SCH₂C(═O) NH—, (CH₃)₂C (OR₆)—, CNCH₂C(═O) NH—, CNCH₂CH₂S—, R₇HN═CH(NR₇) CH₂CH₂S—, CH₂—CHCH₂SCH₂C(═O) NH—, CH₃CH(OH)—, CH₃CH(OR₈)—, CH₃CH(Y₁)—, (CH₃)₂CH—, CH₃CH₂—, CH₃ (CH₂)ₙCH═CH(CH₂)ₘC(═O) NH—, substituted and unsubstituted alkoxyl, substituted and unsubstituted alkylthio, substituted and unsubstituted alkylamino, and substituted and unsubstituted alkylcarbonyl; R₅ is independently selected from H, C(═O) NH₂, CH₂CH₂OR₆, CH₂CH₂N (CH₃)₂, CH₂CH₂N (CH₂CH₃)₂, C₁, F, Br, I, substituted and unsubstituted alkyl, substituted and unsubstituted alkenyl, substituted and unsubstituted alkynyl, substituted and unsubstituted cycloalkyl, substituted and unsubstituted heterocycloalkyl, substituted and unsubstituted alkyloxyl, substituted and unsubstituted cycloalkyloxyl, substituted and unsubstituted aryl, substituted and unsubstituted heteroaryl, substituted and unsubstituted alkylcarbonyl, substituted and unsubstituted alkylamino, —C(—O)—W, L₁-L₄-L₂-W, and W;
R₆ is independently selected from H, F, Cl, Br, I, substituted and unsubstituted alkyl, substituted and unsubstituted cycloalkyl, substituted and unsubstituted heterocycloalkyl, substituted and unsubstituted alkenyl, substituted and unsubstituted alkynyl, substituted and unsubstituted alkyloxyl, substituted and unsubstituted cycloalkyloxyl, substituted and unsubstituted aryl, substituted and unsubstituted heteroaryl, —C(═O)—W,-L₁-L₄-L₂-W, and W;
R₇ being independently selected from H, F, Cl, Br, I, CH₃NHC(═O) CH₂CH(NHR₈) C(═O), R₅N=C(NHR₆) NHC(═O)—, C(═O) CH₃, C(═O) R₆, PO (OR₅) OR₆, substituted and unsubstituted alkyl, substituted and unsubstituted cycloalkyl, substituted and unsubstituted heterocycloalkyl, substituted and unsubstituted alkyloxyl, substituted and unsubstituted alkenyl, substituted and unsubstituted alkynyl, substituted and unsubstituted aryl, substituted and unsubstituted heteroaryl, substituted and unsubstituted alkylcarbonyl, substituted and unsubstituted alkylamino, L₁-L₄-L₂-W, and C—(═O)—W;
R₈ being independently selected from H, F, Cl, Br, I, CH₃, C₂H₅, CF₃, CH₂CH₂F, CH₂CH₂Cl, CH₂CH₂Br, CH₂CH₂I, CH₂CHF₂, CH₂CF₃, CH₂F, CH₂Cl, CH₂Br, CH₂I, CH₂NR₆R₇, CH(NHR₇) CH₂C(═O) NH₂, C₃H₇, C₄H₉, C₅H₁₁, R₆, C(═O) R₆, C(═O) NH₂, CH₂C(═O) NH₂, CH₂OC(═O) NH₂, PO (OR₅) OR₆, C(CH₃)₂C(═O) OR₆, CH(CH₃) C(═O) OR₆, CH₂C(═O) OR₆, C(═O)—W, and L₁-L₄-L₂-W;
L₂ at each occurrence is independently selected from a bond, O, S, —N(L₃)—, —N(L₃)-CH₂—O, —N(L₃)-CH₂—N(L₃)—, —O—CH₂—O—, —O—CH(L₃)-O, —S—CH(L₃)—O—, —O-L₅-, —S-L₅-, and —N(L₃)-L₅-; and
W is selected from H, substituted and unsubstituted alkyl, substituted and unsubstituted cycloalkyl, substituted and unsubstituted heterocycloalkyl, substituted and unsubstituted alkyloxy, substituted and unsubstituted alkenyl, substituted and unsubstituted alkynyl, substituted and unsubstituted aryl, substituted and unsubstituted heteroaryl, Structure W-1, Structure W-2, Structure W-3, Structure W-4, Structure W-5, and Structure W-6;
provided that the structure of HPP as defined forms a stable compound without any covalent bond principles being violated.

In some embodiments, the disclosure is directed to a transdermal pharmaceutical composition that is administered transdermally for treatment of signs, symptoms and/or complications of viral and/or bacterial, and/or protozoal, and/or fungal infections and related conditions, wherein the composition comprises an HPP molecule selected from 2-(diethylamino)ethyl 2-acetoxybenzoate hydrochloride, (pyrrolidin-2-yl)methyl 2-(2-(2,6-dichlorophenylamino)phenyl) acetate·hydrochloride, 2-(diethylamino)ethyl 2-(p-isobutylphenyl) propanoate·hydrochloride, 2-(N,N-diethylamino)ethyl 2-[1-[(1R)-1-[3-[2-(7-chloroquinolin-2-yl) ethenyl]phenyl]-3-[2-(2-hydroxypropan-2-yl)phenyl]propyl]sulfanylmethyl]cyclopropyl]acetate·hydrochloride, (RS)—N-[1-(1-benzothien-2-yl)ethyl]-N-(2-N,N-diethylaminoacetyloxy) urea·hydrochloride, (pyrrolidin-2-yl)methyl 2-cyclopentyl-2-[4-(quinolin-2-ylmethoxy)phenyl]acetate·hydrochloride, (pyrrolidin-2-yl)methyl 3-[[3-[(E)-2-(7-chloroquinolin-2-yl) ethenyl]phenyl]-[3-(dimethylamino)-3-oxopropyl]sulfanylmethyl]sulfanylpropanoate·hydrochloride, (Z)-2-(N,N-diethylaminoethyl) ethyl 2-(5-fluoro-2-methyl-1-(4-methylsulfinyl)benzylidene)-1H-inden-1-yl) acetate·HCl, 2-(dimethylamino)ethyl 2-(3-phenoxyphenyl) propionate·hydrochloride, S-(2-(dimethylamino)ethyl 2-(3-phenoxyphenyl) propanethioate hydrochloride, 2-(dipropylamino)ethyl 4-acetoxy-2′,4′-difluoro-[1,1′-biphenyl]-3-carboxylate hydrochloride, (pyrrolidin-2-yl)methyl 3-[1-(4-chlorobenzyl)-4-methyl-6-(5-phenylpyridin-2-yl) methoxy]-4,5-dihydro-1H-thiopyrano[2,3,4-c,d]indol-2-yl]-2,2-dimethylpropanoate hydrochloride. (pyrrolidin-2-yl)methyl 5-benzoyl-2,3-dihydro-1H-pyrrolizine-1-carboxylate hydrochloride, (pyrrolidin-2-yl)methyl 1,8-diethyl-1,3,4,9-tetrahydropyrano[3,4-b]indole-1-acetate hydrochloride, (pyrrolidin-2-yl)methyl 2-(2-Fluorobiphenyl-4-yl) propanoate hydrochloride, (pyrrolidin-2-yl)methyl 2-(6-methoxy-2~ naphthyl) propanoate hydrochloride. (pyrrolidin-2-yl)methyl 2-[(2,3~ dimethylphenyl)amino]benzoate hydrochloride, (pyrrolidin-2-yl)methyl 6-chloro-α-methylcarbazole-2-acetate hydrochloride, (pyrrolidin-2-yl)methyl 4-[4-[3-(4-acetyl-3-hydroxy-2-propylphenoxy) propylsulfonyl]phenyl]-4-oxo-butanoate hydrochloride, (pyrrolidin-2-yl)methyl 2-(2-(2,6-dichlorophenylamino)phenyl) acetate·hydrochloride, (pyrrolidin-2-yl)methyl (Z)-5-fluoro-2-methyl-1-[(4-methylsulfinyl)phenylmethylene]-1H-indene-3-acetate·HCl, (pyrrolidin-2-yl)methyl 1-methyl-5-(4-methylbenzoyl)-1H-pyrrole-2-acetate·HCl, (pyrrolidin-2-yl)methyl 1-(p-chlorobenzoyl)-5-methoxy-2-methylindole 3-acetate hydrochloride, (pyrrolidin-2-yl)methyl 5-(4-chlorobenzoyl)-1,4-dimethyl-1H-pyrrole-2-acetate hydrochloride, (pyrrolidin-2-yl)methyl 3-chloro-4-(2-propenyloxy)benzeneacetate hydrochloride, (pyrrolidin-2-yl)methyl 2-(p-isobutylphenyl) propanoate hydrochloride, (pyrrolidin-2-yl)methyl 2-acetoxybenzoate hydrochloride, (pyrrolidin-2-yl)methyl 2-(3-benzoylphenyl) propionate hydrochloride, (pyrrolidin-2-yl)methyl 2-(3-phenoxyphenyl) propionate hydrochloride, (pyrrolidin-2-yl)methyl (Z)-5-fluoro-2-methyl-1-[(4-methylsulfinyl)phenylmethylene]-1H-indene-3-ac etate hydrochloride, (pyrrolidin-2-yl)methyl 5-(4-chlorobenzoyl)-1,4-dimethyl-1H-pyrrole-2-acetate hydrochloride, (pyrrolidin-2-yl)methyl α-methyl-4-(2-thienylcarbonyl)benzeneacetate hydrochloride, (pyrrolidin-2-yl)methyl 6-chloro-α-methyl-9H-carbazole-2-acetate hydrochloride, (pyrrolidin-2-yl)methyl 2-(4-chlorophenyl)-α-methyl-5-benzoxazoleacetate hydrochloride, (pyrrolidin-2-yl)methyl 2-(5H-[1]-benzopyrano[2,3-b]pyridin-7-yl) propionate hydrochloride, (pyrrolidin-2-yl)methyl «-methyl-4-[(2-methyl-2-propen-1-yl)amino]benzeneacetate hydrochloride, (pyrrolidin-2-yl)methyl 2-[3-chloro-4-(3-pyrrolin-1-yl)phenyl]propanoate hydrochloride, (pyrrolidin-2-yl)methyl 10,11-dihydro-α-methyl-10-oxodibenzo[b,f]thiepin-2-acetate hydrochloride, (pyrrolidin-2-yl)methyl 2~ (8-methyl-5-oxo-6H-benzo[b][1]benzoxepin-3-yl)propanote hydrochloride, (pyrrolidin-2-yl)methyl 2-[4-[(2-oxocyclopentyl)methyl]phenyl]propanote hydrochloride, (pyrrolidin-2-yl)methyl 4-(1,3-dihydro-1-oxo-2H-isoindol-2-yl)-α-methylbenzeneacetate hydrochloride, (pyrrolidin-2-yl)methyl 2-chloro-2-(3-chloro-4-cyclohexylphenyl) acetate hydrochloride, (pyrrolidin-2-yl)methyl 4,5-diphenyl-2-oxazolepropanoate hydrochloride, (pyrrolidin-2-yl)methyl 3-(4-biphenylcarbonyl) propanoate hydrochloride, (pyrrolidin-2-yl)methyl 6-chloro-5-cyclohexyl-1-indancarboxylate hydrochloride, (pyrrolidin-2-yl)methyl 1-(p-chlorobenzoyl)-5-methoxy-2-methylindole 3-acetate hydrochloride, (pyrrolidin-2-yl)methyl 1-methyl-5-(4-methylbenzoyl)-1H-pyrrole-2-acetate hydrochloride, (pyrrolidin-2-yl)methyl 5-(4-chlorobenzoyl)-1,4-dimethyl-1H-pyrrole-2-acetate hydrochloride, (pyrrolidin-2-yl)methyl 3-chloro-4-(2-propenyloxy)benzeneacetate hydrochloride, (pyrrolidin-2-yl)methyl 3-2-(2,4-dichlorophenoxy)benzeneacetate hydrochloride, of (pyrrolidin-2-yl)methyl 4-acetamidophenyl salicylate hydrochloride. 2-1 (2,3~ dimethylphenyl)amino]benzoic acid, (pyrrolidin-2-yl)methyl 2-[[3-(trifluoromethyl)phenyl]amino]-3-pyridinecarboxylate hydrochloride, (pyrrolidin-2-yl)methyl 2-[(2,6-dichloro-3-methylphenyl)amino]benzoate hydrochloride, (pyrrolidin-2-yl)methyl salicylsalicylate hydrochloride, (pyrrolidin-2-yl)methyl 1-methyl-5-(4-methylbenzoyl)-1H-pyrrole-2-acetate hydrochloride, (pyrrolidine-2-yl)methyl 2-[[2-[(2,6-dichlorophenyl)amino]phenyl]acetoxy]acetate hydrochloride, or (pyrrolidine-2-yl)methyl 1-(p-chlorobenzoyl)-5-methoxy-2-methylindole 3-acetate hydrochloride.

In a method of treatment, the viral infections can be caused by one or more RNA viruses. In the method of treatment, the viral infections can be caused by one or more DNA viruses. In the method of treatment, the viral infections can be caused by one or more influenza viruses selected from influenza viruses A, B, C, and D. In the method of treatment, the viral infections can be caused by one or more Human influenza viruses selected from H1N1, H1N2 H1N7 H2N2, H2N3, H3N1, H3N2, H3N8, H5N1, H5N2, H5N3, H5N6, H5N8, H5N9, H6N1, H6N2, H6N5, H7N1, H7N2, H7N3, H7N4, H7N9, H7N7, H8N4, H9N2, H10N7, H10N8, H10N3, H11N2, H11N9, H12N5, H13N6, H17N10, and H18N11. In the method of treatment, the viral infections can be caused by one or more avian influenza A viruses selected from H1N1, H1N8, H2N9, H3N8, H3N2, H4N6, H4N3, H5N4, H5N8, H5N9, N5N1, H6N2, H6N1, H6N5, H6N8, H7N1, H8N4, H9N2, H9N2, H9N6, H9N7, H10N8, H11N6, H11N9, H12N5, H13N6, H14N4, and H15N9. In the method of treatment, the viral infections can be caused by one or more Swine influenza A viruses selected from H1N1, H1N2, H2N1, H3N2, and H2N3. In the method of treatment, the viral infections can be caused by one or more Equine influenza viruses selected from H3N8, and H7N7. In the method of treatment, the viral infections can be caused by one or more Canine influenza viruses selected from H3N2, H3N8, and H5N1. In the method of treatment, the viral infections can caused by one or more Cat influenza viruses selected from Feline herpes virus, Feline Calicivirus, Bordetella, bronchiseptica, and Chlamydophila felis. In the method of treatment, the viral infections can be caused by one or more coronaviruses. In the method of treatment, the viral infections can be caused by one or more of (SARS—Co-V) severe acute respiratory syndrome coronavirus, SARS-COV-1, SARS-COV-2 (COVID-19), 229E, NL63, OC₄₃, HKU1, MERS-COV, and the original SARS-COV.

In a method of treatment, the bacterial infections can be caused by one or more gram-positive bacteria. In the method of treatment, the bacterial infections can be caused by one or more gram-negative bacteria. In the method of treatment, the bacterial infections can be caused by one or more pathogenic bacteria. In the method of treatment, the pathogenic bacteria can be selected from tuberculosis, streptococcus, syphilis, staphylococcus, aspergillus, tetanus, Vibrio cholera, salmonella, Clostridium botulinum, and E. Coli.

In a method of treatment, the protozoal infections can be caused by one or more pathogenic protozoa that cause diseases in humans or animals. In the method of treatment, the pathogenic protozoa can be selected from Entamoeba histolytica (Amoebozoa), Acanthamoeba (Amoebozoa), Giardia lamblia (Metamonada), Trichomonas vaginalis (Metamonada), Dientamoeba fragilis (Metamonada), Trypanosoma brucei (Kinetoplastida), Trypanosoma cruzi (Kinetoplastida), Leishmania spp. (Kinetoplastida), Balantidium coli (Ciliate), Plasmodium spp. (Apicomplexa), Toxoplasma gondii (Apicomplexa), Babesia spp. (Apicomplexa), Cryptosporidium spp. (Apicomplexa), and Cyclospora cayetanensis (Apicomplexa).

In a method of treatment, the signs and symptoms of infection can include one or more of fatigue, loss of appetite, weight loss, fevers, night sweats, chills, aches, inflammation, cough, shortness of breath, pains, and runny nose.

In a method of treatment, the complications of infection can include one or more of pneumonia, acute respiratory distress syndrome, chronic obstructive pulmonary disease, blood clotting (coagulation), meningitis, encephalitis, cardiovascular disease, stroke, heart attack, sepsis, and inflammation of the brain, lung, kidney, liver, pancreas, GI system, blood vessels, and other tissues, such as fever, acute respiratory distress syndrome, pneumonia, inflammations of the brain, kidney, liver, blood vessels, and other tissues, blood clotting (coagulation) caused by viruses and/or bacteria and/or protozoa and/or fungi in human and animals.

As used herein, the term “pharmaceutically acceptable salt” means those salts of compounds of the invention that are safe for administration to a subject. Pharmaceutically acceptable salts include salts of acidic or basic groups present in compounds of the invention. Pharmaceutically acceptable acid addition salts include, but are not limited to, the hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,11-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts. For a review on pharmaceutically acceptable salts see BERGE ET AL., 66 J. PHARM. SCI. 1-19 (1977), incorporated herein by reference.

Unless defined otherwise, all technical and scientific terms used herein have the same meaning as commonly understood by one of ordinary skill in the art to which this invention belongs.

The term “alkyl” refers to a branched or unbranched monovalent aliphatic hydrocarbon radical derived from an alkane by removal of one hydrogen atom. In certain embodiments, an alkyl group contains 1 to 12 carbons. In certain embodiments, an alkyl group contains 1 to 8 carbons. In certain embodiments, sometimes preferably, an alkyl group contains 1 to 6 carbons, and in certain embodiments, sometimes more preferably, an alkyl group contains 1 to 4 carbons. Examples of alkyl include, but are not limited to, methyl, ethyl, propyl, isopropyl, butyl, isobutyl, t-butyl, pentyl, hexyl, heptyl, octyl, nonyl, decyl, undecyl, and dodecyl, or the like. The alkyl group can be substituted or unsubstituted.

The term “alkenyl” refers to any univalent aliphatic hydrocarbon radical derived from an alkene by removal of one hydrogen atom. In certain embodiments, an alkenyl group contains 2 to 12 carbons. In certain embodiments, an alkenyl group contains 2 to 8 carbons. In certain embodiments, sometimes preferably, an alkenyl group contains 2 to 6 carbons, and in certain embodiments, sometimes more preferably, an alkenyl group contains 2 to 4 carbons. Examples of alkenyl include, but are not limited to, ethenyl, propenyl, butenyl, isobutenyl, pentenyl, hexenyl, heptenyl, octenyl, nonenyl, decenyl, undecenyl, dodecenyl, or the like. The alkenyl group can be substituted or unsubstituted.

The term “alkynyl” refers to a univalent aliphatic hydrocarbon radical derived from an alkyne by removal of one hydrogen atom. In certain embodiments, an alkynyl group contains 2 to 12 carbons. In certain embodiments, an alkynyl group contains 2 to 8 carbons. In certain embodiments, sometimes preferably, an alkynyl group contains 2 to 6 carbons, and in certain embodiments, sometimes more preferably, an alkynyl group contains 2 to 4 carbons. Examples of alkynyl include, but are not limited to, ethynyl, propynyl, butynyl, isobutynyl, pentynyl, hexynyl, heptynyl, octynyl, nonynyl, decynyl, undecynyl, dodecynyl, or the like. The alkynyl group can be substituted or unsubstituted.

The term “cycloalkyl” refers to any univalent radical formed by removal of one hydrogen atom from a cycloalkane. In certain embodiments, cycloalkyl group contains 3 to 10 carbons. In certain embodiments, cycloalkyl group contains 3 to 8 carbons. In certain embodiments, sometimes preferably, cycloalkyl group contains 3 to 6 carbons. Examples of cycloalkyl include, but are not limited to, cyclopropyl, cyclobutyl, cyclopentyl, cyclohexyl, cycloheptyl, cyclooctyl, cyclononyl, cyclodecyl, cycloundecyl and cyclododecyl. The cycloalkyl is optionally substituted or unsubstituted.

The term “heterocyclyl” refers to a cycloalkyl wherein at least one ring atom is a non-carbon atom. Examples of the non-carbon ring atom include, but are not limited to, S, O and N. Representative examples of monocyclic heterocyclyls include, but are not limited to, pyrrolidyl, piperidyl, piperazinyl, morpholinyl, sulfo-morpholinyl, homopiperazinyl, and so on.

The term “alkylene” refers to a saturated linear or branched divalent aliphatic hydrocarbon group, derived by removing two hydrogen atoms from the parent alkane. The straight or branched chain group contains 1 to 12 carbon atom(s) (such as 1, 2, 3, 4, 5, 6, 7, 8, 9, 10, 11 and 12 carbon atom(s)), preferably 1 to 8 carbon atom(s), more preferably 1 to 6 carbon atom(s), and sometimes more preferably 1 to 4 carbon atom(s). Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylene (—CH₂CH₂CH₂CH₂—), etc. The alkylene group can be substituted or unsubstituted.

The term “alkenylene” refers to a divalent aliphatic hydrocarbon group that has at least two carbon atoms and at least one carbon-carbon double bond, preferably C₂-12 alkenylene, more preferably C₂-8 alkenylene, sometimes more preferably C₂-6 alkenylene, and sometimes even more preferably C₂-4 alkenylene. Non-limiting examples of alkenylene groups include, but are not limited to, —CH═CH—, —CH═CHCH₂—, —CH—CHCH₂CH₂—, —CH₂CH═CHCH₂-etc. The alkenylene group can be substituted or unsubstituted.

The term “aryl” refers to a 6 to 14 membered all-carbon monocyclic ring or a polycyclic fused ring (a “fused” ring system means that each ring in the system shares an adjacent pair of carbon atoms with another ring in the system) group, and has a completely conjugated pi-electron system. Preferably aryl is 6 to 10 membered, such as phenyl and naphthyl, most preferably phenyl. The aryl group can be substituted or unsubstituted.

The term “heteroaryl” refers to a 5 to 14 membered aromatic system having 1 to 4 heteroatom(s) selected from O, S and N as ring atoms. Preferably a heteroaryl is 5- to 10-membered (such as 5, 6, 7, 8, 9 and 10 membered), more preferably 5- or 6-membered, for example, thiadiazolyl, pyrazolyl, oxazolyl, oxadiazolyl, imidazolyl, triazolyl, thiazolyl, furyl, thienyl, pyridyl, pyrrolyl, N-alkyl pyrrolyl, pyrimidinyl, pyrazinyl, imidazolyl, tetrazolyl, and the like. The heteroaryl can be fused with the ring of an aryl, heterocyclyl or cycloalkyl, wherein the ring bound to parent structure is heteroaryl. The heteroaryl group can be substituted or unsubstituted.

The term “alkoxy” refers to —O-(alkyl), for example, methoxy, ethoxy, propoxy, butoxy, and the like.

The term “cycloalkoxy” refers to —O-(cycloalkyl), for example, cyclopropyloxy, cyclobutyloxy, cyclopentyloxy, cyclohexyloxy, and the like.

The term “bond” refers to a covalent bond using a sign of “—”.

The term “hydroxyl” refers to an —OH group.

The term “halogen” refers to fluoro, chloro, bromo or iodo atom.

The term “amino” refers to a —NH₂ group.

The term “alkylthio” refers to alkyl-S—.

The term “alkylamino” refers to “alkyl-NH—”, or sometimes dialkyl amino (—NR^{a}R^{b}), where the two alkyl groups (R^{a} and R^{b}) can be the same or different. Sometimes preferably, the alkyl group is a C₁-C₆ alkyl, and sometimes more preferably, the alkyl is a C₁-C₄ alkyl. Examples of alkylamino include, but are not limited to, CH₃—NH—, —N(CH₃)₂, —N(CH₂CH₃)₂, —NHCH₂CH₃, —N(CH₃) (CH₂CH₃), —NH-Bu^{t}, —N(CH₃)(Bu^{t}), or the like.

The term “cyano” refers to a —CN group.

The term “haloalkyl” means an alkyl group substituted by one or more halogen atoms, wherein the halogen atoms can be the same or different.

The term “nitro” refers to a —NO₂ group.

The term “oxo group” refers to a ═O group.

The term “carboxyl” refers to a —C(O) OH group.

The term “alkoxycarbonyl” refers to a —C(O) O (alkyl) group.

The term “alkylcarbonyl” refers to a —C(O)-alkyl group.

The term “optional” or “optionally” means that the event or circumstance described subsequently can, but need not, occur, and the description includes the instances in which the event or circumstance may or may not occur. For example, “the heterocyclyl group optionally substituted by an alkyl” means that an alkyl group can be, but need not be, present, and the description includes the case of the heterocyclyl group being substituted with an alkyl and the heterocyclyl group being not substituted with an alkyl.

The term “substituted” refers to one or more hydrogen atoms in the group, preferably up to 5, more preferably 1 to 3 hydrogen atom(s), independently substituted with a corresponding number of substituents. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

The term “covalent bonding principle”, as used herein, refers to those basic rules and principles in formation of covalent bonds in an organic compound, as generally understood by a person of ordinary skill in the art. For example, a carbon atom is tetravalent and can form only four covalent bonds (e.g., four single bonds, or a double bonds plus two single bonds, etc.), an oxygen is divalent and can only form two covalent bonds (two single bond in-O—, or a double bond in ═O).

The term “prodrug” refers to a compound that can be transformed in vivo to yield the active parent compound under physiological conditions, such as through hydrolysis in blood. Common examples include, but are not limited to, ester and amide forms of a compound having an active form bearing a carboxylic acid moiety. In particular, the present disclosure provides a unique class of prodrugs, namely “high penetration prodrugs”, as defined in the disclosure.

When any group in any HPP structure is indicated to be either “substituted” and/or “unsubstituted”, it means that the group can be optionally substituted by one or more, preferably one to five, and sometimes more preferably one to three, substituents independently selected from halogen, cyano, nitro, amino, alkyl, haloalkyl, alkoxy, haloalkoxy, aryl, alkylthio, alkylamino, alkylsulfonyl (alkylsulfone), alkylsulfoxyl (alkylsulfoxide), acyloxy, carboxylic acid, carboxylic ester, and carboxamide groups, or the like. The alkyl groups can be 1-10 carbon atoms, sometimes preferably 1-6 carbon atoms, sometimes more preferably 1-4 carbon atoms. The esters can be the esters of C₁ to C₁₀ alcohols, sometimes preferably C₁ to C₆ alcohols, sometimes more preferably C₁ to C₄ alcohols.

In some embodiments, when an alkyl, alkenyl, alkynyl, cycloalkyl, heterocyclyl, aryl, heteroaryl group, or the like, or a moiety thereof, is substituted, the substituent group(s) can be substituted at any available connection point(s), and the substituents can be one or more, sometimes preferably 1 to 5, and sometimes more preferably 1 to 3, group(s) independently selected from C₁-C₆ alkyl, halogen, C₁-C₆ alkoxy, C₁-C₆ alkenyl, C₁-C₆ alkynyl, C₁-C₆ alkylthio, C₁-C₆ alkylamino, di-(C₁-C₆ alkyl)amino, thiol, hydroxyl, nitro, cyano, amino, C₃-C₆ cycloalkyl, 5- to 10-membered heterocyclyl, C₆-C₁₀ aryl, 5- to 10-membered heteroaryl, C₃-C₆ cycloalkoxy, C₁-C₆ cycloalkylthio, 5- to 10-membered heterocyclylthio and oxo group. In some embodiments, sometimes preferably, the substituents are independently selected from C₁-C₆ alkyl, halogen, C₁-C₆ alkoxy, C₁-C₆ alkylthio, C₁-C₆ alkylamino, di-(C₁-C₆ alkyl)amino, thiol, hydroxyl, nitro, cyano, amino, and oxo group. In some embodiments, sometimes more preferably, the substituents are independently selected from C₁-C₄ alkyl, halogen, C₁-C₄ alkoxy, C₁-C₄ alkylthio, C₁-C₄ alkylamino, di-(C₁-C₄ alkyl)amino, thiol, hydroxyl, nitro, cyano, and amino. As a person of ordinary skill in the art would understand, an oxo (═O) group cannot be a substituent at an unsaturated carbon in any other group.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The terms “a” and “an” and “the” and similar references in the context of describing the invention (especially in the context of the following claims) are to be construed to cover both the singular and the plural, unless otherwise indicated herein or clearly contradicted by context. The use of the word “a” or “an” when used in conjunction with the term “comprising” in the claims and/or the specification may mean “one”, but it is also consistent with the meaning of “one or more”, “at least one”, and “one or more than one”.

Where the plural form is used for compounds, salts, and the like, this is taken to mean also a single compound, salt, or the like.

The term “and/or” as used herein refers to and encompasses any and all possible combinations of one or more of the associated listed items. When used in a list of two or more items, the term “and/or” means that any one of the listed items can be employed by itself, or any combination of two or more of the listed items can be employed. For example, if a composition, a combination, a constitution, a juxtaposition, or a group is described as including (or comprising) components A, B, C, and/or D, the composition can contain A alone; B alone; C alone; D alone; A and B in combination; A and C in combination; A and D in combination; B and C in combination; B and D in combination; C and D in combination; A, B, and C in combination; A, B, and D in combination; A, C, and D in combination; B, C, and D in combination; or A, B, C, and D in combination.

Throughout this application, the term “about” or “approximately” is used to indicate that a value includes the inherent variation of error for the device, the method being employed to determine the value, or the variation that exists among the study subjects. In one aspect, the terms “about” or “approximately” usually mean within 10%, in particular within 9%, in particular within 8%, in particular within 7%, in particular within 6%, in particular within 5%, in particular within 4%, in particular within 3%, in particular within 2%, in particular within 1%, in particular within 0.5% of a given value or range.

In some embodiments, the term “dose”, as used herein, means the amount of a drug or active component taken each time by an individual subject, in particular the total amount of a drug or active component taken each time by an individual subject, for one site.

In some embodiments, the term “dosage form”, as used herein, means a unit of administration of an active agent. Examples of dosage forms include tablets, capsules, injections, suspensions, liquids, emulsions, creams, ointments, suppositories, inhalable forms, transdermal forms, and the like.

In some embodiments, the term “unit dose” or “dosage unit” refers to a dosage form that is configured to deliver a specified quantity or dosage of composition or component thereof. Examples of dosage forms for topical administration include, but are not limited to, transdermal patch, cream, foam, gel, lotion, ointment, paste, powder, shake lotion, solid, sponge, tape, tincture, vapor, injection, drops, rinces, spray, and solution. A “unit dose” or “dosage unit” may be configured to provide a full unit dose or fraction thereof (e.g., ½, ⅓, or ¼ of a dose). A predetermined quantity in each unit dose can depend on factors that include, but are not limited to, the unique characteristics of the active compound and the particular therapeutic effect to be achieved, and the limitations inherent in the art of creating and administering such unit doses. For instance, a unit dose may be, a transdermal patch, a spray, i.e., once spray in the spray application, a droplet of the dropping application, a certain length of the tape, rice-sized or bean-sized ointment, or a scoop or a spoon of ointment. Unit dose measuring devices, such as a cup, scoop, syringe, dropper, spoon, or colonic irrigation device, may hold the dosage form, for instance cream, foam, gel, lotion, ointment, paste, powder, shake lotion and solid, a measured quantity of composition equaling a full unit dose or fraction thereof (e.g., ½, ⅓, or ¼ of a dose). There may be a single unit dose, or multiple unit doses, in a single dose of administration. The kit may include instructions regarding the size of the unit dose, or fraction thereof.

### III. Pharmaceutical Compositions Comprising HPPs

Another aspect of the disclosure provides a pharmaceutical composition comprising at least one HPP of a parent drug or a related compound thereof that can be used to treat signs, symptoms and/or complications of viral, bacterial, protozoal, and/or fungal infections, and a pharmaceutically acceptable carrier.

A pharmaceutical composition may comprise more than one HPP of different parent drugs. The different parent drugs can belong to the same or different categories of drugs that are used to treat signs, symptoms and/or complications of viral, bacterial, and/or protozoal, and/or fungal infections. For example, a pharmaceutical composition may comprise HPPs of parent drugs or related compounds thereof, the parent drugs being selected from 5-lipoxygenase-activating protein (FLAP) inhibitors, 5-lipoxygenase inhibitors, leukotriene receptor antagonists, aspirin and other nonsteroidal anti-inflammatory drugs (NSAIDs), other anti-inflammatory drugs, and any combinations thereof.

In certain embodiments, a pharmaceutical composition comprises HPPs of parent drugs or related compounds thereof, the parent drugs being selected from compounds of Structure LRA-1, Structure LRA-2, Structure LRA-3, Structure LRA-4, Structure LRA-5, Structure LRA-6, Structure ARA-1, Structure ARA-2, Structure ARA-3, Structure ARA-4, Structure ARA-5, Structure ARA-6, Structure ARA-7, Structure ARA-8, Structure ARA-9, Structure ARA-11, Structure ARA-11, Structure ARA-12, Structure ARA-13, Structure ARA-14, Structure 5-LI-1, Structure 5-LI-2, Structure 5-LI-3, Structure 5-LI-4, Structure 5-LI-5, Structure 5-LI-6, Structure 5-LI-7, Structure 5-LI-8, Structure FLAP-1, Structure FLAP-2, Structure FLAP-3, Structure FLAP-4, Structure FLAP-5, Structure FLAP-6, Structure NSAID-1, Structure NSAID-2, Structure NSAID-3, Structure NSAID-4, Structure NSAID-5, Structure NSAID-6, Structure NSAID-7, Structure NSAID-8, Structure NSAID-9, Structure NSAID-10, Structure NSAID-11, Structure NSAID-12, and Structure NSAID-13.

The term “pharmaceutically acceptable carrier” as used herein means a pharmaceutically-acceptable material, composition or vehicle, such as a liquid or solid filler, diluent, excipient, solvent or encapsulating material, involved in carrying or transporting an HPP from one location, body fluid, tissue, organ (interior or exterior), or portion of the body, to another location, body fluid, tissue, organ, or portion of the body.

Each carrier is “pharmaceutically acceptable” in the sense of being compatible with the other ingredients, e.g., an HPP, of the formulation and suitable for use in contact with the tissue or organ of a biological subject without excessive toxicity, irritation, allergic response, immunogenicity, or other problems or complications, commensurate with a reasonable benefit/risk ratio.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) alcohol, such as ethyl alcohol and n-propyl or isopropyl alcohol; (20) phosphate buffer solutions; and (21) other non-toxic compatible substances employed in pharmaceutical formulations, such as acetone.

The pharmaceutical compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

In one embodiment, the pharmaceutically acceptable carrier is an aqueous carrier, e.g. buffered saline and the like. In certain embodiments, the pharmaceutically acceptable carrier is a polar solvent, e.g. acetone and ethyl alcohol. In certain embodiments, the pharmaceutically acceptable carrier is an aqueous solution containing 10-35% ethanol by volume.

The concentration of HPP in these formulations can vary widely, and will be selected primarily based on fluid volumes, viscosities, body weight and the like in accordance with the particular mode of administration selected and the biological subject's needs. For example, the concentration can be 0.001% to 50%, 0.01% to 30%, 0.1% to 20%, or 1% to 10% wt.

The compositions of the invention can be administered for prophylactic, therapeutic, and/or hygienic use. Such administration can be topical, mucosal, e.g., oral, nasal, vaginal, rectal, parenteral, transdermal, subcutaneous, intramuscular, intravenous, via inhalation, ophthalmic and other convenient routes. The pharmaceutical compositions can be administered in a variety of unit dosage forms depending upon the method of administration. For example, unit dosage forms suitable for oral administration include powder, tablets, pills, capsules and lozenges and for transdermal administration include solution, suspension and emulsion.

Thus, a typical pharmaceutical composition for transdermal, oral, and intravenous administrations would be about 0.001 g to about 100 g, about 0.01 g to about 10 g, or about 0.1 g to about 1 g per subject per day. Dosages from about 0.00001 mg, up to about 100 g, per subject per day may be used. Actual methods for preparing parenterally administrable compositions will be known or apparent to those skilled in the art and are described in more detail in such publications as Remington: The Science and Practice of Pharmacy 21st ed., Lippincott Williams & Wilkins, (2005).

### IV. Applications of HPPs

### i) Methods for Penetrating a Biological Barrier.

Another aspect of the disclosure provides a method of delivering a drug molecule to a site within a biological subject by penetrating one or more biological barriers in the biological subject. The method comprises administering to a biological subject an HPP or a pharmaceutical composition thereof. In certain embodiments, an HPP exhibits more than about 20 times or higher, 50 times or higher, about 100 times or higher, about 200 time higher, about 300 times or higher, about 500 times or higher, about 1,000 times or higher penetration rate through one or more biological barriers than its parent drug.

The term “biological barrier” as used herein refers to a biological layer that separates an environment, preferably an in vivo environment, into different spatial areas or compartments, which separation is capable of modulating (e.g. restricting, limiting, enhancing or taking no action in) the passing through, penetrating or translocation of substance or matter from one compartment/area to another. The different spatial areas or compartments as referred to herein may have the same or different chemical or biological environment(s). The biological layer as referred herein includes, but is not limited to, a biological membrane, a cell layer, a biological structure, an inner surface of subjects, organisms, organs or body cavities, an external surface of subjects, organisms, organs or body cavities, or any combination or plurality thereof.

Examples of a biological membrane include a lipid bilayer structure, eukaryotic cell membrane, prokaryotic cell membrane, and intracellular membrane (e.g., nucleus or organelle membrane, such as membrane or envelope of Golgi apparatus, rough and smooth endoplasmic reticulum (ER), ribosomes, vacuoles, vesicles, liposomes, mitochondria, lysosome, nucleus, chloroplasts, plastids, peroxisomes or microbodies).

The lipid bilayer referred to herein is a double layer of lipid-class molecules, including, but not limited to, phospholipids and cholesterol. In a particular embodiment, lipids for the bilayer are amphiphilic molecules consisting of polar head groups and non-polar fatty acid tails. The bilayer is composed of two layers of lipids arranged so that their hydrocarbon tails face one another to form an oily core held together by the hydrophobic effect, while their charged heads face the aqueous solutions on either side of the membrane. In another particular embodiment, the lipid bilayer may contain one or more embedded protein and/or sugar molecule(s).

Examples of a cell layer include a lining of eukaryotic cells (e.g., epithelium, lamina propria, smooth muscle or muscularis mucosa (in gastrointestinal tract)), a lining of prokaryotic cells (e.g., surface layer or S-layer which refers to a two-dimensional structure monomolecular layer composed of identical proteins or glycoproteins, specifically, an S-layer refers to a part of a cell envelope commonly found in bacteria and archaea), a biofilm (a structured community of microorganisms encapsulated within a self-developed polymeric matrix and adherent to a living or inert surface), and a plant cell layer (e.g., epidermis). The cells may be normal cells or pathological cells (e.g. disease cells, cancer cells).

Examples of biological structures include structures sealed by tight or occluding junctions that provide a barrier to the entry of toxins, bacteria and viruses, e.g. the blood-milk barrier and the blood-brain barrier (BBB). In particular, BBB is composed of an impermeable class of endothelium, which presents both a physical barrier through tight junctions adjoining neighboring endothelial cells and a transport barrier comprised of efflux transporters. The biological structure may also include a mixture of cells, proteins and sugars (e.g., blood clots).

Examples of the inner surface of subjects, organisms, organs or body cavities include buccal mucosa, esophageal mucosa, gastric mucosa, intestinal mucosa, olfactory mucosa, oral mucosa, bronchial mucosa, uterine mucosa and endometrium (the mucosa of the uterus), inner layer of the wall of a pollen grain or the inner wall layer of a spore, or a combination or plurality thereof.

Examples of the external surface of subjects, organisms, organs or body cavities include capillaries (e.g. capillaries in the heart tissue), mucous membranes that are continuous with skin (e.g. such as at the nostrils, the lips, the ears, the genital area, and the anus), outer surface of an organ (e.g. liver, lung, stomach, brain, kidney, heart, ear, eye, nose, mouth, tongue, colon, pancreas, gallbladder, duodenum, rectum stomach, colonrectum, intestine, vein, respiratory system, vascular system, anorectum and pruritus ani), skin, cuticle (e.g. dead layers of epidermal cells or keratinocytes or superficial layer of overlapping cells covering the hair shaft of an animal, a multi-layered structure outside the epidermis of many invertebrates, plant cuticles or polymers cutin and/or cutan, external layer of the wall of a pollen grain or the external wall layer of a spore), and a combination or plurality thereof.

In addition, a biological barrier further includes a sugar layer, a protein layer or any other biological layer, or a combination or plurality thereof. For example, skin is a biological barrier that has a plurality of biological layers. A skin comprises an epidermis layer (outer surface), a dermis layer and a subcutaneous layer. The epidermis layer contains several layers including a basal cell layer, a spinous cell layer, a granular cell layer, and a stratum corneum. The cells in the epidermis are called keratinocytes. The stratum corneum (“horny layer”) is the outmost layer of the epidermis, wherein cells here are flat and scale-like (“squamous”) in shape. These cells contain a lot of keratin and are arranged in overlapping layers that impart a tough and oilproof and waterproof character to the skin's surface.

ii). Methods of Using HPPs and Pharmaceutical Compositions Thereof in Treatment of Signs, Symptoms and/or Complications of Viral, Bacterial, Protozoal, and/or Fungal Infections and Related Conditions

Another aspect of the disclosure provides a method of treating a sign, symptom and/or complication of viral, bacterial, protozoal, and/or fungal infections and related conditions in a subject by administrating an HPP or a pharmaceutical composition thereof to the subject.

The term “treating” as used herein means curing, alleviating, inhibiting, or preventing. The term “treat” as used herein means cure, alleviate, inhibit, or prevent. The term “treatment” as used herein means cure, alleviation, inhibition or prevention.

The term “biological subject,” or “subject” as used herein means an organ, a group of organs that work together to perform a certain task, an organism, or a group of organisms. The term “organism” as used herein means an assembly of molecules that function as a more or less stable whole and has the properties of life, such as animal, plant, fungus, or micro-organism.

The term “animal” as used herein means a eukaryotic organism characterized by voluntary movement. Examples of animals include, without limitation, vertebrata (e.g. human, mammals, birds, reptiles, amphibians, fishes, marsipobranchiata and leptocardia), tunicata (e.g. thaliacea, appendicularia, sorberacea and ascidioidea), articulata (e.g. insecta, myriapoda, malacapoda, arachnida, pycnogonida, merostomata, crustacea and annelida), gehyrea (anarthropoda), and helminthes (e.g. rotifera).

In certain embodiments, a method of treating a sign, symptom and/or complication of a viral, bacterial, protozoal, and/or fungal infection and related conditions in a subject comprises administering a therapeutic effective amount of an HPP, or a pharmaceutical composition thereof to the subject.

An HPP or a pharmaceutical composition thereof can be administered to a biological subject by any administration route known in the art, including without limitation, oral, enteral, buccal, nasal, topical, rectal, vaginal, aerosol, transmucosal, epidermal, transdermal, dermal, ophthalmic, pulmonary, subcutaneous, and/or parenteral administration. In some embodiments, preferably, an HPP or a pharmaceutical composition thereof is administered transdermally or topically to the subject. The pharmaceutical compositions can be administered in a variety of unit dosage forms depending upon the method of administration.

An HPP or a pharmaceutical composition thereof can be given to a subject in the form of formulations or preparations suitable for each administration route. The formulations useful in the methods of the invention include one or more HPPs, one or more pharmaceutically acceptable carriers therefor, and optionally other therapeutic ingredients. The formulations may conveniently be presented in unit dosage form and may be prepared by any methods well known in the art of pharmacy. The amount of active ingredient which can be combined with a carrier material to produce a single dosage form will vary depending upon the subject being treated and the particular mode of administration. The amount of an HPP which can be combined with a carrier material to produce a pharmaceutically effective dose will generally be that amount of an HPP which produces a therapeutic effect. Generally, the amount of the HPP will range from about 1 percent to about ninety-nine percent of the HPP, sometimes preferably from about 1 percent to about 20 percent, by weight.

Methods of preparing these formulations or compositions include the step of bringing into association an HPP with one or more pharmaceutically acceptable carriers and, optionally, one or more accessory ingredients. In general, the formulations are prepared by uniformly and intimately bringing into association an HPP with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an HPP as an active ingredient. A compound may also be administered as a bolus, electuary, or paste.

In solid dosage forms for oral administration (e. g., capsules, tablets, pills, dragees, powders, granules and the like), the HPP is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate, (5) solution retarding agents, such as paraffin, (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered HPPs and an inert liquid diluent. Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of an HPP therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain pacifying agents and may be of a composition that they release the HPP(s) only, or preferentially, in a certain portion of the gastrointestinal tract, optionally, in a delayed manner. Examples of embedding compositions which can be used include polymeric substances and waxes. The HPP can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the HPP, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the HPP, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more HPPs with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent. Formulations which are suitable for vaginal administration also include pessaries, tampons, creams, gels, pastes, foams or spray formulations containing such carriers as are known in the art to be appropriate.

Formulations for the topical or transdermal or epidermal or dermal administration of an HPP composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required. The ointments, pastes, creams and gels may contain, in addition to the HPP composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to the HPP composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. The best formulations for the topical or transdermal administration are solution, sometimes preferably aqueous solution, such as an aqueous containing ethanol or isopropanol.

An HPP or a pharmaceutical composition thereof can be alternatively administered by aerosol. This can be accomplished by preparing an aqueous aerosol, liposomal preparation or solid particles containing the HPPs. A nonaqueous (e.g., fluorocarbon propellant) suspension could be used. Sonic nebulizers can also be used. An aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (e.g., Tweens™, Pluronics™, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches can also be used to deliver HPP compositions to a target site. Such formulations can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Formulations suitable for parenteral administration comprise an HPP in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacterostats, solutes which render the formulation isotonic with the blood of the intended recipient or suspending or thickening agents.

Examples of suitable aqueous and nonaqueous carriers which may be employed in the formulations suitable for parenteral administration include water, ethanol, polyols (e.g., such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Formulations suitable for parenteral administration may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Injectable depot forms are made by forming microencapsule matrices of an HPP or in biodegradable polymers such as polylactide-polyglycolide. Depending on the ratio of the HPP to polymer, and the nature of the particular polymer employed, the rate of drug release can be controlled. Examples of other biodegradable polymers include poly (orthoesters) and poly(anhydrides). Depot injectable formulations are also prepared by entrapping the HPP in liposomes or microemulsions which are compatible with body tissue.

In certain embodiments, an HPP or a pharmaceutical composition thereof is delivered to an action site in a therapeutically effective dose. As is known in the art of pharmacology, the precise amount of the pharmaceutically effective dose of an HPP that will yield the most effective results in terms of efficacy of treatment in a given patient will depend upon, for example, the activity, the particular nature, pharmacokinetics, pharmacodynamics, and bioavailability of a particular HPP, physiological condition of the subject (including race, age, sex, weight, diet, disease type and stage, general physical condition, responsiveness to a given dosage and type of medication), the nature of pharmaceutically acceptable carriers in a formulation, the route and frequency of administration being used, and the severity or propensity of the condition that is to be treated. However, the above guidelines can be used as the basis for fine-tuning the treatment, e.g., determining the optimum dose of administration, which will require no more than routine experimentation consisting of monitoring the subject and adjusting the dosage. Remington: The Science and Practice of Pharmacy (Gennaro ed. 20.sup.th edition, Williams & Wilkins PA, USA) (2000).

iii). Use of an HPP and Pharmaceutical Compositions Thereof in the Manufacture of a Medicament for Treatment of Signs, Symptoms and Complications of Viruses and/or Bacteria and/or Protozoa, and/or Fungi Infections and Related Conditions

Another aspect of the disclosure provides use of an HPP and pharmaceutical compositions thereof in the manufacture of a medicament for treatment of signs, symptoms and complications of viruses infection, bacteria infection, protozoa infection and/or fungi infection and related conditions.

In some embodiments, the present disclosure provides use of
(1) (pyrrolidin-2-yl)methyl 2-(2-(2,6-dichlorophenylamino)phenyl) acetate hydrochloride;
(2) 2-(diethylamino)ethyl 2-(4-isobutylphenyl) propanoate hydrochloride;
(3) 2-(N,N-diethylamino)ethyl 2-[1-[[(1R)-1-[3-[2-(7-chloroquinolin-2-yl) ethenyl]phenyl]-3-[2-(2-hydroxypropan-2-yl)phenyl]propyl]sulfanylmethyl]cyclopropyl]acetate hydrochloride;
(4) 2-(diethylamino)ethyl acetoxybenzoate hydrochloride;
(5) (RS)—N-[1-(1-benzothien-2-yl)ethyl]-N-(2-N,N-diethylaminoacetyloxy) urea hydrochloride;
(6) (pyrrolidin-2-yl)methyl 2-cyclopentyl-2-[4-(quinolin-2-ylmethoxy)phenyl]acetate hydrochloride;
(7) (pyrrolidin-2-yl)methyl 3-[[3-[(E)-2-(7-chloroquinolin-2-yl) ethenyl]phenyl]-[3-(dimethylamino)-3-oxopropyl]sulfanylmethyl]sulfanylpropanoate hydrochloride;
(8) (Z)-2-(N,N-diethylaminoethyl) ethyl 2-(5-fluoro-2-methyl-1-(4-methylsulfinyl)benzylidene)-1H-inden-1-yl) acetate AcOH;
(9) 2-(dimethylamino)ethyl 2-(3-phenoxyphenyl) propionate hydrochloride,
(10)S-(2-(dimethylamino)ethyl 2-(3-phenoxyphenyl) propanethioate hydrochloride;
(11) 2-(dipropylamino)ethyl 4-acetoxy-2′,4′-difluoro-[1,1′-biphenyl]-3-carboxylate hydrochloride[2-(dipropylamino)ethyl 5-(2,4-difluorophenyl) acetylsalicylate hydrochloride;
(12) (pyrrolidin-2-yl)methyl 3-[[1-(4-chlorobenzyl)-4-methyl-6-(5-phenylpyridin-2-yl) methoxy]-4,5-dihydro-1H-thiopyrano[2,3,4-c,d]indol-2-yl]-2,2-dimethylpropanoate hydrochloride;
(13) (pyrrolidin-2-yl)methyl 5-benzoyl-2,3-dihydro-1H-pyrrolizine-1-carboxylate hydrochloride;
(14) (pyrrolidin-2-yl)methyl 1,8-diethyl-1,3,4,9-tetrahydropyrano[3,4-b]indole-1-acetate hydrochloride;
(15) (pyrrolidin-2-yl)methyl 2-(2-fluorobiphenyl-4-yl) propanoate hydrochloride;
(16) (pyrrolidin-2-yl)methyl 2-(6-methoxy-2-naphthyl) propanoate hydrochloride;
(17) (pyrrolidin-2-yl)methyl 2-[(2,3-dimethylphenyl)amino]benzoate hydrochloride;
(18) (pyrrolidin-2-yl)methyl 6-chloro-α-methylcarbazole-2-acetate hydrochloride;
(19) (pyrrolidin-2-yl)methyl 4-[4-[3-(4-acetyl-3-hydroxy-2-propylphenoxy) propylsulfonyl]phenyl]-4-oxo-butanoate hydrochloride;
(20) (pyrrolidin-2-yl)methyl (Z)-5-fluoro-2-methyl-1-[(4-methylsulfinyl)phenylmethylene]-1H-indene-3-acetate hydrochloride;
(21) (pyrrolidin-2-yl)methyl 1-methyl-5-(4-methylbenzoyl)-1H-pyrrole-2-acetate hydrochloride;
(22) (pyrrolidin-2-yl)methyl 1-(4-chlorobenzoyl)-5-methoxy-2-methylindole 3-acetate hydrochloride;
(23) (pyrrolidin-2-yl)methyl 5-(4-chlorobenzoyl)-1,4-dimethyl-1H-pyrrole-2-acetate hydrochloride;
(24) (pyrrolidin-2-yl)methyl 3-chloro-4-(2-propenyloxy)benzeneacetate hydrochloride;
(25) (pyrrolidin-2-yl)methyl 2-(4-isobutylphenyl) propanoate hydrochloride;
(26) (pyrrolidin-2-yl)methyl 2-acetoxybenzoate hydrochloride;
(27) (pyrrolidin-2-yl)methyl 5-(2,4-difluorophenyl) salicylate hydrochloride;
(28) (pyrrolidin-2-yl)methyl 5-(2,4-difluorophenyl) acetylsalicylate hydrochloride;
(29) (pyrrolidin-2-yl)methyl 2-(3-benzoylphenyl) propionate hydrochloride;
(30) (pyrrolidin-2-yl)methyl 2-(3-phenoxyphenyl) propionate hydrochloride;
(31) (pyrrolidin-2-yl)methyl (Z)-5-fluoro-2-methyl-1-[(4-methylsulfinyl)phenylmethylene]-1H-indene-3-acetate hydrochloride;
(32) (pyrrolidin-2-yl)methyl 5-(4-chlorobenzoyl)-1,4-dimethyl-1H-pyrrole-2-acetate hydrochloride;
(33) (pyrrolidin-2-yl)methyl α-methyl-4-(2-thienylcarbonyl)benzeneacetate hydrochloride;
(34) (pyrrolidin-2-yl)methyl 6-chloro-α-methyl-9H-carbazole-2-acetate hydrochloride;
(35) (pyrrolidin-2-yl)methyl 2-(4-chlorophenyl)-α-methyl-5-benzoxazoleacetate hydrochloride;
(36) (pyrrolidin-2-yl)methyl 2-[3-chloro-4-(3-pyrrolin-1-yl)phenyl]propanoate hydrochloride;
(37) (pyrrolidin-2-yl)methyl α-methyl-(4-chlorobenzoyl)-5-methoxy-2-methylindole-3-acetate hydrochloride;
(38) (pyrrolidin-2-yl)methyl 2-(5H-[1]-benzopyrano[2,3-b]pyridin-7-yl) propionate hydrochloride;
(39) (pyrrolidin-2-yl)methyl α-methyl-4-[(2-methyl-2-propen-1-yl)amino]benzeneacetate hydrochloride;
(40) (pyrrolidin-2-yl)methyl 5-benzoyl-α-methyl-2-thiopheneacetate hydrochloride;
(41) (pyrrolidin-2-yl)methyl 10,11-dihydro-α-methyl-10-oxodibenzo[b,f]thiepin-2-acetate hydrochloride;
(42) (pyrrolidin-2-yl)methyl 2-(8-methyl-5-oxo-6H-benzo[b][1]benzoxepin-3-yl)propanote hydrochloride;
(43) (pyrrolidin-2-yl)methyl 2-[4-[(2-oxocyclopentyl)methyl]phenyl]propanote hydrochloride;
(44) (pyrrolidin-2-yl)methyl 4-(1,3-dihydro-1-oxo-2H-isoindol-2-yl)-α-methylbenzeneacetate hydrochloride;
(45) (pyrrolidin-2-yl)methyl 2-chloro-2-(3-chloro-4-cyclohexylphenyl) acetate hydrochloride;
(46) (pyrrolidin-2-yl)methyl 4,5-diphenyl-2-oxazolepropanoate hydrochloride;
(47) (pyrrolidin-2-yl)methyl 3-(4-biphenylcarbonyl) propanoate hydrochloride;
(48) (pyrrolidin-2-yl)methyl 3-[5-(4-chlorophenyl) furan-2-yl]-3-hydroxypropanoate hydrochloride;
(49) (pyrrolidin-2-yl)methyl 6-chloro-5-cyclohexyl-1-indancarboxylate hydrochloride;
(50) (pyrrolidin-2-yl)methyl 1-(4-chlorobenzoyl)-5-methoxy-2-methylindole 3-acetate hydrochloride;
(51) (pyrrolidin-2-yl)methyl 1-methyl-5-(4-methylbenzoyl)-1H-pyrrole-2-acetate hydrochloride;
(52) (pyrrolidin-2-yl)methyl 5-(4-chlorobenzoyl)-1,4-dimethyl-1H-pyrrole-2-acetate hydrochloride;
(53) (pyrrolidin-2-yl)methyl 3-chloro-4-(2-propenyloxy)benzeneacetate hydrochloride;
(54) (pyrrolidin-2-yl)methyl 3-2-(2,4-dichlorophenoxy)benzeneacetate hydrochloride;
(55) (pyrrolidin-2-yl)methyl 4-acetamidophenyl salicylate hydrochloride;
(56) (pyrrolidin-2-yl)methyl 2-[(2,3-dimethylphenyl)amino]benzoate hydrochloride;
(57) (pyrrolidin-2-yl)methyl 2-[(2,3-dimethylphenyl)amino]benzoate hydrochloride;
(58) (pyrrolidin-2-yl)methyl 2-[3-(trifluoromethyl)phenyl]amino]-3-pyridinecarboxylate hydrochloride;
(59) (pyrrolidin-2-yl)methyl 2-[(2,6-dichloro-3-methylphenyl)amino]benzoate hydrochloride; and
(60) (pyrrolidin-2-yl)methyl 2-[[2-methyl-3-(trifluoromethyl)phenyl]amino]-3-pyridinecarboxylate hydrochloride, in the manufacture of a medicament for treatment of signs, symptoms and complications of viruses infections, bacteria infections, protozoa infections and/or fungi infections and related conditions.

In some embodiments, the present disclosure provides use of 2-(diethylamino)ethyl 2-(4-isobutylphenyl) propanoate hydrochloride, 2-(diethylamino)ethyl 2-acetoxybenzoate hydrochloride, (pyrrolidin-2-yl)methyl 2-(2-(2,6-dichlorophenylamino)phenyl) acetate hydrochloride, (pyrrolidin-2-yl)methyl 2-(6-methoxy-2-naphthyl) propanoate hydrochloride, (pyrrolidin-2-yl)methyl 2-(2-fluorobiphenyl-4-yl) propanoate hydrochloride, (Z)-2-(N,N-diethylaminoethyl) ethyl 2-(5-fluoro-2-methyl-1-(4-methylsulfinyl)benzylidene)-1H-inden-1-yl) acetate ACOH, (pyrrolidin-2-yl)methyl 2-[(2,3-dimethylphenyl)amino]benzoate hydrochloride, (pyrrolidin-2-yl)methyl α-methyl-4-(2-thienylcarbonyl)benzeneacetate hydrochloride, (pyrrolidin-2-yl)methyl 5-(2,4-difluorophenyl) salicylate hydrochloride, or (pyrrolidin-2-yl)methyl 5-(2,4-difluorophenyl) acetylsalicylate hydrochloride in the manufacture of a medicament for treatment of signs, symptoms, and complications of H1N1 virus and other flu viruses infections.

In some embodiments, the present disclosure provides use of 2-(diethylamino)ethyl 2-(4-isobutylphenyl) propanoate hydrochloride, 2-(diethylamino)ethyl 2-acetoxybenzoate hydrochloride, (pyrrolidin-2-yl)methyl 2-(2-(2,6-dichlorophenylamino)phenyl) acetate hydrochloride, (pyrrolidin-2-yl)methyl 2-(6-methoxy-2-naphthyl) propanoate hydrochloride, (pyrrolidin-2-yl)methyl 5-benzoyl-2,3-dihydro-1H-pyrrolizine-1-carboxylate hydrochloride, (pyrrolidin-2-yl)methyl 1,8-Diethyl-1,3,4,9-tetrahydropyrano[3,4-b]indole-1-acetate hydrochloride, (pyrrolidin-2-yl)methyl 2-[3-chloro-4-(3-pyrrolin-1-yl)phenyl]propanoate hydrochloride, (pyrrolidin-2-yl)methyl 2-(5H-[1]-benzopyrano[2,3-b]pyridin-7-yl) propionate hydrochloride, pyrrolidin-2-yl)methyl 4,5-diphenyl-2-oxazolepropanoate hydrochloride, or (pyrrolidin-2-yl)methyl 2-(3-benzoylphenyl) propionate hydrochloride in the manufacture of a medicament for treatment of signs, symptoms, and complications of COVID-19 and other coronaviruses infections.

In some embodiments, the present disclosure provides use of 2-(diethylamino)ethyl 2-(4-isobutylphenyl) propanoate hydrochloride, 2-(diethylamino)ethyl 2-acetoxybenzoate hydrochloride, (pyrrolidin-2-yl)methyl 2-(2-(2,6-dichlorophenylamino)phenyl) acetate hydrochloride, (pyrrolidin-2-yl)methyl 2-(6-methoxy-2-naphthyl) propanoate hydrochloride, (pyrrolidin-2-yl)methyl 5-benzoyl-2,3-dihydro-1H-pyrrolizine-1-carboxylate hydrochloride, (pyrrolidin-2-yl)methyl 1,8-Diethyl-1,3,4,9-tetrahydropyrano[3,4-b]indole-1-acetate hydrochloride, (pyrrolidin-2-yl)methyl 2-[3-chloro-4-(3-pyrrolin-1-yl)phenyl]propanoate hydrochloride, (pyrrolidin-2-yl)methyl 2-(5H-[1]-benzopyrano[2,3-b]pyridin-7-yl) propionate hydrochloride, pyrrolidin-2-yl)methyl 4,5-diphenyl-2-oxazolepropanoate hydrochloride, or (pyrrolidin-2-yl)methyl 2-(3-benzoylphenyl) propionate hydrochloride in the manufacture of a medicament for treatment of signs, symptoms, and complications of sepsis.

In some embodiments, the present disclosure provides use of 2-(diethylamino)ethyl 2-acetoxybenzoate hydrochloride, (pyrrolidin-2-yl)methyl 2-(2-(2,6-dichlorophenylamino)phenyl) acetate hydrochloride, (pyrrolidin-2-yl)methyl 2-(3-phenoxyphenyl) propionate hydrochloride, 2-(diethylamino)ethyl 2-(4-isobutylphenyl) propanoate hydrochloride, (pyrrolidin-2-yl)methyl 2-[[2-[(2,6-dichlorophenyl)amino]phenyl]acetoxy]acetate hydrochloride, (pyrrolidin-2-yl)methyl 1-methyl-5-(4-methylbenzoyl)-1H-pyrrole-2-acetate hydrochloride, (pyrrolidin-2-yl)methyl 5-(4-chlorobenzoyl)-1,4-dimethyl-1H-pyrrole-2-acetate hydrochloride, or (pyrrolidin-2-yl)methyl dichlorophenoxy)benzeneacetate hydrochloride in the manufacture of a medicament for treatment of viruses, bacteria, protozoa, and fungi infections-induced sepsis.

In some embodiments, the present disclosure provides use of 2-(diethylamino)ethyl 2-acetoxybenzoate hydrochloride, (pyrrolidin-2-yl)methyl 2-(6-methoxy-2-naphthyl) propanoate hydrochloride, 2-(diethylamino)ethyl 2-(4-isobutylphenyl) propanoate hydrochloride, (pyrrolidin-2-yl)methyl 5-benzoyl-2,3-dihydro-1H-pyrrolizine-1-carboxylate hydrochloride, or (pyrrolidin-2-yl)methyl 1,8-diethyl-1,3,4,9-tetrahydropyrano[3,4-b]indole-1-acetate hydrochloride in the manufacture of a medicament for treatment of signs, symptoms and complications of drug-resistant viruses, bacteria, fungi, and protozoa infections.

V. ADVANTAGES

Aspirin, other NSAIDs, and other anti-inflammatory drugs may cause nausea, dyspepsia, heartburn, vomiting, gastroduodenal bleeding, gastric ulcerations and gastritis. Gastroduodenal bleeding induced by NSAIDs is generally painless but can lead to fecal blood loss and may cause persistent iron deficiency anemia.

By transdermal administration, the high penetration prodrug of Aspirin, other NSAIDs, and other anti-inflammatory drugs, or a composition thereof, can deliver relatively high tissue concentrations of drugs (to maximize the efficacy) while maintaining low plasma concentrations of the drugs (to minimize the side effects), resulting in improved efficacy and reduced adverse effects compared to orally administered NSAIDs and other anti-inflammatory drugs.

The current NSAIDs and other anti-inflammatory drugs cannot penetrate scar tissues (fibroids) of inflammatory lung, liver, heart, respiratory system, kidney, stomach, bowel, uterine, and other tissues significantly and cannot relieve the inflammation of these organs and tissues. In contrast, the high penetration pro-drugs of aspirin, other NSAIDs, and other antiinflammatory drugs herein disclosed can penetrate any biological barriers and can relieve or stop inflammation in any organ and tissues.

### VI. EXAMPLES

The following examples are provided to better illustrate the claimed invention and are not to be interpreted in any way as limiting the scope of the invention. All specific compositions, materials, and methods described below, in whole or in part, fall within the scope of the invention. These specific compositions, materials, and methods merely illustrate specific embodiments falling within the scope of the invention. Based on the present disclosure, one skilled in the art may develop equivalent compositions, materials, and methods without the exercise of inventive capacity and without departing from the scope of the invention. It will be understood that many variations can be made in the procedures herein described while remaining within the bounds of the invention. All such variations are included within the scope of the invention.

In certain embodiments, a parent compound having the following Structure F—C:

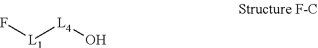

is converted to an HPP having Structure L-1:

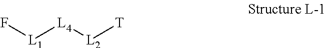

including stereoisomers and pharmaceutically acceptable salts thereof, wherein: F, L1, L2, and L4 are defined as supra;
T is a transportational unit of an HPP. For example, T is selected from W and R₆ as defined supra.

In certain embodiments of the invention, an HPP having Structure L-1 is prepared according to organic synthesis by reacting the parent compounds or derivatives of the parent compounds having Structure D(e.g. acid halides, mixed anhydrides of the parent compounds, etc.):

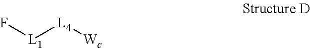

with compounds of Structure E (Scheme 1):

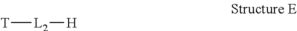

wherein W_{C} is selected from OH, halogen, alkoxycarbonyl and substituted aryloxycarbonyloxy; and
F, L₁, L₂, L₄ and T are defined as supra.

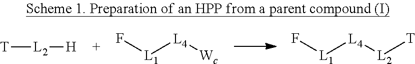

In certain embodiments, an HPP having Structure L-1 is prepared following Scheme 1 as described supra, wherein L₄ is C═O.

In certain embodiments, a parent compound having the following Structure F—N:

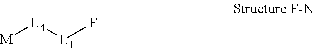

reacts with a compound having the following structure G:

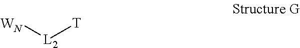

to obtain an HPP of Structure L-1:

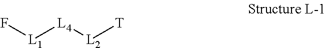

including stereoisomers and pharmaceutically acceptable salts thereof, wherein:
F, L1, L2, and L4 are defined as supra;
T is a transportational unit of an HPP. For example, T is selected from W and R₆ as defined supra; and
M is selected from Na, K, or other metal. W_{N} is selected from OH, halogen, alkoxycarbonyl and substituted aryloxycarbonyloxy. (Scheme 2)

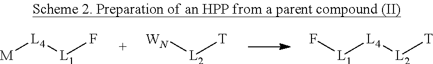

In certain embodiments, an HPP having a structure of Structure L-1 is prepared by organic synthesis wherein the unwanted reactive sites such as —C(═O) OH, —NH₂, —OH, or —SH are protected before linking a transportational unit with a functional unit according to one of the synthetic route as described supra. In certain embodiments, the obtained protected HPP may be further partially or completely deprotected to render a partially protected HPP or an unprotected HPP respectively.

### Example 1. Preparation of (Pyrrolidin-2-Yl) Methyl 2-(2-(2,6-Dichlorophenylamino)Phenyl) Acetate·Hydrochloride

2-(2-(2,6-Dichlorophenylamino)phenyl) acetic acid (Diclofenac, 29.6 g) and N-Boc-L-prolinol (tert-butoxycarbonyl-2-pyrrolidinemethanol, 20.1 g) were put into 1 L round-bottom flask, aceton (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (85% yield).

### Example 2. Preparation of 2-(Diethylamino)Ethyl 2-(4-Isobutylphenyl) Propanoate Hydrochloride

2-(4-Isobutylphenyl) propionyl chloride (22.5 g) was dissolved in chloroform (100 ml). The mixture was cooled to 0° C. Triethylamine (15 ml) and diethylaminoethanol (11.7 g) were added into the reaction mixture. The mixture is stirred for 3 hours at RT. The solid side product was removed by filtration and washed with chloroform (3×30 ml). 1N HCl in ethyl acetate (100 ml) was added, the solid was collected and washed with ethyl acetate (5×50 ml) and dried in vacuum oven at 40° C. (92% yield).

### Example 3. Preparation of 2-(N,N-Diethylamino)Ethyl 2-[1-[[(1R)-1-[3-[2-(7-Chloroquinolin-2-Yl) Ethenyl]Phenyl]-3-[2-(2-Hydroxypropan-2-Yl)Phenyl]Propyl]Sulfanylmethyl/Cyclopropyl]Acetate·Hydrochloride

2-[1-[[(1R)-1-[3-[2-(7-chloroquinolin-2-yl) ethenyl]phenyl]-3-[2-(2-hydroxypropan-2-yl)phenyl]propyl]sulfanylmethyl]cyclopropyl]acetic acid (Montelukast, 58.6 g) and 2-(N,N-diethylamino) ethano (12 g) were put into 1 L round-bottom flask, aceton (200 ml) was added into the mixture. 1-Ethyl-(3-dimethylaminopropyl) carbodiimide·hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 0.5N HCl in ethyl acetate (200 ml) was added, the mixture was stirred for 3 hours. The solid was collected and washed with ethyl acetate (5×50 ml) and dried in vacuum oven at 40° C. (78% yield).

### Example 4. Preparation of 2-(Diethylamino)Ethyl 2-Acetoxybenzoate Hydrochloride

Acetylsalicyloyl chloride (1-(chlorocarbonyl) phenyl acetate, 19.8 g) was dissolved in chloroform (100 ml). The mixture was cooled to 0° C. Trimethylamine (15 ml) and diethylaminoethanol (11.7 g) were added into the reaction mixture. The mixture is stirred for 3 hours at RT. The solid side product was removed by filtration and washed with chloroform (3×30 ml). the solid was collected and washed with ethyl acetate (5×50 ml) and dried in vacuum oven at 40° C. (81% yield).

### Example 5. Preparation of (RS)—N-[1-(1-Benzothien-2-Yl)Ethyl]-N-(2-N,N-Diethylaminoacetyloxy) Urea·Hydrochloride

2-(N,N-diethylamino) acetic acid (13.1 g) and (+)—N-hydroxy-N-(1-benzo[b]thien-2-ylethyl) urea (Zileuton, 23.6 g) were put into 1 L round-bottom flask, aceton (200 ml) was added into the mixture. 1-Ethyl-(3-dimethylaminopropyl) carbodiimide·hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 0.5N HCl in ethyl acetate (200 ml) was added, the mixture was stirred for 3 hours. The solid was collected and washed with ethyl acetate (5×50 ml) and dried in vacuum oven at 40° C. (76% yield).

### Example 6. Preparation of (Pyrrolidin-2-Yl) Methyl 2-Cyclopentyl-2-[4-(Quinolin-2-Ylmethoxy)Phenyl]Acetate·Hydrochloride

2-Cyclopentyl-2-[4-(quinolin-2-ylmethoxy)phenyl]acetic acid (Veliflapon, 36.1 g) and N-Boc-L-prolinol (tert-butoxycarbonyl-2-pyrrolidinemethanol, 20.1 g) were put into 1 L round-bottom flask, aceton (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (78% yield).

### Example 7. Preparation of (Pyrrolidin-2-Yl) Methyl 3-[[3-[(E)-2-(7-Chloroquinolin-2-Yl)Ethenyl|Phenyl]-[3-(Dimethylamino)-3-Oxopropyl]Sulfanylmethyl/Sulfanylpropanoate·Hydrochloride

3-[3-[(E)-2-(7-chloroquinolin-2-yl) ethenyl]phenyl]-[3-(dimethylamino)-3-oxopropyl]sulfanylmethyl]sulfanylpropanoic acid (Verlukast, 51.5 g) and N-Boc-L-prolinol (tert-butoxycarbonyl-2-pyrrolidinemethanol, 20.1 g) were put into 1 L round-bottom flask, aceton (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (83% yield).

### Example 8. Preparation of (Z)-2-(N,N-Diethylaminoethyl) Ethyl 2-(5-Fluoro-2-Methyl-1-(4-Methylsulfinyl)Benzylidene)-1H-Inden-1-Yl)Acetate·AcOH

Diethylaminoethanol (11.7 g, 0.1 mol) was dissolved in 10% sodium bicarbonate (200 ml) and acetone (100 ml). (Z)-5-fluoro-2-methyl-1-[(4-methylsulfinyl)phenylmethylene]-1H-indene-3-acetyl chloride (Sulindac chloride, 37.5 g, 0.1 mol) was added into the reaction mixture. The mixture is stirred for 3 hours at RT. The solvents are evaporated off. The residue is suspended in ethyl acetate (500 ml). 5% sodium bicarbonate (200 ml) is added into the reaction mixture with stirring. Ethyl acetate layer is collected and washed with water (3×500 ml). The ethyl acetate solution was dried over anhydrous sodium sulfate. Sodium sulfate is removed by filtration. 6 g of acetic acid is added into the reaction mixture with stirring. The organic solution was evaporated off.

### Example 9. Preparation of 2-(Dimethylamino)Ethyl 2-(3-Phenoxyphenyl) Propionate·Hydrochloride

2-(3-Phenoxyphenyl) propionyl chloride (26.1 g, 0.1 mol) was dissolved in ethyl acetate (300 ml). The mixture was cooled to 0° C. Dimethylaminoethanol (8.9 g) were added into the reaction mixture. Sodium bicarbonate (30 g) is added into the mixture. The mixture is stirred for 5 hours at RT. The mixture is washed with water (3×200 ml). The ethyl acetate solution is dried over anhydrous sodium sulfate. HCl gas (5 g) is bubbled into the mixture. The solid is collected by filtration and washed with ethyl acetate.

### Example 10. Preparation of S-(2-(Dimethylamino)Ethyl 2-(3-Phenoxyphenyl) Propanethioate Hydrochloride

Dimethylaminoethyl mercaptan (10.4 g, 0.1 mol) was dissolved in 10% sodium bicarbonate (200 ml) and acetone (100 ml). 2-(3-Phenoxyphenyl) propionyl chloride (27.3 g, 0.1 mol) was added into the reaction mixture. The mixture is stirred for 3 hours at RT. The solvents are evaporated off. The residue is suspended in ethyl acetate (500 ml). 5% sodium bicarbonate (200 ml) is added into the reaction mixture with stirring. Ethyl acetate layer is collected and washed with water (3×500 ml). The ethyl acetate solution was dried over anhydrous sodium sulfate. Sodium sulfate is removed by filtration. Anhydrous HCl gas (5 g) is bubbled into the reaction mixture with stirring. The solid is collected and washed with ethyl acetate.

### Example 11. Preparation of 2-(Dipropylamino)Ethyl 4-Acetoxy-2′,4′-Difluoro-[1,1′-Biphenyl]-3-Carboxylate Hydrochloride [2-(Dipropylamino)Ethyl 5-(2,4-Difluorophenyl) Acetylsalicylate Hydrochloride]

5-(2,4-Difluorophenyl) acetylsalicyl chloride (31.1 g, 0.1 ml) was dissolved in ethyl acetate (300 ml). The mixture was cooled to 0° C. Diethylaminoethanol (11.7 g, 0.1 mol) were added into the reaction mixture. Then sodium bicarbonate (30 g) is added into the reaction mixture. The mixture is stirred for 3 hours at RT. Then water (200 ml) is added into the mixture. The ethyl acetate layer is collected and washed with water (3×). The solution is dried over anhydrous sodium sulfate. Anhydrous HCl gas is bubbled into the reaction mixture with stirring. The solid is collected and washed with ethyl acetate.

### Example 12. Preparation of (Pyrrolidin-2-Yl) Methyl 3-[[1-(4-Chlorobenzyl)-4-Methyl-6-(5-Phenylpyridin-2-Yl) Methoxy]-4,5-Dihydro-1H-Thiopyrano[2,3,4-c,d]Indol-2-Yl]-2,2-Dimethylpropanoate Hydrochloride

3-[[1-(4-Chlorobenzyl)-4-methyl-6-(5-phenylpyridin-2-yl) methoxy]-4,5-dihydro-1H-thiopyrano[2,3,4-c,d]indol-2-yl]-2,2-dimethylpropanoic acid (59.5 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (77% yield).

### Example 13. Preparation of (Pyrrolidin-2-Yl) Methyl 5-Benzoyl-2,3-Dihydro-1H-Pyrrolizine-1-Carboxylate Hydrochloride

5-Benzoyl-2,3-dihydro-1H-pyrrolizine-1-carboxylic acid (Ketorolac, 25.5 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (77% yield).

### Example 14. Preparation of (pyrrolidin-2-yl)methyl 1,8-Diethyl-1,3,4,9-tetrahydropyrano[3,4-b]indole-1-acetate hydrochloride

1,8-Diethyl-1,3,4,9-tetrahydropyrano[3,4-b]indole-1-acetic acid (Etodolac, 28.7 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (77% yield).

### Example 15. Preparation of (Pyrrolidin-2-Yl) Methyl 2-(2-Fluorobiphenyl-4-Yl) Propanoate Hydrochloride

2-(2-Fluorobiphenyl-4-yl) propionic acid (Flurbiprofen, 24.4 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (87% yield).

### Example 16. Preparation of (Pyrrolidin-2-Yl) Methyl 2-(6-Methoxy-2-Naphthyl) Propanoate Hydrochloride

2-(6-Methoxy-2-naphthyl) propionic acid (Naproxen, 23.0 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-19.2 dimethylaminopropyl) carbodiimide hydrochloride (EDCI, g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (91% yield).

### Example 17. Preparation of (Pyrrolidin-2-Yl) Methyl 2-1 (2,3-Dimethylphenyl)Amino]Benzoate Hydrochloride

2-[(2,3-Dimethylphenyl)amino]benzoic acid (Mefenamic acid, 24.1 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (77% yield).

### Example 18. Preparation of (Pyrrolidin-2-Yl) Methyl 6-Chloro-α-Methylcarbazole-2-Acetate Hydrochloride

6-Chloro-α-methylcarbazole-2-acetic acid (Carprofen, 24.1 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (77% yield).

### Example 19. Preparation of (pyrrolidin-2-yl)methyl 4-[4-[3-(4-acetyl-3-hydroxy-2-propylphenoxy) propylsulfonyl]phenyl]-4-oxo-butanoate hydrochloride

4-[4-[3-(4-Acetyl-3-hydroxy-2-propylphenoxy) propylsulfonyl]phenyl]-4-oxo-butanoic acid (47.6 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40 C (81% yield).

### Example 20. Preparation of (Pyrrolidin-2-Yl) Methyl (Z)-5-Fluoro-2-Methyl-1-[(4-Methylsulfinyl)Phenylmethylene]-1H-Indene-3-Acetate·HCl

(Z)-5-fluoro-2-methyl-1-[(4-methylsulfinyl)phenylmethylene]-1H-indene-3-acetic acid (29.0 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (81% yield).

### Example 21. Preparation of (Pyrrolidin-2-Yl) Methyl 1-Methyl-5-(4-Methylbenzoyl)-1H-Pyrrole-2-Acetate·HCl

1-Methyl-5-(4-methylbenzoyl)-1H-pyrrole-2-acetic acid (26.8 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-4-dimethylaminopropyl) carbodiimide hydrochloride (EDCI, 19.2 g), dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (79% yield).

### Example 22. Preparation of (Pyrrolidin-2-Yl) Methyl 1-(4-Chlorobenzoyl)-5-Methoxy-2-Methylindole 3-Acetate Hydrochloride

1-(4-Chlorobenzoyl)-5-methoxy-2-methylindole 3-acetic acid (29.0 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (85% yield).

### Example 23. Preparation of (Pyrrolidin-2-Yl) Methyl 5-(4-Chlorobenzoyl)-1,4-Dimethyl-1H-Pyrrole-2-Acetate Hydrochloride

5-(4-Chlorobenzoyl)-1,4-dimethyl-1H-pyrrole-2-acetic acid (28.2 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride 19.2 (EDCI, g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (80% yield).

### Example 24. Preparation of (Pyrrolidin-2-Yl) Methyl 3-Chloro-4-(2-Propenyloxy) Benzeneacetate Hydrochloride

3-Chloro-4-(2-propenyloxy)benzeneacetic acid (20.8 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dDimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (87% yield).

### Example 25. Preparation of (Pyrrolidin-2-Yl) Methyl 2-(4-Isobutylphenyl) Propanoate Hydrochloride

2-(4-Isobutylphenyl) propanoic acid (20.6 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (91% yield).

### Example 26. Preparation of (Pyrrolidin-2-Yl) Methyl 2-Acetoxybenzoate Hydrochloride

2-Acetoxybenzoic acid (18.0 g) and N-Boc-L-prolinol (tert-butoxycarbonyl-2-pyrrolidinemethanol, 20.1 g) were put into 1 L round-bottom flask, acetone (200 added ml) was into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (89% yield).

### Example 27. Preparation of (Pyrrolidin-2-Yl) Methyl 5-(2,4-Difluorophenyl) Salicylate Hydrochloride

5-(2,4-Difluorophenyl) salicylic acid (25.0 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (78% yield).

### Example 28. Preparation of (Pyrrolidin-2-Yl) Methyl 5-(2,4-Difluorophenyl) Acetylsalicylate Hydrochloride

5-(2,4-Difluorophenyl) acetylsalicylic acid (29.2 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (71% yield).

### Example 29. Preparation of (Pyrrolidin-2-Yl) Methyl 2-(3-Benzoylphenyl) Propionate Hydrochloride

2-(3-Benzoylphenyl) propanoic acid (25.3 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, g), 4-19.2 dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (82% yield).

### Example 30. Preparation of (Pyrrolidin-2-Yl) Methyl 2-(3-Phenoxyphenyl) Propionate Hydrochloride

2-(3-phenoxylphenyl) propanoic acid (24.1 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (86% yield).

### Example 31. Preparation of (Pyrrolidin-2-Yl) Methyl (Z)-5-Fluoro-2-Methyl-1-[(4-Methylsulfinyl)Phenylmethylene]-1H-Indene-3-Acetate Hydrochloride

(Z)-5-fluoro-2-methyl-1-[(4-methylsulfinyl)phenylmethylene]-1H-indene-3-acetic acid (35.6 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (83% yield).

### Example 32. Preparation of (Pyrrolidin-2-Yl) Methyl 5-(4-Chlorobenzoyl)-1,4-Dimethyl-1H-Pyrrole-2-Acetate Hydrochloride

5-(4-Chlorobenzoyl)-1,4-dimethyl-1H-pyrrole-2-acetic acid (29.1 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (79% yield).

### Example 33. Preparation of (Pyrrolidin-2-Yl) Methyl α-Methyl-4-(2-Thienylcarbonyl)Benzeneacetate Hydrochloride

α-Methyl-4-(2-thienylcarbonyl)benzeneacetic acid (26.0 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (80% yield).

### Example 34. Preparation of (pyrrolidin-2-yl)methyl 6-chloro-α-methyl-9H-carbazole-2-acetate hydrochloride

6-Chloro-α-methyl-9H-carbazole-2-acetic acid (27.4 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (89% yield).

### Example 35. Preparation of (Pyrrolidin-2-Yl) Methyl 2-(4-Chlorophenyl)-α-Methyl-5-Benzoxazoleacetate Hydrochloride

2-(4-Chlorophenyl)-α-methyl-5-benzoxazoleacetic acid (30.2 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (78% yield).

### Example 36. Preparation of (Pyrrolidin-2-Yl) Methyl 2-13-Chloro-4-(3-Pyrrolin-1-Yl)Phenyl]Propanoate Hydrochloride

2-[3-Chloro-4-(3-pyrrolin-1-yl)phenyl]propionic acid (25.1 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (78% yield).

### Example 37. Preparation of (Pyrrolidin-2-Yl) Methyl α-Methyl-(4-Chlorobenzoyl)-5-Methoxy-2-Methylindole-3-Acetate Hydrochloride

α-Methyl-(4-chlorobenzoyl)-5-methoxy-2-methylindole-3-acetic acid (33.0 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (91% yield).

### Example 38. Preparation of (Pyrrolidin-2-Yl) Methyl 2-(5H-[1]-Benzopyrano[2,3-b]Pyridin-7-Yl) Propionate Hydrochloride

2-(5H-[1]-Benzopyrano[2,3-b]pyridin-7-yl) propionic acid (25.5 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (80% yield).

### Example 39. Preparation of (Pyrrolidin-2-Yl) Methyl α-Methyl-4-1 (2-Methyl-2-Propen-1-Yl) Amino]Benzeneacetate Hydrochloride

α-Methyl-4-[(2-methyl-2-propen-1-yl)amino]benzeneacetic acid (21.9 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (84% yield).

### Example 40. Preparation of (Pyrrolidin-2-Yl) Methyl 5~Benzoyl-α-Methyl-2-Thiopheneacetate Hydrochloride

5-Benzoyl-α-methyl-2-thiopheneacetic acid (26.0 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (77% yield).

### Example 41. Preparation of (pyrrolidin-2-yl)methyl 10,11~Dihydro-α-methyl-10-oxodibenzo]b,f]thiepin-2-acetate hydrochloride

10,11-Dihydro-α-methyl-10-oxodibenzo[b,/]thiepin-2-acetic acid (29.8 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (89% yield).

### Example 42. Preparation of (pyrrolidin-2-yl)methyl 2-(8-methyl-5-oxo-6H-benzo[b][1]benzoxepin-3-yl) propanoate hydrochloride

2-(8-Methyl-5-oxo-6H-benzo[b][1]benzoxepin-3-yl) propanoic acid (29.6 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride 19.2 (EDCI, g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (93% yield).

### Example 43. Preparation of (pyrrolidin-2-yl)methyl 2-14-[(2-Oxocyclopentyl)methyl]phenyl]propanoate hydrochloride

2-[4-[(2—Oxocyclopentyl)methyl]phenyl]propionic acid (24.6 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (90% yield).

### Example 44. Preparation of (Pyrrolidin-2-Yl) Methyl 4-(1,3-Dihydro-1—Oxo-2H-Isoindol-2-Yl)-α-Methylbenzeneacetate Hydrochloride

4-(1,3-Dihydro-1-oxo-2H-isoindol-2-yl)-α-methylbenzeneacetic acid (28.1 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (93% yield).

### Example 45. Preparation of (Pyrrolidin-2-Yl) Methyl 2-Chloro-2-(3-Chloro-4-Cyclohexylphenyl) Acetate Hydrochloride

2-Chloro-2-(3-chloro-4-cyclohexylphenyl) acetic acid (28.7 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (82% yield).

### Example 46. Preparation of (Pyrrolidin-2-Yl) Methyl 4,5-Diphenyl-2—Oxazolepropanoate Hydrochloride

4,5~Diphenyl-2-oxazolepropanoic acid (29.3 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (87% yield).

### Example 47. Preparation of (Pyrrolidin-2-Yl) Methyl 3-(4-Biphenylcarbonyl) Propanoate Hydrochloride

3-(4-Biphenylcarbonyl) propionic acid (25.4 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (81% yield).

### Example 48. Preparation of (Pyrrolidin-2-Yl) Methyl 3-15-(4-Chlorophenyl) Furan-2-yl]-3-Hydroxypropanoate Hydrochloride

3-[5-(4-Chlorophenyl) furan-2-yl]-3-hydroxypropanoic acid (26.7 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (85% yield).

### Example 49. Preparation of (Pyrrolidin-2-Yl) Methyl 6-Chloro-5-Cyclohexyl-1-Indancarboxylate Hydrochloride

6-Chloro-5-cyclohexyl-1-indancarboxylic acid (27.9 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (85% yield).

### Example 50. Preparation of (Pyrrolidin-2-Yl) Methyl 1-(4-Chlorobenzoyl)-5-Methoxy-2-Methylindole 3-Acetate Hydrochloride

1-(4-Chlorobenzoyl)-5-methoxy-2-methylindole 3-acetic acid (35.8 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (77% yield).

### Example 51. Preparation of (Pyrrolidin-2-Yl) Methyl 1-Methyl-5-(4-Methylbenzoyl)-1H-Pyrrole-2-Acetate Hydrochloride

1-Methyl-5-(4-methylbenzoyl)-1H-pyrrole-2-acetic acid (25.7 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (81% yield).

### Example 52. Preparation of (Pyrrolidin-2-Yl) Methyl 5-(4-Chlorobenzoyl)-1,4-Dimethyl-1H-Pyrrole-2-Acetate Hydrochloride

5-(4-Chlorobenzoyl)-1,4-dimethyl-1H-pyrrole-2-acetic acid (29.2 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (79% yield).

### Example 53. Preparation of (Pyrrolidin-2-Yl) Methyl 3-Chloro-4-(2-Propenyloxy)Benzeneacetate Hydrochloride

3-Chloro-4-(2-propenyloxy)benzeneacetic acid (22.7 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride 19.2 (EDCI, g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (79% yield).

### Example 54. Preparation of (Pyrrolidin-2-Yl) Methyl 3-2-(2,4-Dichlorophenoxy)Benzeneacetate Hydrochloride

3-2-(2,4-Dichlorophenoxy)benzeneacetic acid (29.7 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (79% yield).

### Example 55. Preparation of (Pyrrolidin-2-Yl) Methyl 4-Acetamidophenyl Salicylate Hydrochloride

4-Acetamidophenyl salicylic acid (27.1 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (79% yield).

### Example 56. Preparation of (Pyrrolidin-2-Yl) Methyl 2-[(2,3-Dimethylphenyl)Amino]Benzoate Hydrochloride

2-[(2,3-Dimethylphenyl)amino]benzoic acid (24.1 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (81% yield).

### Example 57. Preparation of (Pyrrolidin-2-Yl) Methyl 2-1 (2,3~Dimethylphenyl)Amino]Benzoate Hydrochloride

2-[(2,3-Dimethylphenyl)amino]benzoic acid (24.1 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (84% yield).

### Example 58. Preparation of (Pyrrolidin-2-Yl) Methyl 2-[[3-(Trifluoromethyl)Phenyl]Amino]-3-Pyridinecarboxylate Hydrochloride

2-[[3-(Trifluoromethyl)phenyl]amino]-3-pyridinecarboxylic acid (41.4 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (84% yield).

### Example 59. Preparation of (Pyrrolidin-2-Yl) Methyl 2-[(2,6-Dichloro-3-Methylphenyl)Amino]Benzoate Hydrochloride

2-[(2,6-Dichloro-3-methylphenyl)amino]benzoic acid (31.8 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (74% yield).

### Example 60. Preparation of (Pyrrolidin-2-Yl) Methyl 2-[[2-Methyl-3-(Trifluoromethyl)Phenyl]Amino]-3-Pyridinecarboxylate Hydrochloride

2-[2-Methyl-3-(trifluoromethyl)phenyl]amino]-3-pyridinecarboxylic acid (29.6 g) and N-Boc-L-prolinol (tert-butoxycarbonyl-2-pyrrolidinemethanol, 20.1 g) were put into 1 L round-bottom flask, acetone (200 ml) was added into the mixture. 1-Ethyl-(3-dimethylaminopropyl) carbodiimide hydrochloride (EDCI, 19.2 g), 4-dimethylaminopyridine (DMAP, 5 g), and 1-hydroxybenzotriazole (HOBt, 15 g) were added into the solution. The mixture was stirred for overnight at room temperature. The solution was evaporated to almost dryness. Ethyl acetate (500 ml) was added into the mixture. The solution is washed with water (2×200 ml), 20% citric acid (R0089, 50 g in 250 ml of water)×2, and water (3×300 ml). The solution is dried over sodium sulfate. Sodium sulfate was removed by filtration and washed with ethyl acetate (3×50 ml), the ethyl acetate solution was evaporated to dryness. 3N HCl in ethyl acetate (50 ml) was added, the mixture was stirred for 3 hours. The solid was collected and washed with ethyl acetate (5×50 ml) and dried in vacuum oven at 40° C. (74% yield).

### Example 61. The Skin Penetration Rates of HPP

The skin penetration rates of most of pro-drugs of NSAIDs have been reported in previous patents.

The penetration rates of (pyrrolidin-2-yl)methyl 4-[4-[3-(4-acetyl-3-hydroxy-2-propylphenoxy) propylsulfonyl]phenyl]-4-oxo-butanoate hydrochloride (Compound-1), 4-[4-[3-(4-acetyl-3-hydroxy-2-propylphenoxy) propylsulfonyl]phenyl]-4-oxo-butanoic acid (Parent drug-1), (pyrrolidin-2-yl)methyl 3-[[1-(4-chlorobenzyl)-4-methyl-6-(5-phenylpyridin-2-yl) methoxy]-4,5-dihydro-1H-thiopyrano[2,3,4-c,d]indol-2-yl]-2,2-dimethylpropanoate hydrochloride (Compound-2), 3-(1-(4-Chlorobenzyl)-3-t-butylthio-5-isopropylindol-2-yl)-2,2-dimethylpropanoic acid (parent drug-2), (Z)-2-(N,N-diethylaminoethyl) ethyl 2-(5-fluoro-2-methyl-1-(4-methylsulfinyl)benzylidene)-1H-inden-1-yl) acetate·AcOH (Compound-3), 2-(5-fluoro-2-methyl-1-(4-methylsulfinyl)benzylidene)-1H-inden-1-yl) acetic acid (parent drug-3), (pyrrolidin-2-yl)methyl 3-[3-[(E)-2-(7-chloroquinolin-2-yl) ethenyl]phenyl]-[3-(dimethylamino)-3-oxopropyl]sulfanylmethyl]sulfanylpropanoate·hydrochloride (Compound-4), 3-[3-[(E)-2-(7-chloroquinolin-2-yl) ethenyl]phenyl]-[3-(dimethylamino)-3-oxopropyl]sulfanylmethyl]sulfanylpropanoic acid (parent drug-4), (pyrrolidin-2-yl)methyl 2-cyclopentyl-2-[4-(quinolin-2-ylmethoxy)phenyl]acetate·hydrochloride (Compound-5), 2-cyclopentyl-2-[4-(quinolin-2-ylmethoxy)phenyl]acetic acid (parent drug-5), (RS)—N-[1-(1-benzothien-2-yl)ethyl]-N-(2-N,N-diethylaminoacetyloxy) urea·hydrochloride (Compound-6), (+)—N-hydroxy-N-(1-benzo[b]thien-2-ylethyl) urea (parent drug-6), 2-(N,N-diethylamino)ethyl 2-[1-[[(1R)-1-[3-[2-(7-chloroquinolin-2-yl) ethenyl]phenyl]-3-[2-(2-hydroxypropan-2-yl)phenyl]propyl]sulfanylmethyl]cyclopropyl]acetate·hydrochloride (Compound-7), 2-(N,N-diethylamino)ethyl 2-[1-[(1R)-1-[3-[2-(7-chloroquinolin-2-yl) ethenyl]phenyl]-3-[2-(2-hydroxypropan-2-yl)phenyl]propyl]sulfanylmethyl]cyclopropyl]acetic acid (parent drug-7), (pyrrolidin-2-yl)methyl 2-(2-(2,6-dichlorophenylamino)phenyl) acetate·hydrochloride (Compound-8) and 2-(2-(2,6-dichlorophenylamino)phenyl) acetic acid (parent drug-8) through rabbit skin were measured in vitro by using modified Franz cells, which were isolated from rabbit skin tissue (300-350 μm thick) of the back areas. The receiving fluid consisted of 10 ml of pure water are shown in table 1. The results suggest that the positive charge on the amino group has a very important role in the passage of the drug across the membrane and skin barrier.

TABLE 1
The Cumulative amounts of anti-inflammatory drugs in a period of 8 hours.

| | The cumulative amount of test compounds | | | |
|---|---|---|---|---|
| | 1 h | 2 h | 4 h | 8 h |
| Compound-1 | 0.50 mg | 1.02 mg | 2.18 mg | 4.48 mg |
| Parent drug-1 | 0.000 mg | 0.001 mg | 0.001 mg | 0.002 mg |
| Compound-2 | 0.43 mg | 0.93 mg | 2.03 mg | 4.19 mg |
| Parent drug-2 | 0.001 mg | 0.001 mg | 0.001 mg | 0.003 mg |
| Compound-3 | 0.51 mg | 1.05 mg | 2.13 mg | 4.31 mg |
| Parent drug-3 | 0.000 mg | 0.001 mg | 0.001 mg | 0.003 mg |
| Compound-4 | 0.46 mg | 1.01 mg | 2.09 mg | 4.29 mg |
| Parent drug-4 | 0.001 mg | 0.001 mg | 0.001 mg | 0.002 mg |
| Compound-5 | 0.52 mg | 1.03 mg | 2.10 mg | 4.19 mg |
| Parent drug-5 | 0.001 mg | 0.001 mg | 0.001 mg | 0.002 mg |
| Compound-6 | 0.39 mg | 0.86 mg | 1.91 mg | 3.89 mg |
| Parent drug-6 | 0.001 mg | 0.001 mg | 0.001 mg | 0.003 mg |
| Compound-7 | 0.36 mg | 0.78 mg | 1.77 mg | 3.87 mg |
| Parent drug-9 | 0.000 mg | 0.001 mg | 0.002 mg | 0.002 mg |
| Compound-8 | 0.48 mg | 0.99 mg | 2.03 mg | 4.09 mg |
| Parent drug-8 | 0.000 mg | 0.001 mg | 0.002 mg | 0.003 mg |

### Example 62. Antipyretic Activity

Randomized 84 Sprague Dawley rats into 7 groups (n=12), fever was induced by injecting 100 μg/kg LPS (dissolved in saline, 0.2 mg/mL) into abdomen of each rat, 2 hours later, 25% ethanol in water (group A, negative control group), Ibuprofen (oral, group B, 100 mg/kg, ibuprofen was suspended in 0.5% CMC-Na, final concentration of 20 mg/mL), 7% ibuprofen in 70% ethanol (topical, group C, 100 mg/kg), 7% of 2-(N,N-diethylamino)ethyl 2-(4-isobutylphenyl) propanoate·HCl in 25% ethanol (transdermal, group D, 20 mg/kg), 7% of (pyrrolidin-2-yl)methyl 2-(6-Methoxy-2-naphthyl) propanoate hydrochloride in 35% ethanol (transdermal, group E, 20 mg/kg), (pyrrolidin-2-yl)methyl 2-(2-(2,6-dichlorophenylamino)phenyl) acetate·hydrochloride (transdermal, group F, 20 mg/kg), and (pyrrolidin-2-yl)methyl 2-(2-Fluorobiphenyl-4-yl) propanoate hydrochloride (transdermal, group G, 20 mg/kg) were administered (topical and transdermal test articles were administered on the back of rats). The body temperature of rats was taken at 0 (2 hours after the challenge), 90 min, 180 min, and 270 min after the administration of the test compounds. The results are shown in Table 2.

TABLE 2
Antipyretic Activity of NSAIDs and related compounds.

| | | | | | T = 270 |
|---|---|---|---|---|---|
| Test article | | T = 0 min | T = 90 min | T = 180 min | min |
| A (Control | Mean | 38.32 | 38.34 | 38.22 | 37.92 |
| group) | SEM | 0.10 | 0.08 | 0.09 | 0.11 |
| B (oral, | Mean | 38.27 | 38.01 | 37.34 | 37.02 |
| 100 mg/kg) | SEM | 0.10 | 0.11 | 0.08 | 0.09 |
| C (topical, | Mean | 38.18 | 38.26 | 38.27 | 37.98 |
| 100 mg/kg) | SEM | 0.08 | 0.12 | 0.07 | 0.10 |
| D (transdermal, | Mean | 38.31 | 37.57 | 36.86 | 36.63 |
| 20 mg/kg) | SEM | 0.07 | 0.11 | 0.10 | 0.12 |
| E (transdermal, | Mean | 38.30 | 37.38 | 36.79 | 36.53 |
| 20 mg/kg) | SEM | 0.09 | 0.10 | 0.09 | 0.08 |
| F (transdermal, | Mean | 38.38 | 37.10 | 36.42 | 36.41 |
| 20 mg/kg) | SEM | 0.08 | 0.11 | 0.12 | 0.13 |
| G transdermal, | Mean | 38.30 | 37.37 | 36.78 | 36.65 |
| (20 mg/kg) | SEM | 0.10 | 0.09 | 0.10 | 0.12 |

The results showed that the prodrugs demonstrated strong antipyretic activity at 20 mg/kg dose and better than oral ibuprofen (100 mg/kg) and the topical ibuprofen (100 mg/kg) did not show any antipyretic activity.

### Example 63. Antiinflammatory Activity

Randomly grouped 50 Wistar Rats into 5 groups (n=10). Carrageenan solution was prepared as a 1% W/V solution in 0.9% saline, paw edema was induced by subcutaneous injection of 100 μL Carrageenan solution into right foot-pad, 2 hours later, 25% ethanol in water (G1, negative control group), Ibuprofen (oral, G2, 100 mg/kg, ibuprofen was suspended in 0.5% CMC-Na, final concentration of 20 mg/mL), 7% ibuprofen in 70% ethanol (topical, G3, 100 mg/kg), 7% of 2-(N,N-diethylamino)ethyl 2-(4-isobutylphenyl) propanoate·HCl in 25% ethanol (transdermal, G4, 20 mg/kg), and (pyrrolidin-2-yl)methyl 2-(2-(2,6-dichlorophenylamino)phenyl) acetate·hydrochloride (transdermal, G5, 20 mg/kg), were administered (topical and transdermal test articles were administered on the back of rats). The paw volume of rats were measured at 0 (before carrageenan challenge), 2 h (before test articles treatment) and 6 h (4 hours after the administration of the test compounds). The results are shown in Table 3.

TABLE 3
Paw Volume (mL).

| | G1 | | G2 | | G3 | | G4 | | G5 | |
|---|---|---|---|---|---|---|---|---|---|---|
| Times | Mean(mL) | SEM | Mean(mL) | SEM | Mean(mL) | SEM | Mean(mL) | SEM | Mean(mL) | SEM |
| 0 | 1.237 | 0.011 | 1.259 | 0.005 | 1.229 | 0.010 | 1.225 | 0.008 | 1.245 | 0.009 |
| 2 h | 1.754 | 0.073 | 1.757 | 0.026 | 1.765 | 0.019 | 1.697 | 0.039 | 1.719 | 0.042 |
| 6 h | 1.746 | 0.046 | 1.649 | 0.020 | 1.760 | 0.017 | 1.402 | 0.033 | 1.314 | 0.052 |

The results showed that the prodrugs demonstrated strong antiinflammatory activity at 20 mg/kg dose and better than oral ibuprofen (100 mg/kg) and the topical ibuprofen (100 mg/kg) did not show any anti-inflammatory activity.

### Example 64. Efficacy of 2-(Diethylamino)Ethyl 2-(4-Isobutylphenyl) Propanoate·Hydrochloride and 2-(Diethylamino)Ethyl 2-Acetoxybenzoate Hydrochloride in Treatment of Signs, Symptoms, and/or Complications of H1N1 Virus Infection

A total of one hundred ten (110) mice were used in this study. Four (4) groups of ten (10) were used for virus titration study and seven (7) groups of ten (10) were used for efficacy in the relief of signs and symptoms of virus infection of test article study. All mice were challenged with a lethal dose of mouse adapted Influenza A PR/8/34 (H1N1) by intranasal instillation on Study Day 0. A single dose of 10×TCID₅₀/mouse, 1×TCID₅₀/mouse, 0.1×TCID₅₀/mouse, or 0.01×TCID₅₀/mouse Virus was administered via nasal mucosa by pipette in virus titration study. In efficacy study, at day 0, mice were anesthetized with isoflurane and held in a dorsal recumbent position for administration of 0.1×TCID₅₀/mouse virus by internasal instillation, from day 2 post challenge, mice were received a single dose of different concentrations of test article or vehicle control once a day up to 15th day with dose initiation on day 2 post challenge. Group 1 served as an infected control group and was treated with vehicle (25% ethanol). 7% 2-(Diethylamino)ethyl 2-acetoxybenzoate hydrochloride in 15% ethanol (low dose: 25.2 mg/kg, group 2, mid dose: 50.4 mg/kg, group 3, and high dose: 100.8 mg/kg, group 4), and 6.25% 2-(diethylamino)ethyl 2-(4-isobutylphenyl) propanoate hydrochloride in 25% ethanol (low dose: 22.5 mg/kg, group 5, mid dose: 45 mg/kg, group 6, and high dose: 90 mg/kg, group 7) and vehicle (25% ethanol) were given by transdermal on the back of animal in an area of 2 cm×5 cm, 1.5 cm×3.5 cm, or 1 cm×2.5 cm for high, middle or low dose. Mice were observed following viral challenge for clinical signs of illness including body weights, health score, morbidity and mortality.

### Mortality

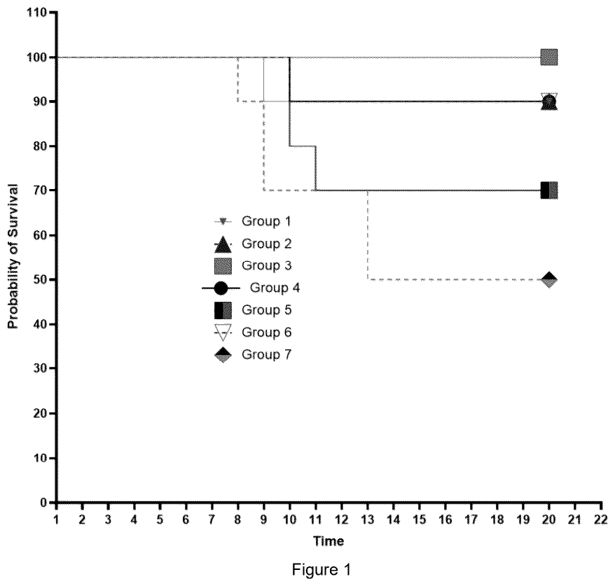

For efficacy study the survival of Balb/c mice challenged with a lethal dose of mouse-adapted Influenza A PR/8/34 (H1N1) is presented as a Kaplan-Meier Survival Curves in FIG. 1. 100.8 mg/kg (high dose, group 4) of 2-(diethylamino)ethyl 2-acetoxybenzoate hydrochloride provided the greatest amount of protection with 100% of the animals surviving. 90% of survival were obtained with both of 25.2 mg/kg (low dose, group 2) and 50.4 mg/kg (mid dose, group 3) of 2-(diethylamino)ethyl 2-acetoxybenzoate hydrochloride, 90% of survival was obtained with 22.5 mg/kg (low dose, group 5) and 90 mg/kg (high dose, group 7) of 2-(diethylamino)ethyl 2-(4-isobutylphenyl) propanoate·hydrochloride, 70% of survival was obtained with 45 mg/kg (mid dose, group 6, 3 died) 2-(diethylamino)ethyl 2-(4-isobutylphenyl) propanoate hydrochloride, and 50% of survival was obtained with vehicle (25% ethanol, group 1, 5 died) as shown in FIG. 1 and Table 4.

TABLE 4
Effect of drug on survival.

| Group No. | Group 1 | Group 2 | Group 3 | Group 4 | Group 5 | Group 6 | Group 7 |
|---|---|---|---|---|---|---|---|
| Animals | 5/10 | 1/10 | 1/10 | 0/10 | 1/10 | 3/10 | 1/10 |
| died/total | | | | | | | |

### Lung Infection Characterization

Lung lesions and peribronchiolitis of all deceased and surviving animals were characterized (Table 5).

TABLE 5
Lung lesions chraracterized in animals.

| Group No. | Group 1 | Group 2 | Group 3 | Group 4 | Group 5 | Group 6 | Group 7 |
|---|---|---|---|---|---|---|---|
| Animals with lung | 5/10 | 5/10 | 1/10 | 1/10 | 0/10 | 0/10 | 0/10 |
| lesion/total | | | | | | | |

### Conclusion

The purpose of this study was to evaluate relief of the signs, symptoms and/or complications of virus infection of 2-(diethylamino)ethyl 2-acetoxybenzoate hydrochloride and 2-(diethylamino)ethyl 2-(4-isobutylphenyl) propanoate hydrochloride by transdermal administration on the back of animal (in an area of 2 cm×5 cm, 1.5 cm×3.5 cm, or 1 cm×2.5 cm for high, middle or low dose) in a mouse model of influenza A virus infection. Results demonstrated that administration of 2-(diethylamino)ethyl 2-acetoxybenzoate hydrochloride and 2-(diethylamino)ethyl 2-(4-isobutylphenyl) propanoate hydrochloride protected mice from death and lung damages, delayed and reduced the severity of clinical signs of illness.

### Example 65. Efficacy of (Pyrrolidin-2-Yl) Methyl 2-(2-(2,6-Dichlorophenylamino)Phenyl) Acetate·Hydrochloride and (Pyrrolidin-2-Yl) Methyl 2-(6-Methoxy-2-Naphthyl) Propanoate Hydrochloride in Treatment of Signs, Symptoms, and/or Complications of H1N1 Virus Infection

A total of seventy mice (7 groups, 10/group) were used in this study. At day 0, mice were anesthetized with isoflurane and held in a dorsal recumbent position for administration of 0.1×TCID₅₀/mouse virus (new batch) by internasal instillation, from day 2 post challenge, mice were received a single dose of different concentrations of test article or vehicle control once a day up to 15th day with dose initiation on day 2 post challenge. Group 1 served as an infected control group and was treated with vehicle (35% ethanol). 7% of (pyrrolidin-2-yl)methyl 2~ (6-methoxy-2-naphthyl) propanoate hydrochloride in 35% ethanol (low dose: 25.1 mg/kg, group 2, mid dose: 50.2 mg/kg, group 3, and high dose: 100.3 mg/kg, group 4) and 7% of (pyrrolidin-2-yl)methyl 2-(2-(2,6-dichlorophenylamino)phenyl) acetate·hydrochloride in 35% ethanol (low dose: 24.6 mg/kg, group 5, mid dose: 49.2 mg/kg, group 6, and high dose: 98.4 mg/kg, group 7) and vehicle (35% ethanol) were given by transdermal on the back of animal in an area of 2 cm×5 cm, 1.5 cm×3.5 cm, or 1 cm×2.5 cm for high, middle or low dose. Mice were observed following viral challenge for clinical signs of illness including body weights, health score, morbidity and mortality.

### Mortality

10% of survival was obtained with vehicle (35% ethanol, group 1, 9 died), 20% of survival was obtained with 25.1 mg/kg (group 2, 8 died) and 50.2 mg/kg (group 3, 8 died) of (pyrrolidin-2-yl)methyl 2-(6-methoxy-2-naphthyl) propanoate hydrochloride, 40% of survival was obtained with 100.3 mg/kg (group 4, 6 died) of (pyrrolidin-2-yl)methyl 2-(6-methoxy-2-naphthyl) propanoate hydrochloride, 70% of survival were obtained with 24.6 mg/kg (group 5, 3 died) of (pyrrolidin-2-yl)methyl 2-(2-(2,6-dichlorophenylamino)phenyl) acetate·hydrochloride, 60% of survival was obtained with 49.2 mg/kg (group 6, 4 died) of (pyrrolidin-2-yl)methyl 2-(2-(2,6-dichlorophenylamino)phenyl) acetate·hydrochloride, 50% of survival was obtained with 98.4 mg/kg (group 7, 5 died) (pyrrolidin-2-yl)methyl 2-(2-(2,6-dichlorophenylamino)phenyl) acetate hydrochloride as shown in Table 6.

TABLE 6
Effect of drug on survival.

| Group No. | Group 1 | Group 2 | Group 3 | Group 4 | Group 5 | Group 6 | Group 7 |
|---|---|---|---|---|---|---|---|
| Animals | 9/10 | 8/10 | 8/10 | 6/10 | 3/10 | 4/10 | 5/10 |
| died/total | | | | | | | |

### Lung Infection Characterization

Lung lesions and peribronchiolitis of all deceased and surviving animals were characterized (Table 7).

TABLE 7
Lung lesions chraracterized in animals.

| Group No. | Group 1 | Group 2 | Group 3 | Group 4 | Group 5 | Group 6 | Group 7 |
|---|---|---|---|---|---|---|---|
| Animals with lung | 9/10 | 8/10 | 6/10 | 4/10 | 3/10 | 3/10 | 3/10 |
| lesion/total | | | | | | | |

### Conclusion

The purpose of this study was to evaluate relief of the signs, symptoms and/or complications of virus infection of (pyrrolidin-2-yl)methyl 2-(6-methoxy-2-naphthyl) propanoate hydrochloride and (pyrrolidin-2-yl)methyl 2-(2-(2,6-dichlorophenylamino)phenyl) acetate·hydrochloride by transdermal administration on the back of animal (in an area of 2 cm×5 cm, 1.5 cm×3.5 cm, or 1 cm×2.5 cm for high, middle or low dose) in a mouse model of influenza A virus infection. Results demonstrated that administration of (pyrrolidin-2-yl)methyl 2-(6-methoxy-2-naphthyl) propanoate hydrochloride and (pyrrolidin-2-yl)methyl 2-(2-(2,6-dichlorophenylamino)phenyl) acetate·hydrochloride protected mice from death and lung damages, delayed and reduced the severity of clinical signs of illness.

### Example 66. Efficacy of (pyrrolidin-2-yl)methyl 2-(2-fluorobiphenyl-4-yl) propanoate hydrochloride and (Z)-2-(N,N-diethylaminoethyl) ethyl 2-(5-fluoro-2-methyl-1-(4-methylsulfinyl)benzylidene)-1H-inden-1-yl) acetate·ACOH in treatment of signs, symptoms, and/or complications of H1N1 virus infection

A total of seventy mice (7 groups, 10/group) were used in this study. At day 0, mice were anesthetized with isoflurane and held in a dorsal recumbent position for administration of 0.1×TCID₅₀/mouse virus (new batch) by internasal instillation, from day 2 post challenge, mice were received a single dose of different concentrations of test article or vehicle control once a day up to 15th day with dose initiation on day 2 post challenge. Group 1 served as an infected control group and was treated with vehicle (35% ethanol). 7% of (pyrrolidin-2-yl)methyl 2-(2-fluorobiphenyl-4-yl) propanoate hydrochloride in 35% ethanol (low dose: 25.0 mg/kg, group 2, mid dose: 50.0 mg/kg, group 3, and high dose: 100.0 mg/kg, group 4) and 7% of (Z)-2-(N,N-diethylaminoethyl) ethyl 2-(5-fluoro-2-methyl-1-(4-methylsulfinyl)benzylidene)-1H-inden-1-yl) acetate·ACOH in 35% ethanol (low dose: 25.5 mg/kg, group 5, mid dose: 50.9 mg/kg, group 6, and high dose: 101.9 mg/kg, group 7) and vehicle (35% ethanol) were given by transdermal on the back of animal in an area of 2 cm×5 cm, 1.5 cm×3.5 cm, or 1 cm×2.5 cm for high, middle or low dose. Mice were observed following viral challenge for clinical signs of illness including body weights, health score, morbidity and mortality.

### Mortality

20% of survival was obtained with vehicle (group 1, 8 died), 40% of survival was obtained with 25.0 mg/kg (group 2, 6 died) of (pyrrolidin-2-yl)methyl 2-(2-fluorobiphenyl-4-yl) propanoate hydrochloride, 50% of survival was obtained with 50.0 mg/kg (group 3, 5 died) of (pyrrolidin-2-yl)methyl 2-(2-fluorobiphenyl-4-yl) propanoate hydrochloride, 60% of survival was obtained with 100.0 mg/kg (group 4, 4 died) of (pyrrolidin-2-yl)methyl 2-(2-fluorobiphenyl-4-yl) propanoate hydrochloride, 60% of survival were obtained with 25.5 mg/kg (group 5, 4 died) of (Z)-2-(N,N-diethylaminoethyl) ethyl 2-(5-fluoro-2-methyl-1-(4-methylsulfinyl)benzylidene)-1H-inden-1-yl) acetate·ACOH, 70% of survival was obtained with 50.9 mg/kg (group 6, 3 died) of (Z)-2-(N,N-diethylaminoethyl) ethyl 2-(5-fluoro-2-methyl-1-(4-methylsulfinyl)benzylidene)-1H-inden-1-yl) acetate·ACOH, 50% of survival was obtained with 101.9 mg/kg (group 7, 5 died) (Z)-2-(N,N-diethylaminoethyl) ethyl 2-(5-fluoro-2-methyl-1-(4-methylsulfinyl)benzylidene)-1H-inden-1-yl) acetate·ACOH as shown in Table 8.

TABLE 8
Effect of drug on survival.

| Group No. | Group 1 | Group 2 | Group 3 | Group 4 | Group 5 | Group 6 | Group 7 |
|---|---|---|---|---|---|---|---|
| Animals | 8/10 | 6/10 | 5/10 | 4/10 | 4/10 | 3/10 | 5/10 |
| died/total | | | | | | | |

### Lung Infection Characterization

Lung lesions and peribronchiolitis of all deceased and surviving animals were characterized (Table 9).

TABLE 9
Lung lesions chraracterized in animals.

| Group No. | Group 1 | Group 2 | Group 3 | Group 4 | Group 5 | Group 6 | Group 7 |
|---|---|---|---|---|---|---|---|
| Animals with lung | 9/10 | 7/10 | 5/10 | 4/10 | 2/10 | 2/10 | 3/10 |
| lesion/total | | | | | | | |

### Conclusion

The purpose of this study was to evaluate relief of the signs, symptoms and/or complications of virus infection of (pyrrolidin-2-yl)methyl 2-(2-fluorobiphenyl-4-yl) propanoate hydrochloride and (Z)-2-(N,N-diethylaminoethyl) ethyl 2-(5-fluoro-2-methyl-1-(4-methylsulfinyl)benzylidene)-1H-inden-1-yl) acetate·ACOH by transdermal administration on the back of animal (in an area of 2 cm×5 cm, 1.5 cm×3.5 cm, or 1 cm×2.5 cm for high, middle or low dose) in a mouse model of influenza A virus infection. Results demonstrated that administration of (pyrrolidin-2-yl)methyl 2-(2-fluorobiphenyl-4-yl) propanoate hydrochloride and (Z)-2-(N,N-diethylaminoethyl) ethyl 2-(5-fluoro-2-methyl-1-(4-methylsulfinyl)benzylidene)-1H-inden-1-yl) acetate·ACOH protected mice from death and lung damages, delayed and reduced the severity of clinical signs of illness.

### Example 67. Efficacy of (Pyrrolidin-2-Yl) Methyl 2-1 (2,3-dimethylphenyl)Amino]Benzoate Hydrochloride and (Pyrrolidin-2-Yl) Methyl α-Methyl-4-(2-Thienylcarbonyl)Benzeneacetate Hydrochloride in Treatment of Signs, Symptoms, and/or Complications of H1N1 Virus Infection

A total of seventy mice (7 groups, 10/group) were used in this study. At day 0, mice were anesthetized with isoflurane and held in a dorsal recumbent position for administration of 0.1×TCID₅₀/mouse virus (new batch) by internasal instillation, from day 2 post challenge, mice were received a single dose of different concentrations of test article or vehicle control once a day up to 15th day with dose initiation on day 2 post challenge. Group 1 served as an infected control group and was treated with vehicle (35% ethanol). 7% of (pyrrolidin-2-yl)methyl 2-[(2,3-dimethylphenyl)amino]benzoate hydrochloride in 35% ethanol (low dose: 25.0 mg/kg, group 2, mid dose: 50.1 mg/kg, group 3, and high dose: 100.1 mg/kg, group 4) and 7% of (pyrrolidin-2-yl)methyl α-methyl-4-(2-thienylcarbonyl)benzeneacetate hydrochloride in 35% ethanol (low dose: 24.9 mg/kg, group 5, mid dose: 49.8 mg/kg, group 6, and high dose: 99.6 mg/kg, group 7) and vehicle (35% ethanol) were given by transdermal on the back of animal in an area of 2 cm×5 cm, 1.5 cm×3.5 cm, or 1 cm×2.5 cm for high, middle or low dose. Mice were observed following viral challenge for clinical signs of illness including body weights, health score, morbidity and mortality.

### Mortality

10% of survival was obtained with vehicle (group 1, 9 died), 30% of survival was obtained with 25.0 mg/kg (group 2, 7 died) of (pyrrolidin-2-yl)methyl 2-[(2,3~ dimethylphenyl)amino]benzoate hydrochloride, 50% of survival was obtained with 50.1 mg/kg (group 3, 5 died) of (pyrrolidin-2-yl)methyl 2-[(2,3-dimethylphenyl)amino]benzoate hydrochloride, 50% of survival was obtained with 100.1 mg/kg (group 4, 5 died) of (pyrrolidin-2-yl)methyl 2-[(2,3-dimethylphenyl)amino]benzoate hydrochloride, 60% of survival were obtained with 24.9 mg/kg (group 5, 4 died) of (pyrrolidin-2-yl)methyl α-methyl-4-(2-thienylcarbonyl)benzeneacetate hydrochloride, 50% of survival was obtained with 49.8 mg/kg (group 6, 5 died) of (pyrrolidin-2-yl)methyl α-methyl-4-(2-thienylcarbonyl)benzeneacetate hydrochloride, 40% of survival was obtained with 99.6 mg/kg (group 7, 6 died) (pyrrolidin-2-yl)methyl α-methyl-4-(2-thienylcarbonyl)benzeneacetate hydrochloride as shown in Table 10.

TABLE 10
Effect of drug on survival.

| Group No. | Group 1 | Group 2 | Group 3 | Group 4 | Group 5 | Group 6 | Group 7 |
|---|---|---|---|---|---|---|---|
| Animals | 9/10 | 7/10 | 5/10 | 5/10 | 4/10 | 5/10 | 6/10 |
| died/total | | | | | | | |

### Lung Infection Characterization

Lung lesions and peribronchiolitis of all deceased and surviving animals were characterized (Table 11).

TABLE 11
Lung lesions chraracterized in animals.

| Group No. | Group 1 | Group 2 | Group 3 | Group 4 | Group 5 | Group 6 | Group 7 |
|---|---|---|---|---|---|---|---|
| Animals with lung | 9/10 | 7/10 | 5/10 | 4/10 | 3/10 | 3/10 | 3/10 |
| lesion/total | | | | | | | |

### Conclusion

The purpose of this study was to evaluate relief of the signs, symptoms and/or complications of virus infection of (pyrrolidin-2-yl)methyl 2-[(2,3~ dimethylphenyl)amino]benzoate hydrochloride and (pyrrolidin-2-yl)methyl α-methyl-4-(2-thienylcarbonyl)benzeneacetate hydrochloride by transdermal administration on the back of animal (in an area of 2 cm×5 cm, 1.5 cm×3.5 cm, or 1 cm×2.5 cm for high, middle or low doses) in a mouse model of influenza A virus infection. Results demonstrated that administration of (pyrrolidin-2-yl)methyl 2-[(2,3-dimethylphenyl)amino]benzoate hydrochloride and (pyrrolidin-2-yl)methyl α-methyl-4-(2-thienylcarbonyl)benzeneacetate hydrochloride protected mice from death and lung damages, delayed and reduced the severity of clinical signs of illness.

### Example 68. Efficacy of (Pyrrolidin-2-Yl) Methyl 5-(2,4-Difluorophenyl) Salicylate Hydrochloride and (Pyrrolidin-2-Yl) Methyl 5-(2,4-Difluorophenyl) Acetylsalicylate Hydrochloride in Treatment of Signs, Symptoms, and/or Complications of H1N1 Virus Infection

A total of seventy mice (7 groups, 10/group) were used in this study. At day 0, mice were anesthetized with isoflurane and held in a dorsal recumbent position for administration of 0.1×TCID₅₀/mouse virus (new batch) by internasal instillation, from day 2 post challenge, mice were received a single dose of different concentrations of test article or vehicle control once a day up to 15th day with dose initiation on day 2 post challenge. Group 1 served as an infected control group and was treated with vehicle (35% ethanol). 7% (pyrrolidin-2-yl)methyl 5-(2,4-difluorophenyl) salicylate hydrochloride in 35% ethanol (low dose: 25.0 mg/kg, group 2, mid dose: 49.9 mg/kg, group 3, and high dose: 99.8 mg/kg, group 4) and 7% (pyrrolidin-2-yl)methyl 5-(2,4-difluorophenyl) acetylsalicylate hydrochloride in 35% ethanol (low dose: 24.7 mg/kg, group 5, mid dose: 49.4 mg/kg, group 6, and high dose: 98.7 mg/kg, group 7) and vehicle (35% ethanol) were given by transdermal on the back of animal in an area of 2 cm×5 cm, 1.5 cm×3.5 cm, or 1 cm×2.5 cm for high, middle or low dose. Mice were observed following viral challenge for clinical signs of illness including body weights, health score, morbidity and mortality.

### Mortality

20% of survival was obtained with vehicle (group 1, 8 died), 30% of survival was obtained with 25.0 mg/kg (group 2, 7 died) of (pyrrolidin-2-yl)methyl 5-(2,4-difluorophenyl) salicylate hydrochloride, 50% of survival was obtained with 49.9 mg/kg (group 3, 5 died) of (pyrrolidin-2-yl)methyl 5-(2,4-difluorophenyl) salicylate hydrochloride, 60% of survival was obtained with 99.8 mg/kg (group 4, 4 died) of (pyrrolidin-2-yl)methyl 5-(2,4-difluorophenyl) salicylate hydrochloride, 40% of survival were obtained with 24.7 mg/kg (group 5, 6 died) of (pyrrolidin-2-yl)methyl 5-(2,4-difluorophenyl) acetylsalicylate hydrochloride, 60% of survival was obtained with 49.4 mg/kg (group 6, 4 died) of (pyrrolidin-2-yl)methyl 5-(2,4-difluorophenyl) acetylsalicylate hydrochloride, 70% of survival was obtained with 98.7 mg/kg (group 7, 3 died) (Z)-2-(N,N-diethylaminoethyl) ethyl 2-(5-fluoro-2-methyl-1-(4-methylsulfinyl)benzylidene)-1H-inden-1-yl) acetate·ACOH (pyrrolidin-2-yl)methyl 5-(2,4-difluorophenyl) acetylsalicylate hydrochloride as shown in Table 12.

TABLE 12
Effect of drug on survival.

| Group No. | Group 1 | Group 2 | Group 3 | Group 4 | Group 5 | Group 6 | Group 7 |
|---|---|---|---|---|---|---|---|
| Animals | 8/10 | 7/10 | 5/10 | 4/10 | 6/10 | 4/10 | 3/10 |
| died/total | | | | | | | |

### Lung Infection Characterization

Lung lesions and peribronchiolitis of all deceased and surviving animals were characterized (Table 13).

TABLE 13
Lung lesions chraracterized in animals.

| Group No. | Group 1 | Group 2 | Group 3 | Group 4 | Group 5 | Group 6 | Group 7 |
|---|---|---|---|---|---|---|---|
| Animals with lung | 8/10 | 7/10 | 5/10 | 4/10 | 4/10 | 2/10 | 1/10 |
| lesion/total | | | | | | | |

### Conclusion

The purpose of this study was to evaluate relief of the signs, symptoms and/or complications of virus infection of (pyrrolidin-2-yl)methyl 5-(2,4-difluorophenyl) salicylate hydrochloride and (pyrrolidin-2-yl)methyl 5-(2,4-difluorophenyl) acetylsalicylate hydrochloride by transdermal administration on the back of animal (in an area of 2 cm×5 cm, 1.5 cm×3.5 cm, or 1 cm×2.5 cm for high, middle or low doses) in a mouse model of influenza A virus infection. Results demonstrated that administration of (pyrrolidin-2-yl)methyl 5-(2,4-difluorophenyl) salicylate hydrochloride and (pyrrolidin-2-yl)methyl 5-(2,4-difluorophenyl) acetylsalicylate hydrochloride protected mice from death and lung damages, delayed and reduced the severity of clinical signs of illness.

### Example 69. Efficacy of 2-(Diethylamino)Ethyl 2-Acetoxybenzoate Hydrochloride and (Pyrrolidin-2-Yl) Methyl 2-(2-(2,6-Dichlorophenylamino)Phenyl) Acetate Hydrochloride in Treatment of Signs, Symptoms, and/or Complications of COVID-19 Infection

A total of one hundred ten (110) hACE2 mice were used in this study. Four (4) groups of ten (10) were used for virus titration study and seven (7) groups of ten (10) were used for efficacy in the relief of signs and symptoms of COVID-19 virus infection of test article study. All mice were challenged with a lethal dose of mouse adapted Covid-19 by intranasal instillation on Study Day 0. A single dose of 5.5×10⁴. PFU/mouse, 5.5×10³. PFU/mouse, 5.5×10². PFU/mouse, or 5.5×10 PFU/mouse COVID-19 was administered via nasal mucosa by pipette in virus titration study. In efficacy study, at day 0, mice were anesthetized with isoflurane and held in a dorsal recumbent position for administration of 3×10 PFU/mouse by internasal instillation, from day 1 post challenge, mice were received a single dose of different concentrations of test article or vehicle control once a day up to 15th day with dose initiation on day 1 post challenge. Group 1 served as an infected control group and was treated with vehicle (25% ethanol). 7% 2-(diethylamino)ethyl 2-acetoxybenzoate hydrochloride in 25% ethanol (low dose: 25.2 mg/kg, group 2, mid dose: 50.4 mg/kg, group 3, and high dose: 100.8 mg/kg, group 4), and 6.25% (pyrrolidin-2-yl)methyl 2-(2-(2,6-dichlorophenylamino)phenyl) acetate hydrochloride in 25% ethanol (low dose: 22.5 mg/kg, group 5, mid dose: 45 mg/kg, group 6, and high dose: 90 mg/kg, group 7) and vehicle (25% ethanol) were given by transdermal on the back of animal in an area of 2 cm×5 cm, 1.5 cm×3.5 cm, or 1 cm×2.5 cm for high, middle or low dose. Mice were observed following viral challenge for clinical signs of illness including body weights, health score, morbidity and mortality.

### Mortality

For efficacy study the survival of hACE2 mice challenged with a lethal dose of mouse-adapted COVID-19 is presented in Table 10. 100.8 mg/kg (high dose, group 4, 1 died) of 2-(diethylamino)ethyl 2-acetoxybenzoate hydrochloride provided the greatest amount of protection with 90% of the animals surviving, 40% of survival were obtained with 25.2 mg/kg (low dose, group 2, 6 died) and 70% of survival were obtained with 50.4 mg/kg (mid dose, group 3, 3 died) of 2-(diethylamino)ethyl 2-acetoxybenzoate hydrochloride, 70% of survival was obtained with 22.5 mg/kg (low dose, group 5, 3 died) of (pyrrolidin-2-yl)methyl 2-(2-(2,6-dichlorophenylamino)phenyl) acetate hydrochloride, 60% of survival was obtained with 45 mg/kg (mid dose, group 6, 4 died) of (pyrrolidin-2-yl)methyl 2-(2-(2,6-dichlorophenylamino)phenyl) acetate hydrochloride, 40% of survival was obtained with 90 mg/kg (high dose, group 7, 6 died) (pyrrolidin-2-yl)methyl 2-(2-(2,6-dichlorophenylamino)phenyl) acetate hydrochloride, and 10% of survival was obtained with vehicle (25% ethanol, group 1, 9 died) as shown in Table 14.

TABLE 14
Effect of drug on survival.

| Group No. | Group 1 | Group 2 | Group 3 | Group 4 | Group 5 | Group 6 | Group 7 |
|---|---|---|---|---|---|---|---|
| Animals | 9/10 | 6/10 | 3/10 | 1/10 | 3/10 | 4/10 | 6/10 |
| died/total | | | | | | | |

### Lung Infection Characterization

Lung lesions and peribronchiolitis of all deceased and surviving animals were characterized (Table 15).

TABLE 15
Lung lesions chraracterized in animals.

| Group No. | Group 1 | Group 2 | Group 3 | Group 4 | Group 5 | Group 6 | Group 7 |
|---|---|---|---|---|---|---|---|
| Animals with lung | 10/10 | 6/10 | 4/10 | 1/10 | 4/10 | 6/10 | 5/10 |
| lesion/total | | | | | | | |

### Conclusion

The purpose of this study was to evaluate relief of the signs, symptoms and/or complications of COVID-19 infection of 2-(diethylamino)ethyl 2-acetoxybenzoate hydrochloride and (pyrrolidin-2-yl)methyl 2-(2-(2,6-dichlorophenylamino)phenyl) acetate hydrochloride by transdermal administration on the back of animal (in an area of 2 cm×5 cm, 1.5 cm×3.5 cm, or 1 cm×2.5 cm for high, middle or low dose) in a mouse model of COVID-19 infection. Results demonstrated that administration of 2-(diethylamino)ethyl 2-acetoxybenzoate hydrochloride and (pyrrolidin-2-yl)methyl 2-(2-(2,6-dichlorophenylamino)phenyl) acetate hydrochloride protected mice from death and lung damages, delayed and reduced the severity of clinical signs of illness.

### Example 70. Efficacy of 2-(Diethylamino)Ethyl 2-(4-Isobutylphenyl) Propanoate Hydrochloride and (Pyrrolidin-2-Yl) Methyl 2-(6-Methoxy-2-Naphthyl) Propanoate Hydrochloride in Treatment of Signs, Symptoms, and/or Complications of COVID-19 Infection

A total of seventy hACE2 mice (7 groups, 10/group) were used in this study. At day 0, mice were anesthetized with isoflurane and held in a dorsal recumbent position for administration of 3×10 PFU/mouse COVID-19 by internasal instillation, from day 1 post challenge, mice were received a single dose of different concentrations of test article or vehicle control once a day up to 15th day with dose initiation on day 1 post challenge. Group 1 served as an infected control group and was treated with vehicle (35% ethanol). 7% of (pyrrolidin-2-yl)methyl 2-(6-methoxy-2-naphthyl) propanoate hydrochloride in 35% ethanol (low dose: 25.1 mg/kg, group 2, mid dose: 50.2 mg/kg, group 3, and high dose: 100.3 mg/kg, group 4) and 7% of 2-(diethylamino)ethyl 2-(4-isobutylphenyl) propanoate hydrochloride in 35% ethanol (low dose: 24.6 mg/kg, group 5, mid dose: 49.2 mg/kg, group 6, and high dose: 98.4 mg/kg, group 7) and vehicle (35% ethanol) were given by transdermal on the back of animal in an area of 2 cm×5 cm, 1.5 cm×3.5 cm, or 1 cm×2.5 cm for high, middle or low dose. Mice were observed following viral challenge for clinical signs of illness including body weights, health score, morbidity and mortality.

### Mortality

10% of survival was obtained with vehicle (35% ethanol, group 1, 9 died), 30% of survival was obtained with 25.1 mg/kg (group 2, 7 died) of (pyrrolidin-2-yl)methyl 2-(6-Methoxy-2-naphthyl) propanoate hydrochloride, 50% of survival was obtained with 50.2 mg/kg (group 3, 5 died) of (pyrrolidin-2-yl)methyl 2-(6-Methoxy-2-naphthyl) propanoate hydrochloride, 50% of survival was obtained with 100.3 mg/kg (group 4, 5 died) of (pyrrolidin-2-yl)methyl 2-(6-methoxy-2-naphthyl) propanoate hydrochloride, 60% of survival were obtained with 24.6 mg/kg (group 5, 4 died) of 2-(diethylamino)ethyl 2-(4-isobutylphenyl) propanoate hydrochloride, 50% of survival was obtained with 49.2 mg/kg (group 6, 5 died) of 2-(diethylamino)ethyl 2-(4-isobutylphenyl) propanoate hydrochloride, 50% of survival was obtained with 98.4 mg/kg (group 7, 2 died due to the signs and symptoms of ARDS) 2-(diethylamino)ethyl 2-(4-isobutylphenyl) propanoate hydrochloride as shown in Table 16.

TABLE 16
Effect of drug on survival.

| Group No. | Group 1 | Group 2 | Group 3 | Group 4 | Group 5 | Group 6 | Group 7 |
|---|---|---|---|---|---|---|---|
| Animals | 9/10 | 7/10 | 5/10 | 5/10 | 4/10 | 5/10 | 5/10 |
| died/total | | | | | | | |

### Lung Infection Characterization

Lung lesions and peribronchiolitis of all deceased and surviving animals were characterized (Table 17).

TABLE 17
Lung lesions characterized in animals.

| Group No. | Group 1 | Group 2 | Group 3 | Group 4 | Group 5 | Group 6 | Group 7 |
|---|---|---|---|---|---|---|---|
| Animals with lung | 10/10 | 7/10 | 5/10 | 4/10 | 4/10 | 4/10 | 4/10 |
| lesion/total | | | | | | | |

### Conclusion

The purpose of this study was to evaluate relief of the signs, symptoms and/or complications of COVID-19 infection of (pyrrolidin-2-yl)methyl 2-(6-methoxy-2-naphthyl) propanoate hydrochloride and 2-(diethylamino)ethyl 2-(4-isobutylphenyl) propanoate hydrochloride by transdermal administration on the back of animal (in an area of 2 cm×5 cm, 1.5 cm×3.5 cm, or 1 cm×2.5 cm for high, middle or low dose) in a mouse model of COVID-19 infection. Results demonstrated that administration of (pyrrolidin-2-yl)methyl 2-(6-methoxy-2-naphthyl) propanoate hydrochloride and 2-(diethylamino)ethyl 2-(4-isobutylphenyl) propanoate hydrochloride protected hACE2 mice from death and lung damages, delayed and reduced the severity of clinical signs of illness.

### Example 71. Efficacy of (Pyrrolidin-2-Yl) Methyl 5-Benzoyl-2,3-Dihydro-1H-Pyrrolizine-1-Carboxylate Hydrochloride and (Pyrrolidin-2-Yl) Methyl 1,8-Diethyl-1,3,4,9-Tetrahydropyrano[3,4-b]Indole-1-Acetate Hydrochloride in Treatment of Signs, Symptoms, and/or Complications of COVID-19 Infection

A total of seventy hACE2 mice (7 groups, 10/group) were used in this study. At day 0, mice were anesthetized with isoflurane and held in a dorsal recumbent position for administration of 3×10 PFU/mouse COVID-19 by internasal instillation, from day 1 post challenge, mice were received a single dose of different concentrations of test article or vehicle control once a day up to 15th day with dose initiation on day 1 post challenge. Group 1 served as an infected control group and was treated with vehicle (35% ethanol). 7% of (pyrrolidin-2-yl)methyl 5-benzoyl-2,3-dihydro-1H-pyrrolizine-1-carboxylate hydrochloride in 35% ethanol (low dose: 24.9 mg/kg, group 2, mid dose: 49.9 mg/kg, group 3, and high dose: 99.7 mg/kg, group 4) and 7% of (pyrrolidin-2-yl)methyl 1,8-diethyl-1,3,4,9-tetrahydropyrano[3,4-b]indole-1-acetate hydrochloride in 35% ethanol (low dose: 24.7 mg/kg, group 5, mid dose: 49.4 mg/kg, group 6, and high dose: 98.9 mg/kg, group 7) and vehicle (35% ethanol) were given by transdermal on the back of animal in an area of 2 cm×5 cm, 1.5 cm×3.5 cm, or 1 cm×2.5 cm for high, middle or low dose. Mice were observed following viral challenge for clinical signs of illness including body weights, health score, morbidity and mortality.

### Example 72. Efficacy of (Pyrrolidin-2-Yl) Methyl 2-13-Chloro-4-(3-Pyrrolin-1-Yl)Phenyl]Propanoate Hydrochloride and (Pyrrolidin-2-Yl) Methyl 2-(5H-[1]-Benzopyrano[2,3-b]Pyridin-7-Yl) Propionate Hydrochloride in Treatment of Signs, Symptoms, and/or Complications of COVID-19 Infection

A total of seventy hACE2 mice (7 groups, 10/group) were used in this study. At day 0, mice were anesthetized with isoflurane and held in a dorsal recumbent position for administration of 3×10 PFU/mouse COVID-19 by internasal instillation, from day 1 post challenge, mice were received a single dose of different concentrations of test article or vehicle control once a day up to 15th day with dose initiation on day 1 post challenge. Group 1 served as an infected control group and was treated with vehicle (35% ethanol). 7% (pyrrolidin-2-yl)methyl 2-[3-chloro-4-(3-pyrrolin-1-yl)phenyl]propanoate hydrochloride in 35% ethanol (low dose: 24.9 mg/kg, group 2, mid dose: 49.9 mg/kg, group 3, and high dose: 99.8 mg/kg, group 4) and 7% (pyrrolidin-2-yl)methyl 2-(5H-[1]-Benzopyrano[2,3-b]pyridin-7-yl) propionate hydrochloride in 35% ethanol (low dose: 24.9 mg/kg, group 5, mid dose: 49.8 mg/kg, group 6, and high dose: 99.7 mg/kg, group 7) and vehicle (35% ethanol) were given by transdermal on the back of animal in an area of 2 cm×5 cm, 1.5 cm×3.5 cm, or 1 cm×2.5 cm for high, middle or low dose. Mice were observed following viral challenge for clinical signs of illness including body weights, health score, morbidity and mortality.

### Example 73. Efficacy of Pyrrolidin-2-Yl) Methyl 4,5-Diphenyl-2—Oxazolepropanoate Hydrochloride and (Pyrrolidin-2-Yl) Methyl 2-(3-Benzoylphenyl) Propionate Hydrochloride in Treatment of Signs, Symptoms, and/or Complications of COVID-19 Infection

A total of seventy hACE2 mice (7 groups, 10/group) were used in this study. At day 0, mice were anesthetized with isoflurane and held in a dorsal recumbent position for administration of 3×10 PFU/mouse COVID-19 by internasal instillation, from day 1 post challenge, mice were received a single dose of different concentrations of test article or vehicle control once a day up to 15th day with dose initiation on day 1 post challenge. Group 1 served as an infected control group and was treated with vehicle (35% ethanol). 7% pyrrolidin-2-yl)methyl 4,5-diphenyl-2-oxazolepropanoate hydrochloride in 35% ethanol (low dose: 24.7 mg/kg, group 2, mid dose: 49.4 mg/kg, group 3, and high dose: 98.7 mg/kg, group 4) and 7% of (pyrrolidin-2-yl)methyl 2-(3-benzoylphenyl) propionate hydrochloride in 35% ethanol (low dose: 24.9 mg/kg, group 5, mid dose: 49.9 mg/kg, group 6, and high dose: 99.7 mg/kg, group 7) and vehicle (35% ethanol) were given by transdermal on the back of animal in an area of 2 cm×5 cm, 1.5 cm×3.5 cm, or 1 cm×2.5 cm for high, middle or low dose. Mice were observed following viral challenge for clinical signs of illness including body weights, health score, morbidity and mortality.

### Example 74. Efficacy of 2-(Diethylamino)Ethyl 2-Acetoxybenzoate Hydrochloride and (Pyrrolidin-2-Yl) Methyl 2-(2-(2,6-Dichlorophenylamino)Phenyl) Acetate Hydrochloride in Cecal Slurry-Induced Sepsis

A total of one hundred (100) C57/BL6 mice were used in this study. Five (5) groups of six (6) were used for cecal slurry titration study and seven (7) groups of ten (10) were used for efficacy in the relief of signs and symptoms of cecal slurry-induced sepsis of test article study. All mice were challenged with cecal slurry by intraperitoneal (IP) injections on Study Day 0. Each mouse received intraperitoneal (IP) injections at doses of 5 mg/mouse, 7.5 mg/mouse, 10 mg/mouse, 12.5 mg/mouse, or 15 mg/mouse decal slurry in the titration study. In efficacy study, at day 0, mice were anesthetized with isoflurane and held in a dorsal recumbent position for administration of 10 mg/mouse cecal slurry by intraperitoneal (IP) injections, from hour 2 post challenge, mice were received a single dose of different concentrations of test article or vehicle control once a day up to 15th day with dose initiation on hour 2 post challenge. Group 1 served as an infected control group and was treated with vehicle (25% ethanol). 7% 2-(diethylamino)ethyl 2-acetoxybenzoate hydrochloride in 25% ethanol (low dose: 25.2 mg/kg, group 2, mid dose: 50.4 mg/kg, group 3, and high dose: 100.8 mg/kg, group 4), and 6.25% (pyrrolidin-2-yl)methyl 2-(2-(2,6-dichlorophenylamino)phenyl) acetate hydrochloride in 25% ethanol (low dose: 22.5 mg/kg, group 5, mid dose: 45 mg/kg, group 6, and high dose: 90 mg/kg, group 7) and vehicle (25% ethanol) were given by transdermal on the back of animal in an area of 2 cm×5 cm, 1.5 cm×3.5 cm, or 1 cm×2.5 cm for high, middle or low dose. Mice were observed following viral challenge for clinical signs of illness including body weights, health score, morbidity and mortality.

### Mortality

For efficacy study the survival of C57/BL6 mice challenged with cecal slurry. 100.8 mg/kg (high dose, group 4) of 2-(diethylamino)ethyl 2-acetoxybenzoate hydrochloride provided the greatest amount of protection with 80% of the animals surviving, 40% of survival were obtained with 25.2 mg/kg (low dose, group 2) and 60% of survival were obtained with 50.4 mg/kg (mid dose, group 3) of 2-(diethylamino)ethyl 2-acetoxybenzoate hydrochloride, 70% of survival was obtained with 22.5 mg/kg (low dose, group 5) and 45 mg/kg (mid dose, group 6) of (pyrrolidin-2-yl)methyl 2-(2-(2,6-dichlorophenylamino)phenyl) acetate hydrochloride, 50% of survival was obtained with 90 mg/kg (high dose, group 7) (pyrrolidin-2-yl)methyl 2-(2-(2,6-dichlorophenylamino)phenyl) acetate hydrochloride, and 0% of survival was obtained with vehicle (25% ethanol, group 1) as shown in Table 18.

TABLE 18
Effect of drug on survival.

| Group No. | Group 1 | Group 2 | Group 3 | Group 4 | Group 5 | Group 6 | Group 7 |
|---|---|---|---|---|---|---|---|
| Animals | 10/10 | 6/10 | 4/10 | 2/10 | 3/10 | 3/10 | 5/10 |
| died/total | | | | | | | |

### Conclusion

The purpose of this study was to evaluate relief of the signs, symptoms and/or complications of cecal slurry induce sepsis of 2-(diethylamino)ethyl 2-acetoxybenzoate hydrochloride and (pyrrolidin-2-yl)methyl 2-(2-(2,6-dichlorophenylamino)phenyl) acetate hydrochloride by transdermal administration on the back of animal (in an area of 2 cm×5 cm, 1.5 cm×3.5 cm, or 1 cm×2.5 cm for high, middle or low doses) in a mouse model of cecal slurry induce sepsis. Results demonstrated that administration of 2-(diethylamino)ethyl 2-acetoxybenzoate hydrochloride and (pyrrolidin-2-yl)methyl 2-(2-(2,6-dichlorophenylamino)phenyl) acetate hydrochloride protected mice from death and delayed and reduced the severity of clinical signs of illness.

### Example 75. Efficacy of (Pyrrolidin-2-Yl)Methyl 2-(3-Phenoxyphenyl) Propionate Hydrochloride and 2-(Diethylamino)Ethyl 2-(4-Isobutylphenyl) Propanoate Hydrochloride in Cecal Slurry-Induced Sepsis

A total of seventy C57/BL6 mice (7 groups, 10/group) were used in this study. At day 0, mice were anesthetized with isoflurane and held in a dorsal recumbent position for administration of 10 mg/mouse cecal slurry by intraperitoneal (IP) injections, from hour 2 post challenge, mice were received a single dose of different concentrations of test article or vehicle control once a day up to 15th day with dose initiation on hour 2 post challenge. Group 1 served as an infected control group and was treated with vehicle (35% ethanol). 7% (pyrrolidin-2-yl)methyl 2-(3-phenoxyphenyl) propionate hydrochloride in 35% ethanol (low dose: 25.1 mg/kg, group 2, mid dose: 50.2 mg/kg, group 3, and high dose: 100.3 mg/kg, group 4) and 7% of 2-(diethylamino)ethyl 2-(4-isobutylphenyl) propanoate hydrochloride in 35% ethanol (low dose: 24.6 mg/kg, group 5, mid dose: 49.2 mg/kg, group 6, and high dose: 98.4 mg/kg, group 7) and vehicle (35% ethanol) were given by transdermal on the back of animal in an area of 2 cm×5 cm, 1.5 cm×3.5 cm, or 1 cm×2.5 cm for high, middle or low dose. Mice were observed following viral challenge for clinical signs of illness including body weights, health score, morbidity and mortality.

### Mortality

0% of survival was obtained with vehicle (35% ethanol, group 1), 10% of survival was obtained with 25.1 mg/kg (group 2) of (pyrrolidin-2-yl)methyl 2-(3-phenoxyphenyl) propionate hydrochloride, 30% of survival was obtained with 50.2 mg/kg (group 3) of (pyrrolidin-2-yl)methyl 2-(3-phenoxyphenyl) propionate hydrochloride, 40% of survival was obtained with 100.3 mg/kg (group 4) of (pyrrolidin-2-yl)methyl 2-(3-phenoxyphenyl) propionate hydrochloride, 50% of survival were obtained with 24.6 mg/kg (group 5) of 2-(diethylamino)ethyl 2-(4-isobutylphenyl) propanoate hydrochloride, 50% of survival was obtained with 49.2 mg/kg (group 6) of 2-(diethylamino)ethyl 2-(4-isobutylphenyl) propanoate hydrochloride, 40% of survival was obtained with 98.4 mg/kg (group 7) 2-(diethylamino)ethyl 2-(p-isobutylphenyl) propanoate hydrochloride as shown in Table 19.

TABLE 19
Effect of drug on survival.

| Group No. | Group 1 | Group 2 | Group 3 | Group 4 | Group 5 | Group 6 | Group 7 |
|---|---|---|---|---|---|---|---|
| Animals | 10/10 | 9/10 | 7/10 | 6/10 | 5/10 | 5/10 | 6/10 |
| died/total | | | | | | | |

### Conclusion

The purpose of this study was to evaluate relief of the signs, symptoms and/or complications of cecal slurry induce sepsis of (pyrrolidin-2-yl)methyl 2-(3-phenoxyphenyl) propionate hydrochloride and 2-(diethylamino)ethyl 2-(p-isobutylphenyl) propanoate hydrochloride by transdermal administration on the back of animal (in an area of 2 cm×5 cm, 1.5 cm×3.5 cm, or 1 cm×2.5 cm for high, middle or low dose) in a mouse model of cecal slurry induce sepsis. Results demonstrated that administration of (pyrrolidin-2-yl)methyl 2-(3-phenoxyphenyl) propionate hydrochloride and 2-(diethylamino)ethyl 2-(p-isobutylphenyl) propanoate hydrochloride protected C57/BL6 mice from death and delayed and reduced the severity of clinical signs of illness.

### Example 76. Efficacy of (Pyrrolidin-2-Yl)Methyl 2-[[2-1 (2,6~Dichlorophenyl)Amino]Phenyl]Acetoxy]Acetate Hydrochloride and (Pyrrolidin-2-Yl)Methyl 1-Methyl-5-(4-Methylbenzoyl)-1H-Pyrrole-2-Acetate Hydrochloride in Cecal Slurry-Induced Sepsis

A total of seventy C57/BL6 mice (7 groups, 10/group) were used in this study. At day 0, mice were anesthetized with isoflurane and held in a dorsal recumbent position for administration of 10 mg/mouse cecal slurry by internasal instillation, from hour 2 post challenge, mice were received a single dose of different concentrations of test article or vehicle control once a day up to 15th day with dose initiation on hour 2 post challenge. Group 1 served as an infected control group and was treated with vehicle (35% ethanol). 7% of (pyrrolidin-2-yl)methyl 2-[[2-[(2,6-dichlorophenyl)amino]phenyl]acetoxy]acetate hydrochloride in 35% ethanol (low dose: 24.9 mg/kg, group 2, mid dose: 49.9 mg/kg, group 3, and high dose: 99.7 mg/kg, group 4) and 7% of (pyrrolidin-2-yl)methyl 1-methyl-5-(4-methylbenzoyl)-1H-pyrrole-2-acetate hydrochloride in 35% ethanol (low dose: 24.7 mg/kg, group 5, mid dose: 49.4 mg/kg, group 6, and high dose: 98.9 mg/kg, group 7) and vehicle (35% ethanol) were given by transdermal on the back of animal in an area of 2 cm×5 cm, 1.5 cm×3.5 cm, or 1 cm×2.5 cm for high, middle or low dose. Mice were observed following viral challenge for clinical signs of illness including body weights, health score, morbidity and mortality.

### Example 77. Efficacy of (Pyrrolidin-2-Yl)Methyl 5-(4-Chlorobenzoyl)-1,4-Dimethyl-1H-Pyrrole-2-Acetate Hydrochloride and (Pyrrolidin-2-Yl)Methyl Dichlorophenoxy)Benzeneacetate Hydrochloride in Cecal Slurry-Induced Sepsis

A total of seventy C57/BL6 mice (7 groups, 10/group) were used in this study. At day 0, mice were anesthetized with isoflurane and held in a dorsal recumbent position for administration of 1 μm/kg cecal slurry by internasal instillation, from hour 2 post challenge, mice were received a single dose of different concentrations of test article or vehicle control once a day up to 15th day with dose initiation on hour 2 post challenge. Group 1 served as an infected control group and was treated with vehicle (35% ethanol). 7% of (pyrrolidin-2-yl)methyl 5-(4-chlorobenzoyl)-1,4-dimethyl-1H-pyrrole-2-acetate hydrochloride in 35% ethanol (low dose: 24.9 mg/kg, group 2, mid dose: 49.9 mg/kg, group 3, and high dose: 99.7 mg/kg, group 4) and 7% of (pyrrolidin-2-yl)methyl dichlorophenoxy)benzeneacetate hydrochloride in 35% ethanol (low dose: 24.7 mg/kg, group 5, mid dose: 49.4 mg/kg, group 6, and high dose: 98.9 mg/kg, group 7) and vehicle (35% ethanol) were given by transdermal on the back of animal in an area of 2 cm×5 cm, 1.5 cm×3.5 cm, or 1 cm×2.5 cm for high, middle or low dose. Mice were observed following viral challenge for clinical signs of illness including body weights, health score, morbidity and mortality.

### Example 78. Efficacy of 2-(Diethylamino)Ethyl 2-Acetoxybenzoate Hydrochloride and (Pyrrolidin-2-Yl)Methyl 2-(2-(2,6-Dichlorophenylamino)Phenyl) Acetate Hydrochloride in Sepsis Model of MRSA Infection

A total of one hundred (100) C57/BL6 mice were used in this study. Six (6) groups of five (5) were used for Methicillin-resistant S. aureus strain NRS71 (Sangar 252) titration study and seven (7) groups of ten (10) were used for efficacy in the relief of signs and symptoms of sepsis model of MRSA infection of test article study. All mice were challenged with Methicillin-resistant S. aureus strain NRS71 (Sangar 252) by intraperitoneal (IP) injections on Study Day 0. Each mouse received intraperitoneal (IP) injections at doses of 4×10⁶. CFU S. aureus, 2×10⁷. CFU S. aureus, 1×10⁸. CFU S. aureus, 5 ×10⁸. CFU S. aureus, 1×10⁹ CFU S. aureus, and 2×10⁹ CFU S. aureus in the titration study. In efficacy study, at day 0, mice were anesthetized with isoflurane and held in a dorsal recumbent position for administration of 2×10⁷ CFU S. aureus by intraperitoneal (IP) injections, from hour 2 post challenge, mice were received a single dose of different concentrations of test article or vehicle control once a day up to 15th day with dose initiation on hour 2 post challenge. Group 1 served as an infected control group and was treated with vehicle (25% ethanol). 7% 2-(diethylamino)ethyl 2-acetoxybenzoate hydrochloride in 25% ethanol (low dose: 25.2 mg/kg, group 2, mid dose: 50.4 mg/kg, group 3, and high dose: 100.8 mg/kg, group 4), and 6.25% (pyrrolidin-2-yl)methyl 2-(2-(2,6-dichlorophenylamino)phenyl) acetate hydrochloride in 25% ethanol (low dose: 22.5 mg/kg, group 5, mid dose: 45 mg/kg, group 6, and high dose: 90 mg/kg, group 7) and vehicle (25% ethanol) were given by transdermal on the back of animal in an area of 2 cm×5 cm, 1.5 cm×3.5 cm, or 1 cm×2.5 cm for high, middle or low dose. Mice were observed following MRSA challenge for clinical signs of illness including body weights, health score, morbidity and mortality.

### Mortality

For efficacy study the survival of C57/BL6 mice challenged with MRSA is presented in Table 20. 100.8 mg/kg (high dose, group 4) of 2-(diethylamino)ethyl 2-acetoxybenzoate hydrochloride provided the greatest amount of protection with 50% of the animals surviving, 20% of survival were obtained with 25.2 mg/kg (low dose, group 2) and 30% of survival were obtained with 50.4 mg/kg (mid dose, group 3) of 2-(diethylamino)ethyl 2-acetoxybenzoate hydrochloride, 40% of survival was obtained with 22.5 mg/kg (low dose, group (pyrrolidin-2-yl)methyl 2-(2-(2,6-5) of dichlorophenylamino)phenyl) acetate hydrochloride, 60% of survival was obtained with 45 mg/kg (mid dose, group 6 of (pyrrolidin-2-yl)methyl 2-(2-(2,6-dichlorophenylamino)phenyl) acetate hydrochloride, 50% of survival was obtained with 90 mg/kg (high dose, group 7) (pyrrolidin-2-yl)methyl 2-(2-(2,6-dichlorophenylamino)phenyl) acetate hydrochloride, and 0% of survival was obtained with vehicle (25% ethanol, group 1) as shown in Table 20.

TABLE 20
Effect of drug on survival.

| Group No. | Group 1 | Group 2 | Group 3 | Group 4 | Group 5 | Group 6 | Group 7 |
|---|---|---|---|---|---|---|---|
| Animals | 10/10 | 8/10 | 7/10 | 5/10 | 6/10 | 4/10 | 5/10 |
| died/total | | | | | | | |

### Conclusion

The purpose of this study was to evaluate relief of the signs, symptoms and/or complications of MRSA induced sepsis of 2-(diethylamino)ethyl 2-acetoxybenzoate hydrochloride and (pyrrolidin-2-yl)methyl 2-(2-(2,6-dichlorophenylamino)phenyl) acetate hydrochloride by transdermal administration on the back of animal (in an area of 2 cm×5 cm, 1.5 cm×3.5 cm, or 1 cm×2.5 cm for high, middle or low dose) in a mouse model of MRSA induced sepsis. Results demonstrated that administration of 2-(diethylamino)ethyl 2-acetoxybenzoate hydrochloride and (pyrrolidin-2-yl)methyl 2-(2-(2,6-dichlorophenylamino)phenyl) acetate hydrochloride protected mice from death and delayed and reduced the severity of clinical signs of illness.

### Example 79. Efficacy of (Pyrrolidin-2-Yl)Methyl 2-(6-Methoxy-2-Naphthyl) Propanoate Hydrochloride and 2-(Diethylamino)Ethyl 2-(4-Isobutylphenyl) Propanoate Hydrochloride in Sepsis Model of MRSA Infection

A total of seventy C57/BL6 mice (7 groups, 10/group) were used in this study. At day 0, mice were anesthetized with isoflurane and held in a dorsal recumbent position for administration of 2×10⁷CFU S. aureus by intraperitoneal (IP) injections, from hour 2 post challenge, mice were received a single dose of different concentrations of test article or vehicle control once a day up to 15th day with dose initiation on hour 2 post challenge. Group 1 served as an infected control group and was treated with vehicle (35% ethanol). 7% of (pyrrolidin-2-yl)methyl 2-(6-methoxy-2-naphthyl) propanoate hydrochloride in 35% ethanol (low dose: 25.1 mg/kg, group 2, mid dose: 50.2 mg/kg, group 3, and high dose: 100.3 mg/kg, group 4) and 7% of 2-(diethylamino)ethyl 2-(4-isobutylphenyl) propanoate hydrochloride in 35% ethanol (low dose: 24.6 mg/kg, group 5, mid dose: 49.2 mg/kg, group 6, and high dose: 98.4 mg/kg, group 7) and vehicle (35% ethanol) were given by transdermal on the back of animal in an area of 2 cm×5 cm, 1.5 cm×3.5 cm, or 1 cm×2.5 cm for high, middle or low dose. Mice were observed following MRSA challenge for clinical signs of illness including body weights, health score, morbidity and mortality.

### Mortality

0% of survival was obtained with vehicle (35% ethanol, group 1), 20% of survival was obtained with 25.1 mg/kg (group 2) of (pyrrolidin-2-yl)methyl 2-(6-methoxy-2-naphthyl) propanoate hydrochloride, 30% of survival was obtained with 50.2 mg/kg (group 3) of (pyrrolidin-2-yl)methyl 2-(6-methoxy-2-naphthyl) propanoate hydrochloride, 40% of survival was obtained with 100.3 mg/kg (group 4) of (pyrrolidin-2-yl)methyl 2-(6-Methoxy-2-naphthyl) propanoate hydrochloride, 40% of survival were obtained with 24.6 mg/kg (group 5) of 2-(diethylamino)ethyl 2-(4-isobutylphenyl) propanoate hydrochloride, 60% of survival was obtained with 49.2 mg/kg (group 6) of 2-(diethylamino)ethyl 2-(4-isobutylphenyl) propanoate hydrochloride, 50% of survival was obtained with 98.4 mg/kg (group 7) 2-(diethylamino)ethyl 2-(4-isobutylphenyl) propanoate hydrochloride as shown in Table 21.

TABLE 21
Effect of drug on survival.

| Group No. | Group 1 | Group 2 | Group 3 | Group 4 | Group 5 | Group 6 | Group 7 |
|---|---|---|---|---|---|---|---|
| Animals | 10/10 | 8/10 | 7/10 | 6/10 | 6/10 | 4/10 | 5/10 |
| died/total | | | | | | | |

### Conclusion

The purpose of this study was to evaluate relief of the signs, symptoms and/or complications of MRSA induced sepsis of (pyrrolidin-2-yl)methyl 2-(6-methoxy-2-naphthyl) propanoate hydrochloride and 2-(diethylamino)ethyl 2-(4-isobutylphenyl) propanoate hydrochloride by transdermal administration on the back of animal (in an area of 2 cm×5 cm, 1.5 cm×3.5 cm, or 1 cm×2.5 cm for high, middle or low doses) in a mouse model of MRSA induced sepsis. Results demonstrated that administration of (pyrrolidin-2-yl)methyl 2-(6-methoxy-2-naphthyl) propanoate hydrochloride and 2-(diethylamino)ethyl 2-(4-isobutylphenyl) propanoate hydrochloride protected C57/BL6 mice from death and delayed and reduced the severity of clinical signs of illness.

### Example 80. Efficacy of (Pyrrolidin-2-Yl)Methyl 5-Benzoyl-2,3-Dihydro-1H-Pyrrolizine-1-Carboxylate Hydrochloride and (Pyrrolidin-2-Yl)Methyl 1,8-Diethyl-1,3,4,9-Tetrahydropyrano[3,4-b]Indole-1-Acetate Hydrochloride in Sepsis Model of MRSA Infection

A total of seventy C57/BL6 mice (7 groups, 10/group) were used in this study. At day 0, mice were anesthetized with isoflurane and held in a dorsal recumbent position for administration of 2×10⁷ CFU S. aureus by internasal instillation, from hour 2 post challenge, mice were received a single dose of different concentrations of test article or vehicle control once a day up to 15th day with dose initiation on hour 2 post challenge. Group 1 served as an infected control group and was treated with vehicle (35% ethanol). 7% of (pyrrolidin-2-yl)methyl 5-benzoyl-2,3-dihydro-1H-pyrrolizine-1-carboxylate hydrochloride in 35% ethanol (low dose: 24.9 mg/kg, group 2, mid dose: 49.9 mg/kg, group 3, and high dose: 99.7 mg/kg, group 4) and 7% of (pyrrolidin-2-yl)methyl 1,8-diethyl-1,3,4,9-tetrahydropyrano[3,4-b]indole-1-acetate hydrochloride in 35% ethanol (low dose: 24.7 mg/kg, group 5, mid dose: 49.4 mg/kg, group 6, and high dose: 98.9 mg/kg, group 7) and vehicle (35% ethanol) were given by transdermal on the back of animal in an area of 2 cm×5 cm, 1.5 cm×3.5 cm, or 1 cm×2.5 cm for high, middle or low dose. Mice were observed following MRSA challenge for clinical signs of illness including body weights, health score, morbidity and mortality.

### Example 81. Efficacy of 2-(Diethylamino)Ethyl 2-Acetoxybenzoate Hydrochloride in a Phase 2, Multicenter, Randomized, Double-Blind, Placebo-Controlled, Parallel-Group, and Dose-Range-Finding Study in Treatments for COVID-19 Infection in Hospitalized Adults

Patients cannot be enrolled or randomized before all eligibility criteria (including test results) are confirmed.

Inclusion criteria:
1. Admitted to a hospital with pulmonary symptoms of active COVID-19.
2. Patient (or legally authorized representative) provides informed consent prior to initiation of any study procedures.
3. Patient (or legally authorized representative) understands and agrees to comply with planned study procedures.
4. Male or nonpregnant female adult ≥18 years of age at time of enrollment.
5. Has laboratory confirmed severe acute respiratory syndrome coronavirus 2 (COVID-19) infection as determined by polymerase chain reaction (PCR) or other commercial or public health assay (serology is not acceptable) in any specimen, as documented by either of the following:
   a. Laboratory confirmed COVID-19 infection by method as defined above, in sample collected <72 hours prior to randomization; OR
   b. Laboratory confirmed COVID-19 infection by method as defined above, in sample collected ≥72 hours prior to randomization, documented inability to obtain a repeat sample (e.g., due to lack of testing supplies, limited testing capacity, results taking >24 hours, etc.) AND progressive disease suggestive of ongoing COVID-19 infection.
6. Has a room-air SpO₂<93% and a score of 4 (hospitalized, oxygen by mask or nasal prongs), or room-air SpO₂<93% and a score of 5 (hospitalized, noninvasive ventilation or high-flow oxygen), or a score 6 (hospitalized, intubation and mechanical ventilation) in the ordinal scale, which specifically is as follows:
   0) Uninfected, no clinical or virological evidence of infection
   1) Ambulatory (not diagnosed), no limitations of activities.
   2) Ambulatory (not diagnosed), limitation on activities and/or requiring home oxygen
   3) Diagnosed (mild disease), no requiring supplemental oxygen;
   4) Diagnosed (moderate disease), requiring supplemental oxygen (by mask or nasal prongs)—requiring ongoing medical care (COVID-19 related or otherwise);
   5) Diagnosed (severe disease), non-invasive ventilation or high-flow oxygen;
   6) Diagnosed (severe disease), intubation and mechanical ventilation;
   7) Diagnosed (severe disease), ventilation+additional organ support-pressors, RRT, or extracorporeal membrane oxygenation (ECMO);
   8) Death.
7. Women of childbearing potential and males must agree to either abstinence or use at least 1 primary form of contraception not including hormonal contraception from the time of screening through Day 37. (e.g. Double-barrier method [condoms, sponge, diaphragm, with spermicidal jellies, or cream], same-sex partner, surgically sterilized patient/partner [>6 months post sterilization]are acceptable).
8. Agrees to not join a new interventional clinical study through Day 60 (or 30 days after the last dose of study drug). However, Food and Drug Administration (FDA)-approved or under an emergency use authorization antivirus or other drugs may be allowed, and that should be judged by the patient's physician.

Exclusion criteria:
1. Any condition or reason, including any significant medical or neuropsychiatric condition, including the presence of laboratory abnormalities, including local or regional requirements to be eligible to participate in a clinical study, which in the judgment of the investigator places the patient at unacceptable risk if he/she were to participate in the study, violates local or regional law/requirements, or confounds the ability to interpret data from the study including, but not limited to:
   a. Platelet count <50× 10⁹/L.
   b. Does not meet local or regional requirements to be eligible to participate in a clinical study.
2. Extracorporeal membrane oxygenation required at baseline.
3. Stage 4 or worse chronic kidney disease or end stage renal disease (ESRD).
4. Is at increased risk for bleeding events (e.g., had recent cerebral hemorrhage, gastrointestinal bleeding, serious trauma, recent surgery, or organ biopsy).
5. Currently are using anti-platelet agents (e.x Aspirin).
6. Pregnant (patient has a positive pregnancy test result at screening) or breast-feeding.
7. Anticipated discharge from the hospital or transfer to another hospital which is not a study site.
8. Allergy to any study medication or known allergy to nonsteroidal anti-inflammatory drugs, including aspirin.
9. Patient must agree to refrain from taking oral aspirin or any orally administered acetylsalicylic acid medications until PK sampling is completed to be eligible to participate in the PK sub-study.

Efficacy endpoints:

Primary efficacy endpoint:

The difference of stay time in hospital between the test and the control arm.

Key Secondary endpoint:
Time to patients' ability to maintain peripheral capillary oxygen saturation (SpO₂) of >93% without oxygen support.
   Other Secondary endpoint:
1. Time to an improvement of 1 category using 9-point ordinal scale.
2. Time to an improvement of 2 categories using 9-point ordinal scale.

Dosage regimen:

A 7% solution of 2-(diethylamino)ethyl 2-acetoxybenzoate hydrochlorid in 15% ethanol was used as the drug of the invention, and the vehicle (15% ethanol) was used as placebo.
Each patient or their caregiver should administer the drug as instructed by study personnel. Administration on either the front or back of the patient is acceptable for patients who are not prone. The administration location should be documented in each patient's source and eCRF. When administering the drug on the back, the instructions for prone patients will be followed. When administering IP on the front, the instructions for patients who are not prone will be followed.
For patients who are prone, the drug will be administered as follows:
Low dose group: 1 spray to the skin on the back of the neck, 1 spray to the skin on the left side of the neck, 1 spray to the skin on the right side of the neck, and 9 sprays to the back skin around the lungs (a total of 12 sprays, 84 mg) applied BID for a total of 24 sprays (168 mg)/day, for 28 days.
High dose group: 2 sprays to the skin on the back of the neck, 1 spray to the skin on the left side of the neck, 1 spray to the skin on the right side of the neck, and 20 sprays to the back skin (a total of 24 sprays, 168 mg) applied BID for a total of 48 sprays (336 mg)/day, for 28 days.
   For patients who are not prone, the drug will be administered as follows:
Low dose group: 1 spray to the skin on the front of the neck, 1 spray to the skin on the left side of the neck, 1 spray to the skin on the right side of the neck, and 9 sprays to the chest skin around the lungs (a total of 12 sprays, 84 mg) applied BID for a total of 24 sprays (168 mg)/day, for 28 days
High dose group: 2 sprays to the skin on the front of the neck, 1 spray to the skin on the left side of the neck, 1 spray to the skin on the right side of the neck, and 20 sprays to the chest skin (a total of 24 sprays, 168 mg) applied BID for a total of 48 sprays (336 mg)/day, for 28 days.

Efficacy Results:

Patients who were treated with the drug of the invention have about 4 days in an average in hospital stay time, with an average age of 65.5. Patients who were treated with placebo have about 8 days in an average in hospital stay time, with an average age of 64.1. Some patients who were treated with placebo failed to meet discharge criteria on the 29-day.
Patients who were treated with the drug of the invention needed about 3 days in an average to maintain peripheral capillary oxygen saturation (SpO₂) of >93% without oxygen support. Patients who were treated with placebo needed about 8 days in an average to maintain peripheral capillary oxygen saturation (SpO₂) of >93% without oxygen support. Some patients who were treated with placebo still needed oxygen support on the 29-day.
Patients who were treated with the drug of the invention needed about 3 days in an average to have an improvement of 2 categories using 9-point ordinal scale and patients who were treated with placebo needed about 8 days in an average to have an improvement of 2 categories using 9-point ordinal scale.

The invention claimed is:

1. A method of treating a sign, symptom, and/or complication of a viral, bacterial, protozoal, and/or fungal infection, or a related condition thereof in a subject, comprising administering a high penetration prodrug (HPP) of Structure L-1: 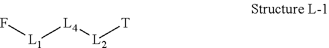
or a stereoisomer or pharmaceutically acceptable salt thereof to the subject, wherein:
F, as a functional unit, is a molecular moiety of an active pharmaceutical ingredient selected from 5-lipoxygenase-activating protein (FLAP) inhibitors, 5-lipoxygenase inhibitors, leukotriene receptor antagonists, and anti-inflammatory agents;
T, as a transportational unit, is a basic group comprising a protonatable nitrogen;
L₁, L₂, and L₄ together form a linker such that the compound of Structure L-1 is capable of being hydrolyzed or metabolized under physiological conditions to release the functional unit F to form the active pharmaceutical ingredient, or a biologically active metabolite thereof.

2. The method according to claim 1, wherein:
L₁ is selected from a bond, O, S, —N(L₃)—, —N(L₃)-CH₂—O, —N(L₃)-CH₂—N(L₃)—, —O—CH₂—O—, —O—CH (L₃)-O, —S—CH(L₃)-O—;
L₂ is selected from a bond, O, S, —N(L₃)—, —N(L₃)-CH₂—O, —N(L₃)-CH₂—N(L₃)—, —O—CH₂—O—, —O—CH(L₃)-O, —S—CH(L₃)—O—, —O-L₅-, —N-L₅-, —S-L₅-, and —N(L₃)-L₅-; 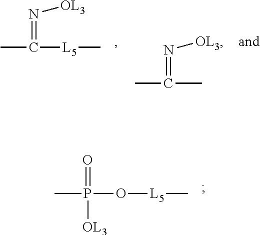
L₄ is selected from a bond, C═O, C═S,
each L₃ is independently H or C₁-C₆ alkyl;
each L₅ is independently selected from a bond, CH₂COOL₆, substituted or unsubstituted C₁-C₆ alkylene, substituted or unsubstituted C₃-C₆ cycloalkylene, substituted and unsubstituted 5- to 10-membered heterocycloalkylene, substituted and unsubstituted C₆-C₁₀ arylene, and substituted and unsubstituted 5- to 10-membered heteroarylene, wherein L₆ is a bond or substituted or unsubstituted C₁-C₆ alkylene;
provided, however, that Structure L-1 forms a stable compound with no covalent bonding principles being violated.

3. The method according to claim 1, wherein:
the linker is selected from —O-X-X₅—, —NH-X-X₅—, —S-X-X₅—, —O—C(═O)—X₅—, —NH—C(═O)—X₅—, —S—C(═O)—X-X₅—, —C(═O)—O—X-X₅—, —C(═O)—S—X, —C(═O)—NH—X—, —X-X₅—C(═O), —C(═O) X-X—, —X₅—C(═O), and C(═O)—X₅—;
X is a bond, C(═O), or C₁-C₄ alkylene; and
X₅ is selected from a bond, C(═O), C₁-C₄ alkylene, S, O, and NR₅; and
R₅ is H or C₁-C₆ alkyl.

4. The method according to claim 1, wherein the linker is selected from C(═O)—O, C(═O)—S, C(═O)—NH, C(═O)—O—CH(L₅)-O, C(═O)—O—CH(L₅)-S, C(═O)—O—CH(L₅)-NH, C(═O)—S—CH(L₅)-O, C(═O)—S—CH(L₅)-S, C(═O)—S—CH(L₅)-NH, P (═O) (—O-L₅)-O, O—P(═O) (—O-L₅), C(═S)—O, C(═NH)—O, C(═NH)—S, C(═N-OL₃)-NH, C(═NH-OL₃)-O, C(═NH-OL₃)-S, O—C(═O), S—C(═O), NH—C(═O), O—CH(L₅)—O—C(═O), S—CH(L₅)—O—C(═O), O—CH(L₅)-S—C(═O), S—CH(L₆)-S—C(═O), O—CH(L₆)-NH—C(═O), O—C(═S), O—C(═N-OL₃), S—C(═N-OL₃), and NH—C(═N-OL₃), wherein L₃ and L₅ at each occurrence is independently selected from H, substituted and unsubstituted alkyl, substituted and unsubstituted cycloalkyl, substituted and unsubstituted heterocyclyl, substituted and unsubstituted aryl, substituted and unsubstituted heteroaryl, substituted and unsubstituted alkoxy, substituted and unsubstituted alkylthio, and substituted and unsubstituted alkylamino.

5. The method according to claim 1, wherein T is a substituted or unsubstituted primary amine, substituted or unsubstituted secondary amine, substituted or unsubstituted tertiary amine, or heterocyclyl group comprising a protonatable nitrogen in the ring.

6. The method according to claim 1, wherein T is selected from Structure W-1, Structure W-2, Structure W-3, Structure W-4, Structure W-5, and Structure W-6: 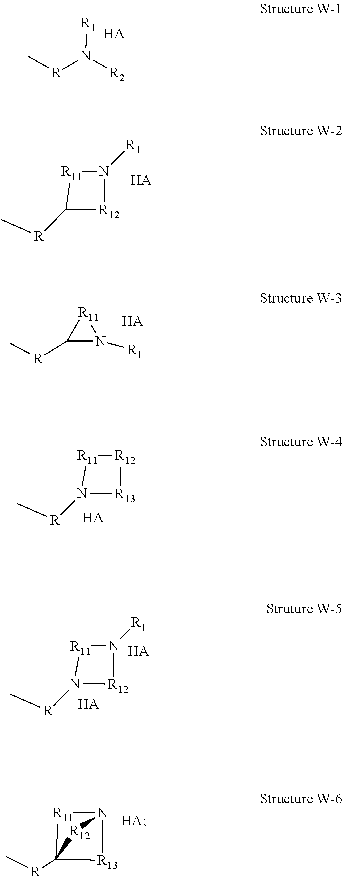
R at each occurrence is independently selected from a bond, substituted and unsubstituted alkylene, substituted and unsubstituted cycloalkylene, substituted and unsubstituted heterocyclylene;
R₁ and R₂ are independently selected from H, substituted and unsubstituted alkyl, substituted and unsubstituted cycloalkyl, and substituted and unsubstituted heterocyclyl; or alternatively R₁ and R₂ together with the nitrogen atom to which they are attached form an optionally substituted heterocyclyl, which optionally further comprises one or two additional heteroatom(s) independently selected from O, S, and N;
R₁₁, R₁₂, and R₁₃ are each independently a bond, an optionally substituted C₁-C₄ alkylene, or an optionally substituted C₂-C₄ alkyenylene, wherein the alkylene and alkenylene optionally has one CH₂ group replaced by O, S, or NR₃;
wherein any of the R₁ in Structure W-2, Structure W-3 or Structure W-5 and the adjacent R₁₁ together with the nitrogen atom to which they are attached may form an optionally substituted heterocyclic ring, which may optionally further comprise one or two additional heteroatom(s) independently selected from O, S, and N; and
wherein the R₁₁ and R₁₂ or R₁₁ and R₁₃ in Structure W-2, Structure W-4, Structure W-5, or Structure W-6 may optionally be connected by an alkylene bridge, which is optionally substituted; and
wherein HA is nothing or a pharmaceutically acceptable acid;
provided, however, that the Structure L-1 forms a stable compound with no covalent bonding principles being violated.

7. The method according to claim 6, wherein R is a bond or C₁-C₆ alkylene.

8. The method according to claim 6, wherein R₁₁, R₁₂, and R₁₃ are each independently selected from CH₂, CH₂CH₂, CH═CH, CH₂CH₂CH₂, CH═CHCH₂, CH₂CH₂CH₂CH₂, CH₂CH═CH—CH₂, CH₂CH₂CH₂CH₂CH₂, CH₂CH₂CH₂CH₂CH₂CH₂, each of which is optionally substituted.

9. The method according to claim 6, wherein the transportational unit T is Structure W-1, wherein R₁ and R₂ are each hydrogen or C₁-C₆ alkyl.

10. The method according to claim 6, wherein the transportational unit T is Structure W-2, Structure W-3, Structure W-4, Structure W-5, or Structure W-6, wherein R is a bond or C₁-C₄ alkylene; R₁ is hydrogen or C₁-C₆ alkyl; R₁₁ is a C₁-C₄ alkylene; R₁₂ and R₁₃ are independently a bond, CH₂, or CH₂CH₂.

11. The method according to claim 1, wherein the transportational unit T is a heterocyclyl selected from pyrrolidinyl, piperidinyl, piperazinyl, and morpholinyl, each of which is protonated with hydrochloride, hydrobromide, or acetic acid.

12. The method according to claim 1, wherein the HPP is selected from Structure LRA-1, Structure LRA-2, Structure LRA-3, Structure LRA-4, Structure LRA-5, Structure LRA-6, Structure ARA-1, Structure ARA-2, Structure ARA-3, Structure ARA-4, Structure ARA-5, Structure ARA-6, Structure ARA-7, Structure ARA-8, Structure ARA-9, Structure ARA-11, Structure ARA-11, Structure ARA-12, Structure ARA-13, Structure ARA-14, Structure 5-LI-1, Structure 5-LI-2, Structure 5-LI-3, Structure 5-LI-4, Structure 5-LI-5, Structure 5-LI-6, Structure 5-LI-7, Structure 5-LI-8, Structure FLAP-1, Structure FLAP-2, Structure FLAP-3, Structure FLAP-4, Structure FLAP-5, Structure FLAP-6, Structure NSAID-1, Structure NSAID-2, Structure NSAID-3, Structure NSAID-4, Structure NSAID-5, Structure NSAID-6, Structure NSAID-7, Structure NSAID-8, Structure NSAID-9, Structure NSAID-10, Structure NSAID-11, Structure NSAID-12, and Structure NSAID-13: 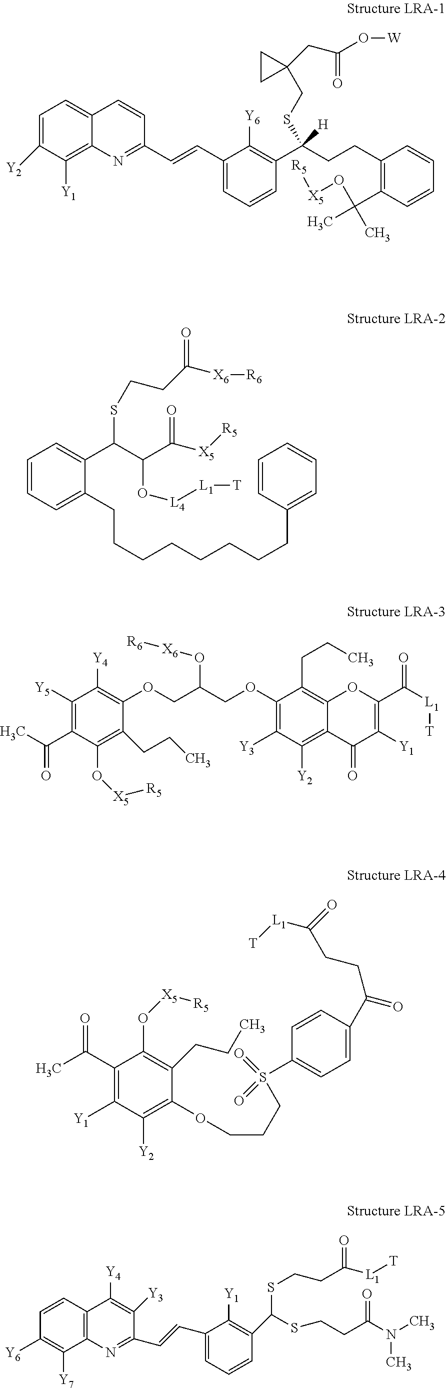 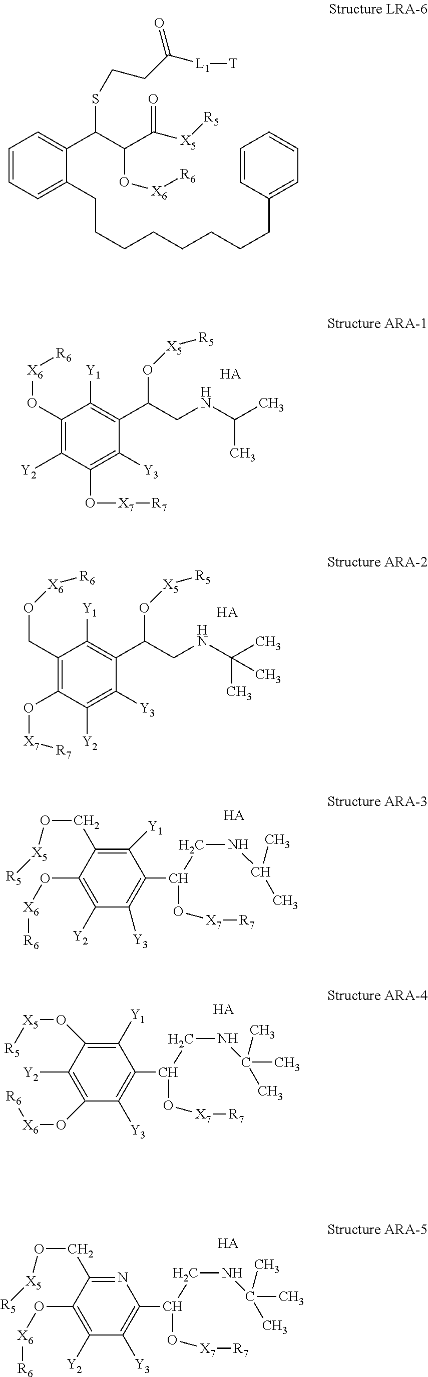 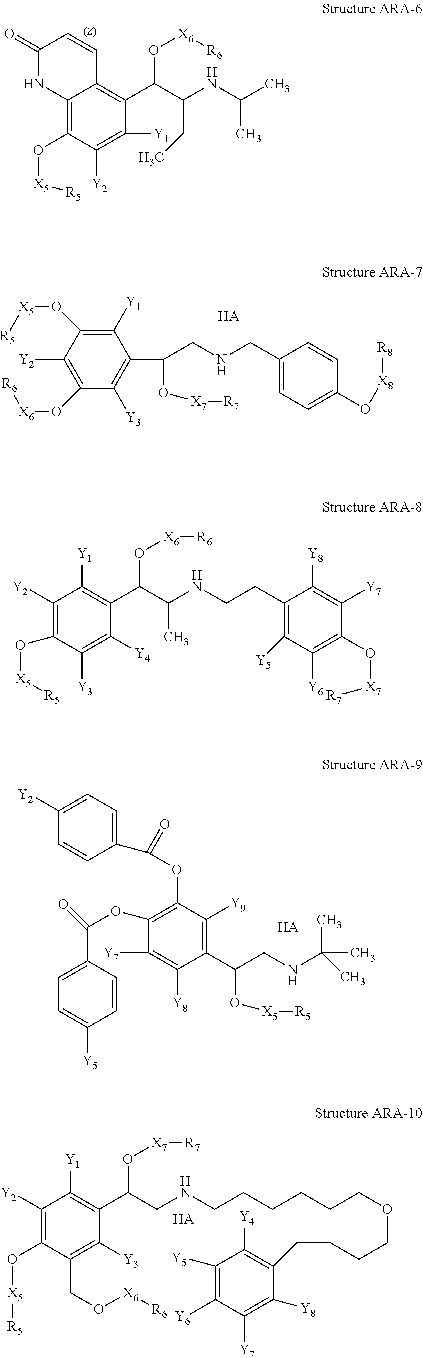 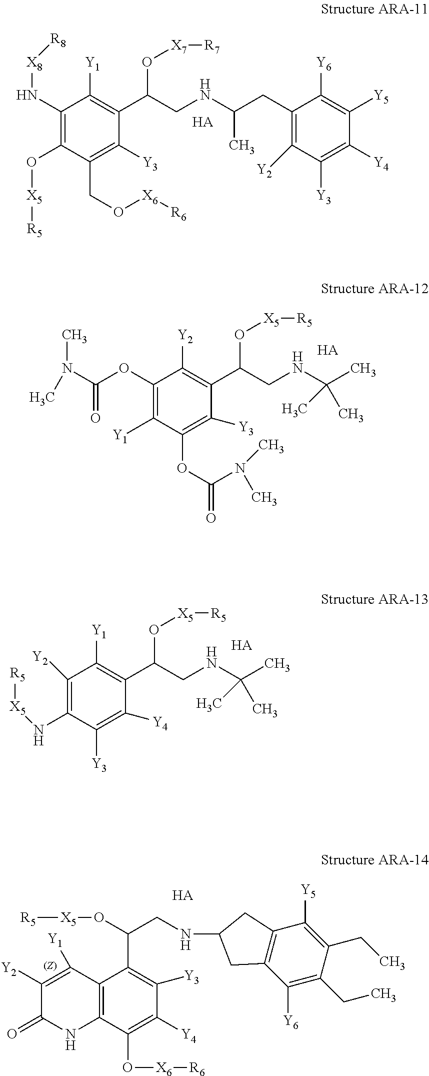 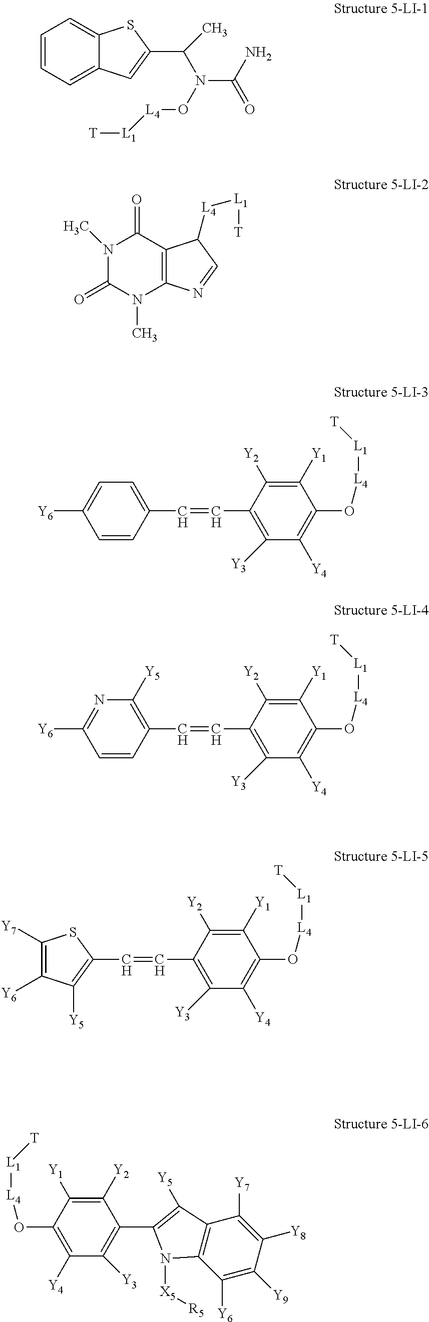 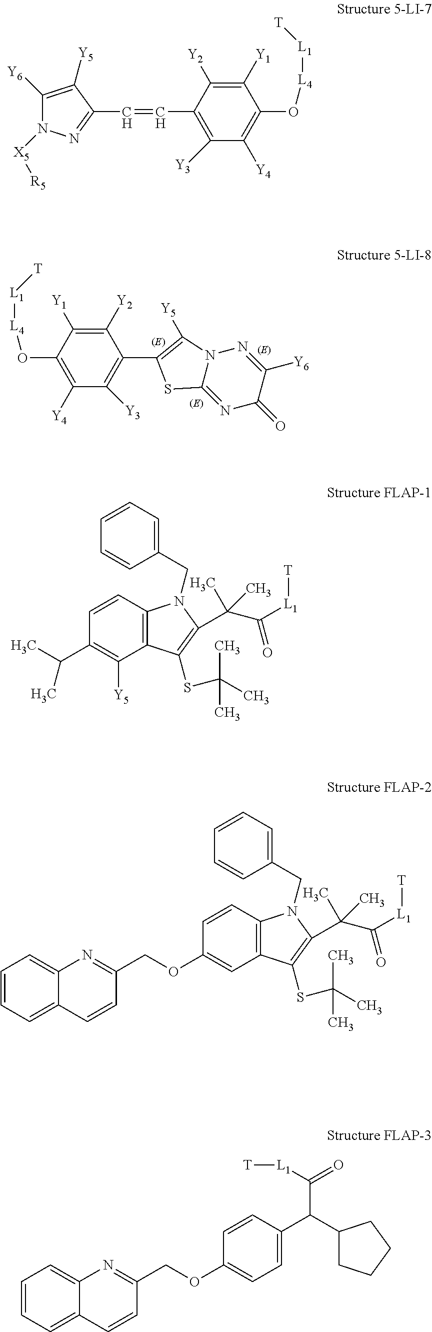 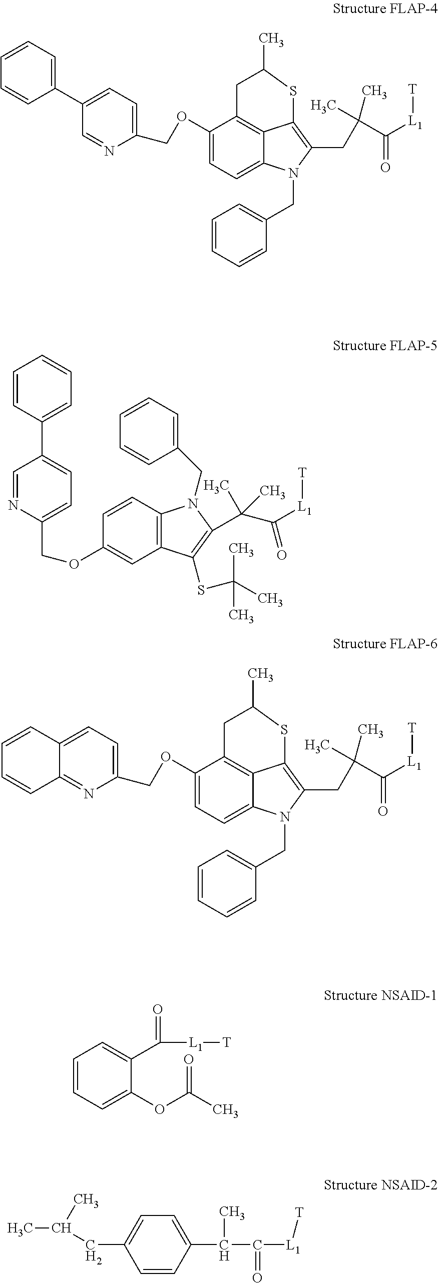 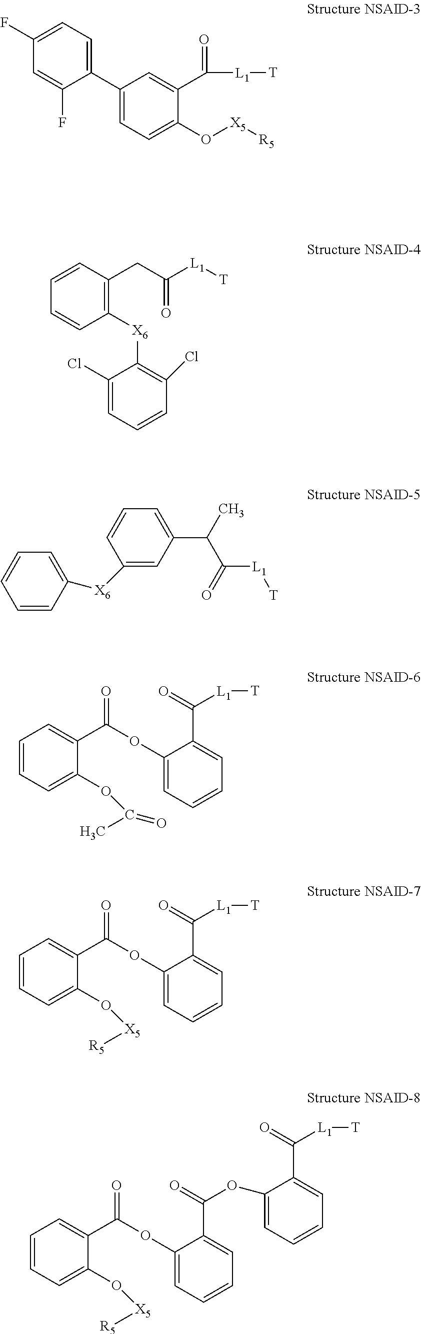 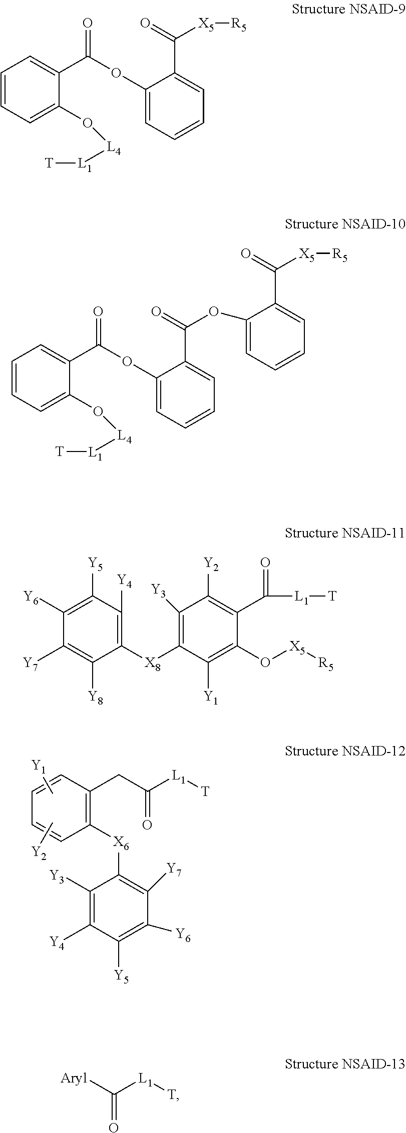
and stereoisomers and pharmaceutically acceptable salts thereof;
wherein Aryl- is a molecular moiety of an anti-inflammatory drug, or an anti-inflammatory drug-related compound, selected from: Aryl-1, Aryl-2, Aryl-3, Aryl-4, Aryl-5, Aryl-6, Aryl-7, Aryl-8, Aryl-9, Aryl-10, Aryl-11, Aryl-12, Aryl-13, Aryl-14, Aryl-15, Aryl-16, Aryl-17, Aryl-18, Aryl-19, Aryl-20, Aryl-21, Aryl-22, Aryl-23, Aryl-24, Aryl-25, Aryl-26, Aryl-27, Aryl-28, Aryl-29, Aryl-30, Aryl-31, Aryl-32, Aryl-33, Aryl-34, Aryl-35, Aryl-36, Aryl-37, Aryl-38, Aryl-39, Aryl-40, Aryl-41, Aryl-42, Aryl-43, Aryl-44, Aryl-45, Aryl-46, Aryl-47, Aryl-48, Aryl-49, Aryl-50, Aryl-51, Aryl-52, Aryl-53, Aryl-54, Aryl-55, Aryl-56, Aryl-57, Aryl-58, Aryl-59, Aryl-60, Aryl-61, Aryl-62, Aryl-63, Aryl-64, Aryl-65, Aryl-66, Aryl-67, Aryl-68, Aryl-69, Aryl-70, and Aryl-71: 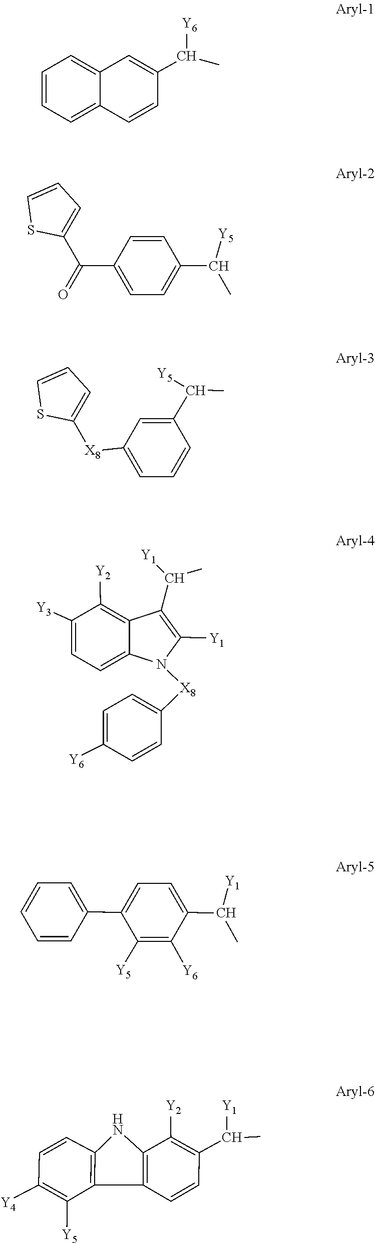 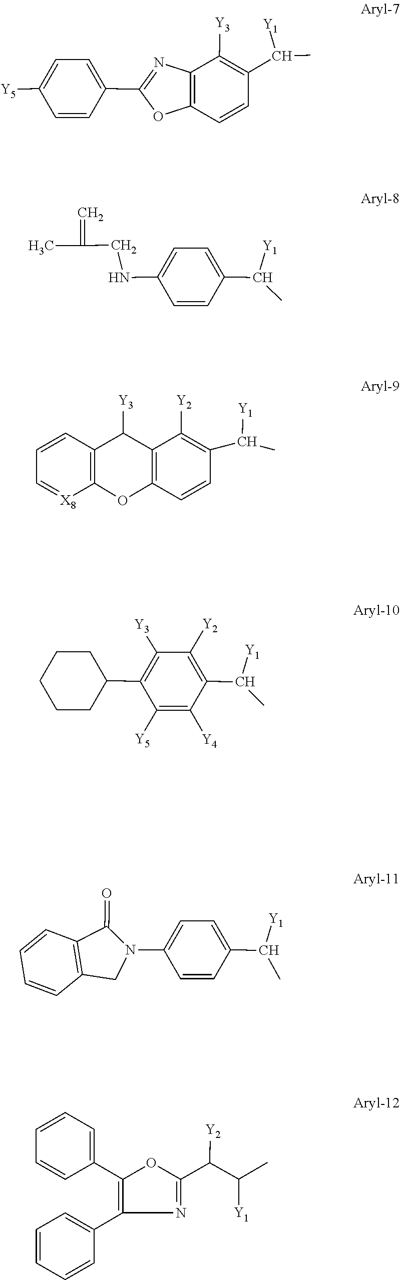 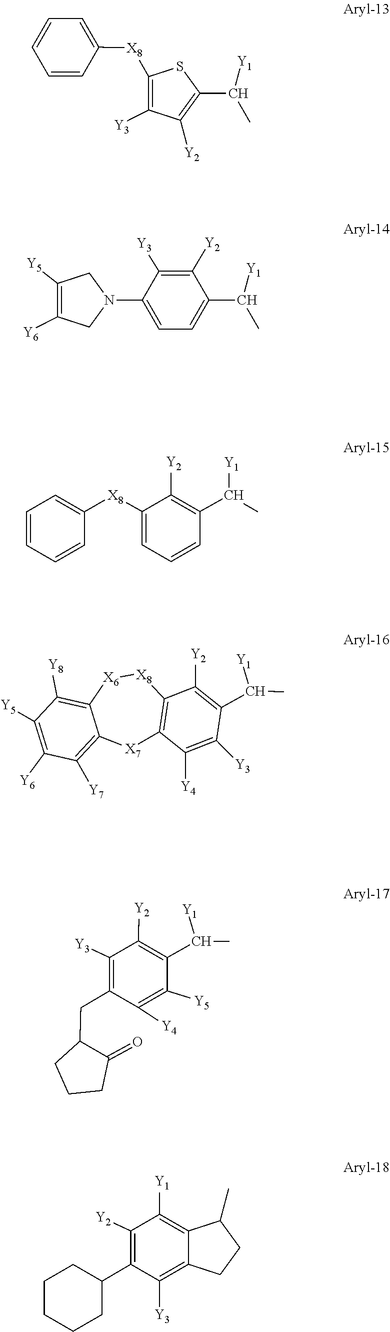 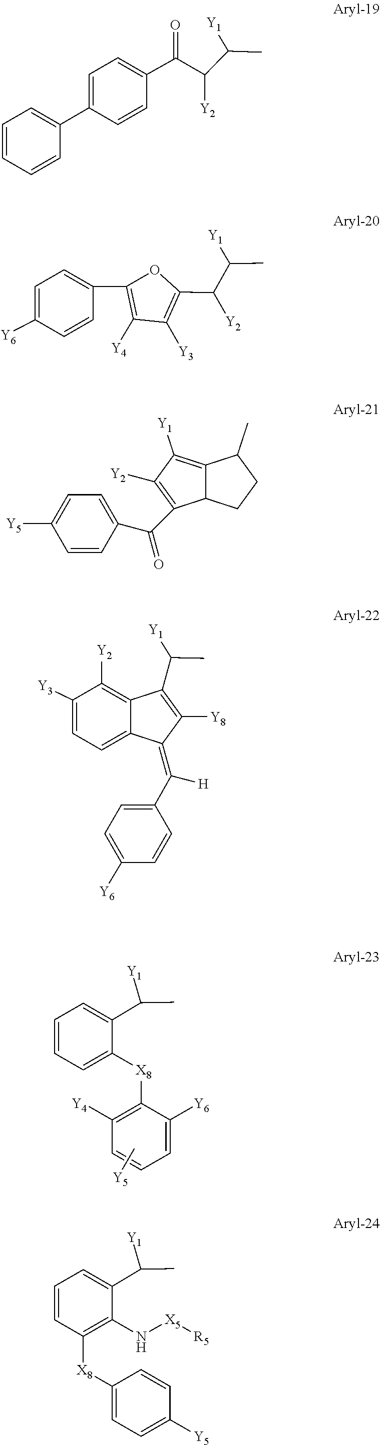 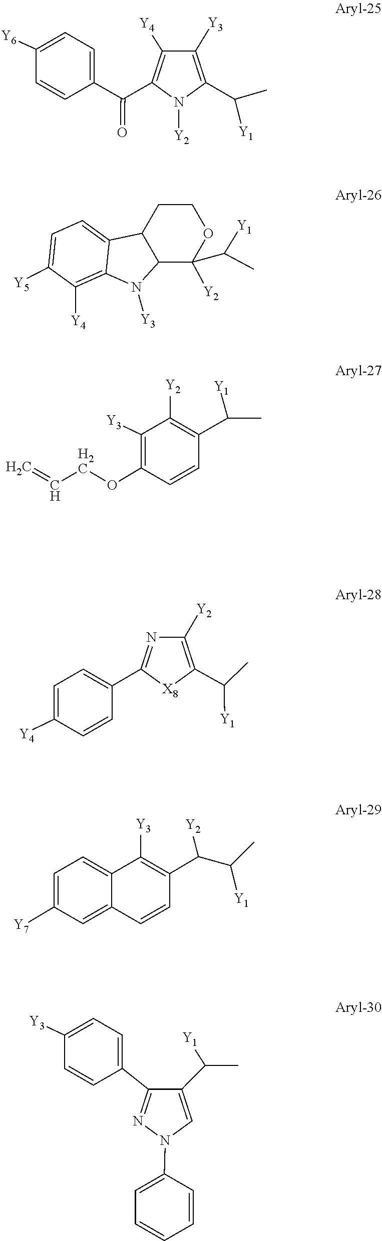 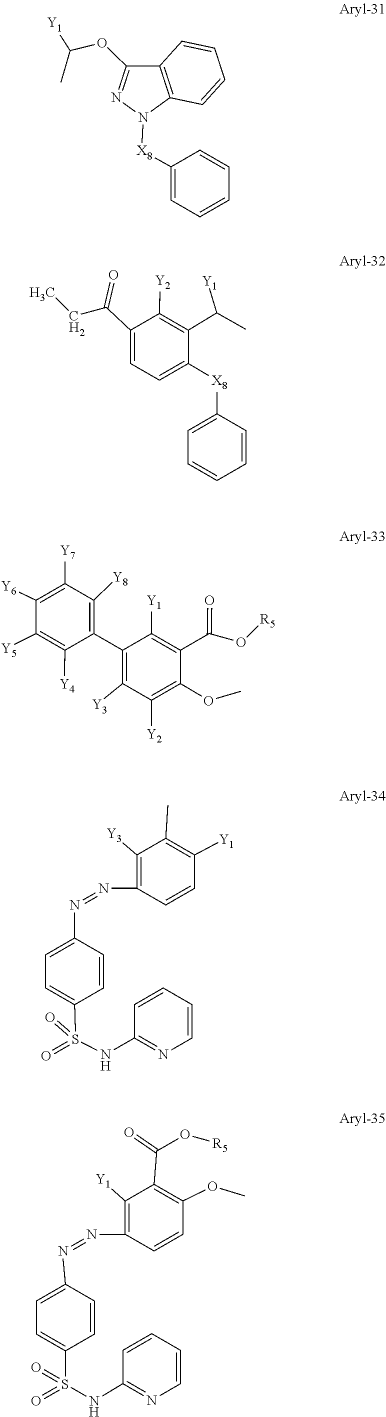 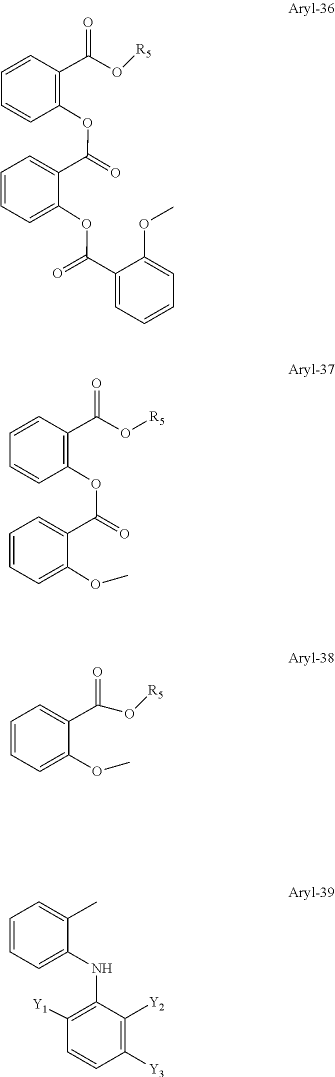 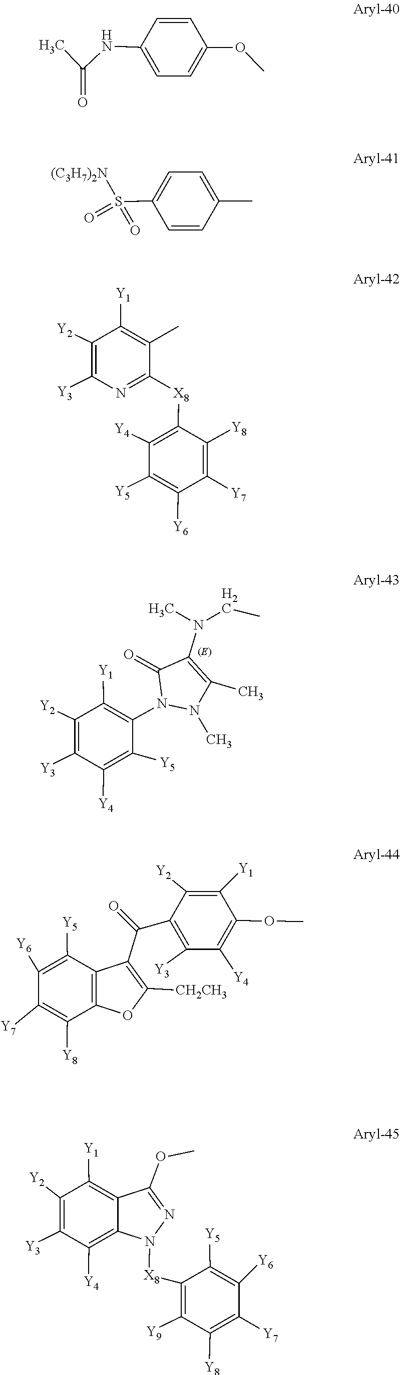 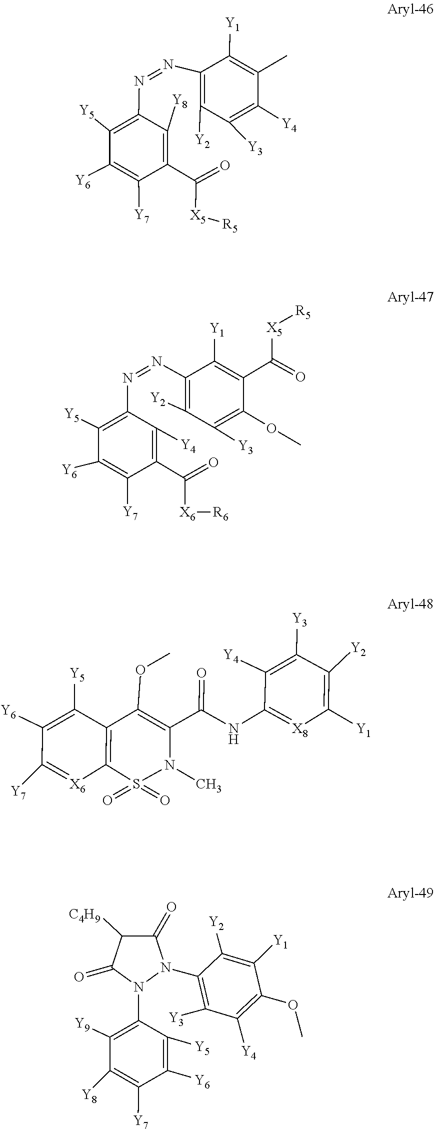 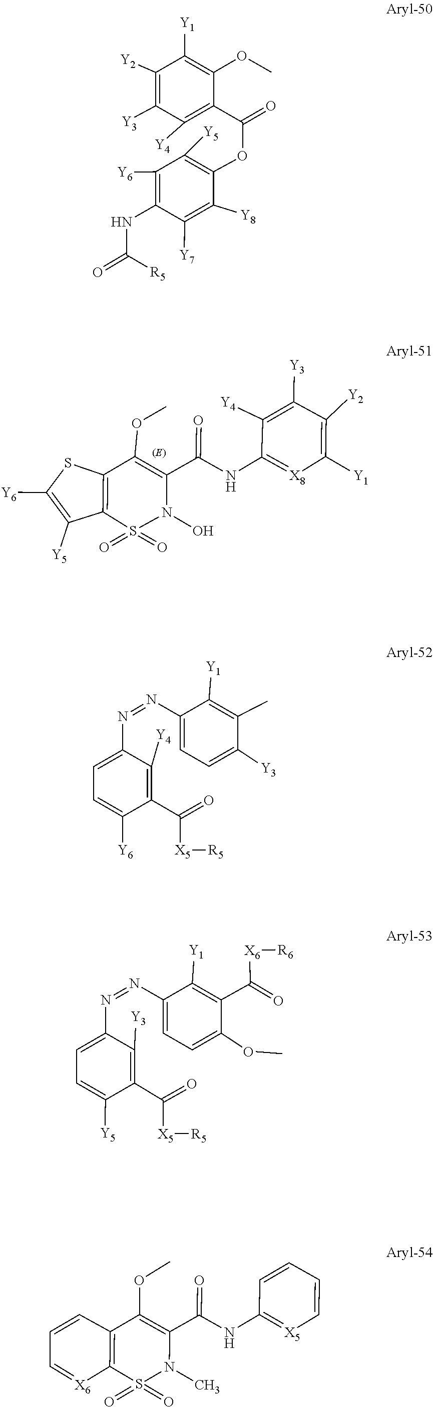 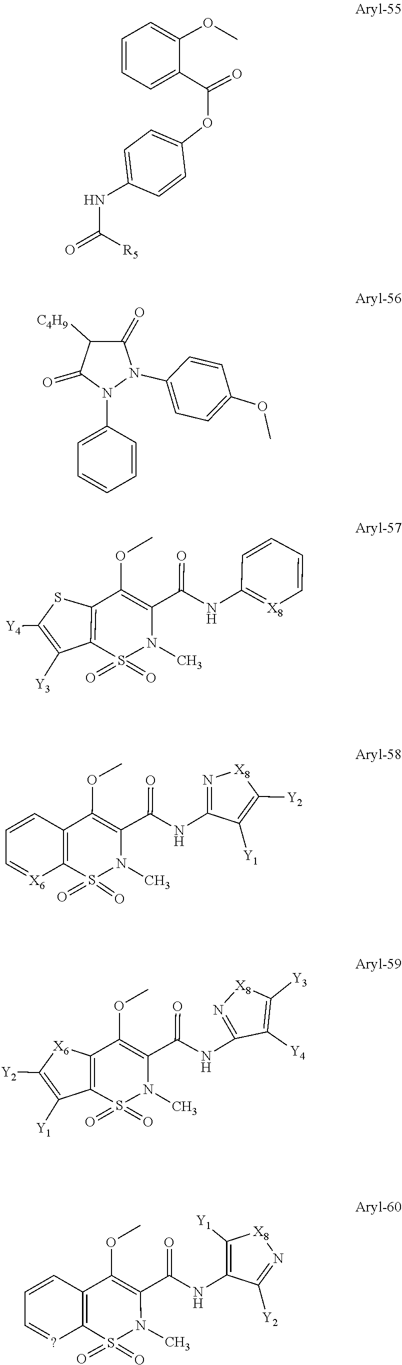 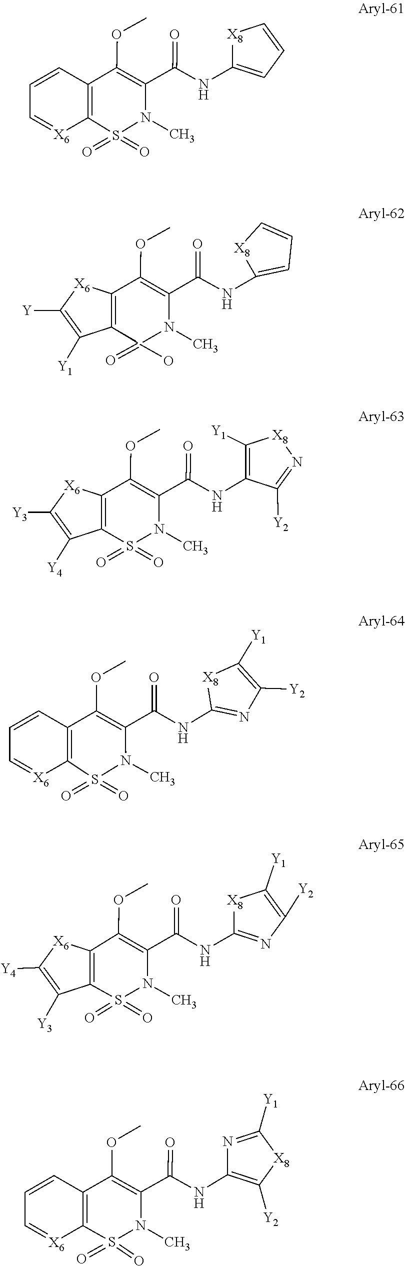 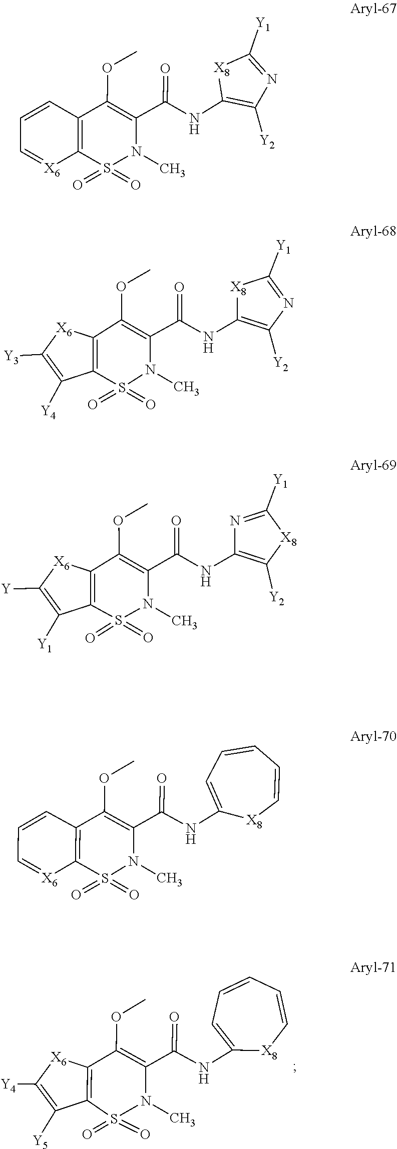
wherein:
L₁ is selected from a bond, O, S, —N(L₃)—, —N(L₃)-CH₂—O, —N(L₃)-CH₂—N(L₅)—, —O—CH₂—O—, —O—CH(L₃)-O, and —S—CH(L₃)-O—;
L₄ is selected from C—O, C═S, 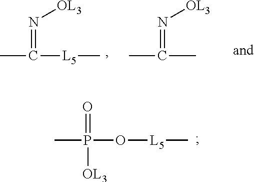
or
L₁ and L₄ together is selected from C(═O)—O, C(═O)—S, C(═O)—NH, C(═O)—O—CH(L₃)-O, C(═O)—O—CH(L₃)-S, C(═O)—O—CH(L₃)-NH, C(═O)—S—CH(L₃)-O, C(═O)—S—CH(L₃)-S, C(═O)—S—CH(L₃)-NH, P(═O) (—O-L₃)-O, O—P(═O) (—O-L₃), C(═S)—O, C(═NH)—O, C(═NH)—S, C(═N-OL₃)-NH, C(═NH-OL₃)-O, C(═NH-OL₃)-S, O—C(═O), S—C(═O), NH—C(═O), O—CH(L₃)—O—C(═O), S—CH(L₃)-O—C(═O), O—CH(L₃)-S—C(═O), S—CH(L₃)-S—C(═O), O—CH(L₃)-NH—C(═O), O—C(═S), O—C(═N-OL₃), S—C(═N-OL₃), and NH—C(═N-OL₃);
wherein each L₃ is independently H or C₁-C₆ alkyl; and
each L₅ is independently selected from a bond, CH₂COOL₆, substituted or unsubstituted C₁-C₆ alkylene, substituted or unsubstituted C₃-C₆ cycloalkylene, substituted and unsubstituted 5- to 10-membered heterocycloalkylene, substituted and unsubstituted C₆-C₁₀ arylene, and substituted and unsubstituted 5- to 10-membered heteroarylene, wherein L₆ is a bond or substituted or unsubstituted C₁-C₆ alkylene;
T is selected from Structure W-1, Structure W-2, Structure W-3, Structure W-4, Structure W-5, and Structure W-6: 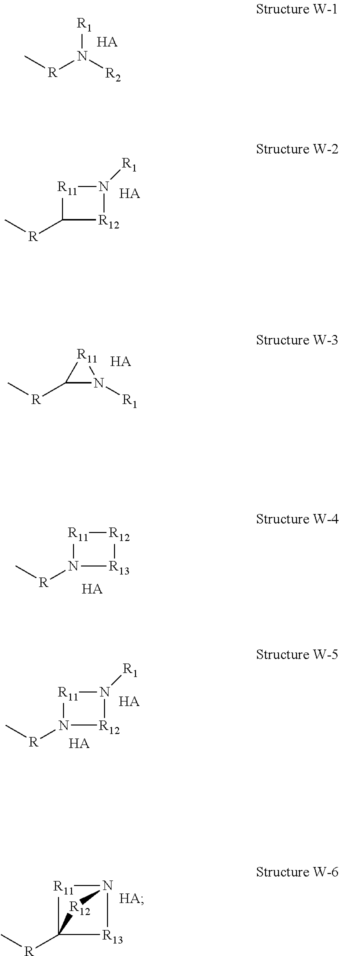
R at each occurrence is independently selected from a bond, substituted and unsubstituted alkylene, substituted and unsubstituted cycloalkylene, substituted and unsubstituted heterocyclylene;
R₁ and R₂ are independently selected from H, substituted and unsubstituted alkyl, substituted and unsubstituted cycloalkyl, and substituted and unsubstituted heterocyclyl; or alternatively R₁ and R₂ together with the nitrogen atom to which they are attached form an optionally substituted heterocyclyl, which optionally further comprises one or two additional heteroatom(s) independently selected from O, S, and N;
R₁₁, R₁₂, and R₁₃ are each independently a bond, an optionally substituted C₁-C₄ alkylene, or an optionally substituted C₂-C₄ alkyenylene, wherein the alkylene and alkenylene optionally has one CH₂ group replaced by O, S, or NR₃;
wherein any of the R₁ in Structure W-2, Structure W-3 or Structure W-5 and the adjacent R₁₁ together with the nitrogen atom to which they are attached may form an optionally substituted heterocyclic ring, which may optionally further comprise one or two additional heteroatom(s) independently selected from O, S, and N; and
wherein the R₁₁ and R₁₂ or R₁₁ and R₁₃ in Structure W-2, Structure W-4, Structure W-5, or Structure W-6 may optionally be connected by an alkylene bridge, which is optionally substituted; and
wherein HA is nothing or a pharmaceutically acceptable acid;
X₅, X₆, X₇ and X₈ are independently selected from a bond, C(═O), C(═S), OC(═O), OC(═S), CH₂, CH, S, O and NR₅;
Y, Y₁, Y₂, Y₃, Y₄, Y₅, Y₆, Y₇, and Y₈ are independently selected from H, OH, OW, OC(═O) W, L₁-L₄-L₂-W, OC(═O) CH₃, CH₃, C₂H₅, C₃H₇, C₄H₉, R₆, SO₃R₆, CH₂OR₆, CH₂OC(═O) R₆, CH₂C(═O) OR₈, OCH₃, OC₂H₅, OR₆, CH₃SO₂, R₆SO₂, CH₃SO₃, R₆SO₃, NO₂, CN, CF₃, OCF₃, CH₂ (CH₂)ₙNR₅R₆, CH₂ (CH₂)ₙOR₆, CH(C(═O) NH₂) NHR₆, CH₂C(═O) NH₂, F, Br, I, C₁, CH═CHC(═O) NHCH₂C(═O) OW, CH═CHC(═O) NHCH₂ L₁-L₄-L₂-W, NR₈C(═O)R₅, SO₂NR₅R₈, C(═O) R₅, SR₅, R₆OOCCH (NHR₇) (CH₂),C(═O) NH—, R₆OOCCH (NHR₇) (CH₂)ₙSC(═O) NH—, CF₃SCH₂C(═O) NH—, CF₃CH₂C(═O) NH—, CHF₂SCH₂C(═O) NH—, CH₂FSCH₂C(═O) NH—, NH₂C(═O) CHFS—CH₂C(═O) NH—, R₇NHCH(C(═O) OW) CH₂SCH₂C(═O) NH—, R₇NHCH (L₁-L₄-L₂-W) CH₂SCH₂C(═O) NH—, CNCH₂SCH₂C(═O) NH—, CH₃ (CH₂)ₙC(═O) NH—, R₇N═CHNR₇CH₂CH₂S—, R₇N=C(NHR₇) NHC(═O)—, R₇N=C(NHR₇) NHC(═O) CH₂, CH₃C (CI)═CHCH₂SCH₂C(═O) NH—, (CH₃)₂C (OR₆)—, CNCH₂C(═O) NH—, CNCH₂CH₂S—, R₇HN═CH(NR₇) CH₂CH₂S—, CH₂═CHCH₂SCH₂C(═O) NH—, CH₃CH(OH)—, CH₃CH(OR₈)—, CH₃CH(Y₁)—, (CH₃)₂CH—, CH₃CH₂—, CH₃ (CH₂)ₙCH═CH(CH₂)ₘC(═O) NH—, substituted and unsubstituted alkoxy, substituted and unsubstituted alkylthio, substituted and unsubstituted alkylamino, and substituted and unsubstituted alkylcarbonyl;
R₅ is independently selected from H, C(═O) NH₂, CH₂CH₂OR₆, CH₂CH₂N (CH₃)₂, CH₂CH₂N (CH₂CH₃)₂, C₁, F, Br, I, substituted and unsubstituted alkyl, substituted and unsubstituted alkenyl, substituted and unsubstituted alkynyl, substituted and unsubstituted cycloalkyl, substituted and unsubstituted heterocycloalkyl, substituted and unsubstituted alkyloxyl, substituted and unsubstituted cycloalkyloxyl, substituted and unsubstituted aryl, substituted and unsubstituted heteroaryl, substituted and unsubstituted alkylcarbonyl, substituted and unsubstituted alkylamino, —C(═O)—W, L₁-L₄-L₂-W, and W;
R₆ is independently selected from substituted and unsubstituted alkyl, substituted and unsubstituted cycloalkyl, substituted and unsubstituted heterocycloalkyl, substituted and unsubstituted alkenyl, substituted and unsubstituted alkynyl, substituted and unsubstituted alkyloxyl, substituted and unsubstituted cycloalkyloxyl, substituted and unsubstituted aryl, substituted and unsubstituted heteroaryl, —C(═O)—W,-L₁-L₄-L₂-W, and W;
R₇ is independently selected from H, F, Cl, Br, I, CH₃NHC(═O) CH₂CH(NHR₈) C(═O), R₅N=C(NHR₆) NHC(═O)—, C(═O) CH₃, C(═O) R₆, PO (OR₅) OR₆, substituted and unsubstituted alkyl, substituted and unsubstituted cycloalkyl, substituted and unsubstituted heterocycloalkyl, substituted and unsubstituted alkyloxyl, substituted and unsubstituted alkenyl, substituted and unsubstituted alkynyl, substituted and unsubstituted aryl, substituted and unsubstituted heteroaryl, substituted and unsubstituted alkylcarbonyl, substituted and unsubstituted alkylamino, L₁-L₄-L₂-W, and C—(═O)—W;
R₈ is independently selected from H, F, Cl, Br, I, CH₃, C₂H₅, CF₃, CH₂CH₂F, CH₂CH₂Cl, CH₂CH₂Br, CH₂CH₂I, CH₂CHF₂, CH₂CF₃, CH₂F, CH₂Cl, CH₂Br, CH₂I, CH₂NR₆R₇, CH(NHR₇) CH₂C(═O) NH₂, C₃H₇, C₄H₉, C₆H₁₁, R₆, C(═O) R₆, C(═O) NH₂, CH₂C(═O) NH₂, CH₂OC(═O) NH₂, PO (OR₅) OR₆, C(CH₃)₂C(═O) OR₆, CH(CH₃) C(═O) OR₆, CH₂C(═O) OR₆, C(═O)—W, and L₁-L₄-L₂-W;
L₂ is independently selected from a bond, O, S, —N(L₃)—, —N(L₃)-CH₂-0, —N(L₃)-CH₂—N (L₃)—, —O—CH₂—O—, —O—CH(L₃)-O, —S—CH(L₃)—O—, —O-L₅-, —S-L₅-, and —N(L₃)-L₅-; and
W is selected from H, substituted and unsubstituted alkyl, substituted and unsubstituted cycloalkyl, substituted and unsubstituted heterocycloalkyl, substituted and unsubstituted alkyloxy, substituted and unsubstituted alkenyl, substituted and unsubstituted alkynyl, substituted and unsubstituted aryl, substituted and unsubstituted heteroaryl, Structure W-1, Structure W-2, Structure W-3, Structure W-4, Structure W-5, and Structure W-6;
provided that the structure of HPP as defined forms a stable compound without any covalent bond principles being violated.

13. The method according to claim 1, wherein the HPP is selected from:
(1) (pyrrolidin-2-yl)methyl 2-(2-(2,6-dichlorophenylamino)phenyl) acetate;
(2) 2-(diethylamino)ethyl 2-(4-isobutylphenyl) propanoate;
(3) 2-(N,N-diethylamino)ethyl 2-[1-[[(1R)-1-[3-[2-(7-chloroquinolin-2-yl) ethenyl]phenyl]-3-[2-(2-hydroxypropan-2-yl)phenyl]propyl]sulfanylmethyl]cyclopropyl]acetate;
(4) 2-(diethylamino)ethyl acetoxybenzoate;
(5) (RS)—N-[1-(1-benzothien-2-yl)ethyl]-N-(2-N,N-diethylaminoacetyloxy) urea;
(6) (pyrrolidin-2-yl)methyl 2-cyclopentyl-2-[4-(quinolin-2-ylmethoxy)phenyl]acetate;
(7) (pyrrolidin-2-yl)methyl 3-[[3-[(E)-2-(7-chloroquinolin-2-yl) ethenyl]phenyl]-[3-(dimethylamino)-3-oxopropyl]sulfanylmethyl]sulfanylpropanoate;
(8) (Z)-2-(N,N-diethylaminoethyl) ethyl 2-(5-fluoro-2-methyl-1-(4-methylsulfinyl)benzylidene)-1H-inden-1-yl) acetate;
(9) 2-(dimethylamino)ethyl 2-(3-phenoxyphenyl) propionate,
(10)S-(2-(dimethylamino)ethyl 2-(3-phenoxyphenyl) propanethioate;
(11) 2-(dipropylamino)ethyl 4-acetoxy-2′,4′-difluoro-[1,1′-biphenyl]-3-carboxylate hydrochloride [2-(dipropylamino)ethyl 5-(2,4-difluorophenyl) acetylsalicylate;
(12) (pyrrolidin-2-yl)methyl 3-[[1-(4-chlorobenzyl)-4-methyl-6-(5-phenylpyridin-2-yl) methoxy]-4,5-dihydro-1H-thiopyrano[2,3,4-c,d]indol-2-yl]-2,2-dimethylpropanoate;
(13) (pyrrolidin-2-yl)methyl 5-benzoyl-2,3-dihydro-1H-pyrrolizine-1-carboxylate;
(14) (pyrrolidin-2-yl)methyl 1,8-diethyl-1,3,4,9-tetrahydropyrano[3,4-b]indole-1-acetate;
(15) (pyrrolidin-2-yl)methyl 2-(2-fluorobiphenyl-4-yl) propanoate;
(16) (pyrrolidin-2-yl)methyl 2-(6-methoxy-2-naphthyl) propanoate;
(17) (pyrrolidin-2-yl)methyl 2-[(2,3-dimethylphenyl)amino]benzoate;
(18) (pyrrolidin-2-yl)methyl 6-chloro-α-methylcarbazole-2-acetate;
(19) (pyrrolidin-2-yl)methyl 4-[4-[3-(4-acetyl-3-hydroxy-2-propylphenoxy) propylsulfonyl]phenyl]-4-oxo-butanoate;
(20) (pyrrolidin-2-yl)methyl (Z)-5-fluoro-2-methyl-1-[(4-methylsulfinyl)phenylmethylene]-1H-indene-3-acetate;
(21) (pyrrolidin-2-yl)methyl 1-methyl-5-(4-methylbenzoyl)-1H-pyrrole-2-acetate;
(22) (pyrrolidin-2-yl)methyl 1-(4-chlorobenzoyl)-5-methoxy-2-methylindole 3-acetate;
(23) (pyrrolidin-2-yl)methyl 5-(4-chlorobenzoyl)-1,4-dimethyl-1H-pyrrole-2-acetate;
(24) (pyrrolidin-2-yl)methyl 3-chloro-4-(2-propenyloxy)benzeneacetate;
(25) (pyrrolidin-2-yl)methyl 2-(4-isobutylphenyl) propanoate;
(26) (pyrrolidin-2-yl)methyl 2-acetoxybenzoate;
(27) (pyrrolidin-2-yl)methyl 5-(2,4-difluorophenyl) salicylate;
(28) (pyrrolidin-2-yl)methyl 5-(2,4-difluorophenyl) acetylsalicylate;
(29) (pyrrolidin-2-yl)methyl 2-(3-benzoylphenyl) propionate;
(30) (pyrrolidin-2-yl)methyl 2-(3-phenoxyphenyl) propionate hydrochloride;
(31) (pyrrolidin-2-yl)methyl (Z)-5-fluoro-2-methyl-1-[(4-methylsulfinyl)phenylmethylene]-1H-indene-3-acetate;
(32) (pyrrolidin-2-yl)methyl 5-(4-chlorobenzoyl)-1,4-dimethyl-1H-pyrrole-2-acetate;
(33) (pyrrolidin-2-yl)methyl α-methyl-4-(2-thienylcarbonyl)benzeneacetate;
(34) (pyrrolidin-2-yl)methyl 6-chloro-α-methyl-9H-carbazole-2-acetate;
(35) (pyrrolidin-2-yl)methyl 2-(4-chlorophenyl)-α-methyl-5-benzoxazoleacetate;
(36) (pyrrolidin-2-yl)methyl 2-[3-chloro-4-(3-pyrrolin-1-yl)phenyl]propanoate;
(37) (pyrrolidin-2-yl)methyl α-methyl-(4-chlorobenzoyl)-5-methoxy-2-methylindole-3-acetate;
(38) (pyrrolidin-2-yl)methyl 2-(5H-[1]-Benzopyrano[2,3-b]pyridin-7-yl) propionate;
(39) (pyrrolidin-2-yl)methyl α-methyl-4-[(2-methyl-2-propen-1-yl)amino]benzeneacetate;
(40) (pyrrolidin-2-yl)methyl 5-benzoyl-α-methyl-2-thiopheneacetate;
(41) (pyrrolidin-2-yl)methyl 10,11-Dihydro-α-methyl-10-oxodibenzo[b,f]thiepin-2-acetate;
(42) (pyrrolidin-2-yl)methyl 2-(8-methyl-5-oxo-6H-benzo[b][1]benzoxepin-3-yl)propanote;
(43) (pyrrolidin-2-yl)methyl 2-[4-[(2-oxocyclopentyl)methyl]phenyl]propanote;
(44) (pyrrolidin-2-yl)methyl 4-(1,3-dihydro-1-oxo-2H-isoindol-2-yl)-α-methylbenzeneacetate;
(45) (pyrrolidin-2-yl)methyl 2-chloro-2-(3-chloro-4-cyclohexylphenyl) acetate;
(46) (pyrrolidin-2-yl)methyl 4,5-diphenyl-2-oxazolepropanoate;
(47) (pyrrolidin-2-yl)methyl 3-(4-biphenylcarbonyl) propanoate;
(48) (pyrrolidin-2-yl)methyl 3-[5-(4-chlorophenyl) furan-2-yl]-3-hydroxypropanoate;
(49) (pyrrolidin-2-yl)methyl 6-chloro-5-cyclohexyl-1-indancarboxylate hydrochloride;
(50) (pyrrolidin-2-yl)methyl 1-(4-chlorobenzoyl)-5-methoxy-2-methylindole 3-acetate;
(51) (pyrrolidin-2-yl)methyl 1-methyl-5-(4-methylbenzoyl)-1H-pyrrole-2-acetate;
(52) (pyrrolidin-2-yl)methyl 5-(4-chlorobenzoyl)-1,4-dimethyl-1H-pyrrole-2-acetate;
(53) (pyrrolidin-2-yl)methyl 3-chloro-4-(2-propenyloxy)benzeneacetate;
(54) (pyrrolidin-2-yl)methyl 3-2-(2,4-dichlorophenoxy)benzeneacetate;
(55) (pyrrolidin-2-yl)methyl 4-acetamidophenyl salicylate;
(56) (pyrrolidin-2-yl)methyl 2-[(2,3-dimethylphenyl)amino]benzoate;
(57) (pyrrolidin-2-yl)methyl 2-[(2,3-dimethylphenyl)amino]benzoate;
(58) (pyrrolidin-2-yl)methyl 2-[3-(trifluoromethyl)phenyl]amino]-3-pyridinecarboxylate;
(59) (pyrrolidin-2-yl)methyl 2-[(2,6-dichloro-3-methylphenyl)amino]benzoate;
(60) (pyrrolidin-2-yl)methyl 2-[[2-methyl-3-(trifluoromethyl)phenyl]amino]-3-pyridinecarboxylate;
and stereoisomers and pharmaceutically acceptable salts thereof.

14. The method according to claim 6, wherein HA is selected from a group consisting of hydrochloride hydrobromide, hydroiodide, nitric acid, sulfic acid, bisulfic acid, phosphoric acid, phosphorous acid, phosphonic acid, isonicotinic acid, acetic acid, lactic acid, salicylic acid, citric acid, tartaric acid, pantothenic acid, bitartaric acid, ascorbic acid, succinic acid, maleic acid, gentisinic acid, fumaric acid, gluconic acid, glucaronic acid, saccharic acid, formic acid, benzoic acid, glutamic acid, methanesulfonic acid, ethanesulfonic acid, benzensulfonic acid, p-toluenesulfonic acid, and pamoic acid.

15. The method according to claim 1, wherein said administering the HPP or the stereoisomer or pharmaceutically acceptable salt thereof comprising administering a pharmaceutical composition comprising: the HPP or the stereoisomer or pharmaceutically acceptable salt thereof, and a pharmaceutically acceptable carrier.

16. The method according to claim 15, wherein the pharmaceutically acceptable carrier is selected from alcohol, acetone, ester, cellulose, mannitol, croscarmellose sodium, vegetable oil, hydroxypropyl methylcellulose, water, and aqueous solution.

17. The method according to claim 1 wherein HA is hydrochloride.

18. The method of claim 1, wherein:
(1) the viral infection is caused by an RNA virus, DNA virus, an influenza virus, or a coronavirus;
(2) the bacterial infection is caused by a gram-positive, gram-negative, or pathogenic bacteria;
(3) the protozoal infection is caused a pathogenic protozoa that cause diseases in humans or animals;
(4) the sign or symptom is selected from fatigue, loss of appetite, weight loss, fevers, night sweats, chills, aches, inflammation, cough, shortness of breath, pains, runny nose, and combinations thereof; and
(5) the complication is selected from pneumonia, acute respiratory distress syndrome, chronic obstructive pulmonary disease, blood clotting (coagulation), meningitis, encephalitis, cardiovascular disease, stroke, heart attack, sepsis, and inflammation of the brain, lung, kidney, liver, pancreas, GI system, blood vessels, and other tissues, and combinations thereof.

19. The method according to claim 18, wherein:
(1) the influenza virus is:
(a) a Human influenza virus selected from H1N1, H1N2 H1N7 H2N2, H2N3, H3N1, H3N2, H3N8, H5N1, H5N2, H5N3, H5N6, H5N8, H5N9, H6N1, H6N2, H6N5, H7N1, H7N2, H7N3, H7N4, H7N9, H7N7, H8N4, H9N2, H10N7, H10N8, H10N3, H11N2, H11N9, H12N5, H13N6, H17N10, and H18N11;
(b) an avian influenza A virus selected from H1N1, H1N8, H2N9, H3N8, H3N2, H4N6, H4N3, H5N4, H5N8, H5N9, N5N1, H6N2, H6N1, H6N5, H6N8, H7N1, H8N4 H9N2, H9N2, H9N6, H9N7, H10N8, H11N6, H11N9, H12N5, H13N6, H14N4, and H15N9;
(c) a swine influenza A virus selected from H1N1, H1N2, H2N1, H3N2, and H2N3;
(d) an equine influenza virus selected from H3N8 and H7N7;
(e) a canine influenza virus selected from H3N2, H3N8, and H5N1; or
(f) a cat influenza virus selected from Feline herpes virus, Feline Calicivirus, Bordetella, bronchiseptica, and Chlamydophila felis;
(2) the coronavirus is selected from severe acute respiratory syndrome coronavirus (SARS—Co—V), SARS-COV-1, SARS-COV-2 (Covid-19), 229E, NL63, OC₄₃, HKU1, MERS-COV, and the original SARS-COV;
(3) the pathogenic bacteria is selected from tuberculosis, streptococcus, syphilis, staphylococcus, aspergillus, tetanus, Vibrio cholera, salmonella, Clostridium botulinum, and E, Coli; and
(4) the pathogenic protozoa is selected from Entamoeba histolytica (Amoebozoa), Acanthamoeba (Amoebozoa), Giardia lamblia (Metamonada), Trichomonas vaginalis (Metamonada), Dientamoeba fragilis (Metamonada), Trypanosoma brucei (Kinetoplastida), Trypanosoma cruzi (Kinetoplastida), Leishmania spp. (Kinetoplastida), Balantidium coli (Ciliate), Plasmodium spp. (Apicomplexa), Toxoplasma gondii (Apicomplexa), Babesia spp. (Apicomplexa), Cryptosporidium spp. (Apicomplexa), and Cyclospora cayetanensis (Apicomplexa).

20. The method of claim 1, selected from:
(1) the method of treating signs, symptoms, and complications of H1N1 virus and other flu viruses infections in the subject, comprising administering 2-(diethylamino)ethyl 2-(4-isobutylphenyl) propanoate hydrochloride, 2-(diethylamino)ethyl 2-acetoxy benzoate hydrochloride, (pyrrolidin-2-yl)methyl 2-(2-(2,6-dichlorophenylamino)phenyl) acetate hydrochloride, (pyrrolidin-2-yl)methyl 2-(6-methoxy-2-naphthyl) propanoate hydrochloride, (pyrrolidin-2-yl)methyl 2-(2-fluorobiphenyl-4-yl) propanoate hydrochloride, (Z)-2-(N,N-diethylaminoethyl) ethyl 2-(5-fluoro-2-methyl-1-(4-methylsulfinyl)benzylidene)-1H-inden-1-yl) acetate ACOH, (pyrrolidin-2-yl)methyl 2-[(2,3-dimethylphenyl)amino]benzoate hydrochloride, (pyrrolidin-2-yl)methyl α-methyl-4-(2-thienylcarbonyl)benzeneacetate hydrochloride, (pyrrolidin-2-yl)methyl 5-(2,4-difluorophenyl) salicylate hydrochloride, or (pyrrolidin-2-yl)methyl 5-(2,4-difluorophenyl) acetylsalicylate hydrochloride to the subject;
(2) the method of treating signs, symptoms, and complications of COVID-19 and other coronaviruses infections in the subject, comprising administering 2-(diethylamino)ethyl 2-(4-isobutylphenyl) propanoate hydrochloride, 2-(diethylamino)ethyl 2-acetoxybenzoate hydrochloride, (pyrrolidin-2-yl)methyl 2-(2-(2,6-dichlorophenylamino)phenyl) acetate hydrochloride, (pyrrolidin-2-yl)methyl 2-(6-methoxy-2-naphthyl) propanoate hydrochloride, (pyrrolidin-2-yl)methyl 5-benzoyl-2,3-dihydro-1H-pyrrolizine-1-carboxylate hydrochloride, (pyrrolidin-2-yl)methyl 1,8-diethyl-1,3,4,9-tetrahydropyrano[3,4-b]indole-1-acetate hydrochloride, (pyrrolidin-2-yl)methyl 2-[3-chloro-4-(3-pyrrolin-1-yl)phenyl]propanoate hydrochloride, (pyrrolidin-2-yl)methyl 2-(5H-[1]-benzopyrano[2,3-b]pyridin-7-yl) propionate hydrochloride, pyrrolidin-2-yl)methyl 4,5-diphenyl-2-oxazolepropanoate hydrochloride, or (pyrrolidin-2-yl)methyl 2-(3-benzoylphenyl) propionate hydrochloride to the subject;
(3) the method of treating viral, bacterial, protozoal, and/or fungal infections-induced sepsis in the subject, comprising administering 2-(diethylamino)ethyl 2-acetoxybenzoate hydrochloride, (pyrrolidin-2-yl)methyl 2-(2-(2,6-dichlorophenylamino)phenyl) acetate hydrochloride, (pyrrolidin-2-yl)methyl 2-(3-phenoxyphenyl) propionate hydrochloride, (pyrrolidin-2-yl)methyl salicylsalicylate hydrochloride, (pyrrolidin-2-yl)methyl 2-[[2-[(2,6-dichlorophenyl)amino]phenyl]acetoxy]acetate hydrochloride, (pyrrolidin-2-yl)methyl 1-methyl-5-(4-methylbenzoyl)-1H-pyrrole-2-acetate hydrochloride, (pyrrolidin-2-yl)methyl 5-(4-chlorobenzoyl)-1,4-dimethyl-1H-pyrrole-2-acetate hydrochloride, or (pyrrolidin-2-yl)methyl dichlorophenoxy)benzeneacetate hydrochloride to the subject;
(4) the method of treating drug-resistant viral, bacterial, protozoal, and/or fungal infections-induced sepsis in the subject, comprising administering 2-(diethylamino)ethyl 2-acetoxybenzoate hydrochloride, (pyrrolidin-2-yl)methyl 2-(2-(2,6-dichlorophenylamino)phenyl) acetate hydrochloride, (pyrrolidin-2-yl)methyl 2-(3-phenoxyphenyl) propionate hydrochloride, 2-(diethylamino)ethyl 2-(4-isobutylphenyl) propanoate hydrochloride, (pyrrolidin-2-yl)methyl 2-[2-[(2,6-dichlorophenyl)amino]phenyl]acetoxy]acetate hydrochloride, (pyrrolidin-2-yl)methyl 1-methyl-5-(4-methylbenzoyl)-1H-pyrrole-2-acetate hydrochloride, (pyrrolidin-2-yl)methyl 5-(4-chlorobenzoyl)-1,4-dimethyl-1H-pyrrole-2-acetate hydrochloride, or (pyrrolidin-2-yl)methyl dichlorophenoxy)benzeneacetate hydrochloride to the subject; and
(5) the method of treating signs, symptoms and complications of methicillin-resistant Staphylococcus aureus (MRSA) infection in the subject, comprising administering 2-(diethylamino)ethyl 2-acetoxybenzoate hydrochloride, (pyrrolidin-2-yl)methyl 2-(2-(2,6-dichlorophenylamino)phenyl) acetate hydrochloride, (pyrrolidin-2-yl)methyl 2-(6-methoxy-2-naphthyl) propanoate hydrochloride, 2-(diethylamino)ethyl 2-(4-isobutylphenyl) propanoate hydrochloride, (pyrrolidin-2-yl)methyl 5-benzoyl-2,3-dihydro-1H-pyrrolizine-1-carboxylate hydrochloride, or (pyrrolidin-2-yl)methyl 1,8-diethyl-1,3,4,9-tetrahydropyrano[3,4-b]indole-1-acetate hydrochloride to the subject.
